(19) United States
(12) Patent Application Publication
(10) Pub. No.: US 2026/0234735 A1
(43) Pub. Date: Aug. 13, 2026

(54) Probes for Distinction of Sterile and Bacterial Infection

(71) Applicant: Helmholtz Zentrum München Deutsches Forschungszentrum für Gesundheit und Umwelt (GmbH), Neuherberg (DE)

(72) Inventors: Oliver Plettenburg, Neuherberg (SE); Fabian Brunswig, Neuherberg (SE); Tobias Ross, Neuherberg (SE); Frank M. Bengel, Neuherberg (SE); Silay Al-Bazaz, Neuherberg (SE)

(21) Appl. No.: 19/156,778

(22) PCT Filed: Feb. 21, 2024
(86) PCT No.: PCT/EP2024/054451

§ 371 Date: Aug. 14, 2025

(30) Foreign Application Priority Data
Feb. 21, 2023 (EP) 23157867.5

(51) Int. Cl. C12Q 1/689

(52) U.S. Cl. CPC C12Q 1/689; C12Q 2600/156

(57) ABSTRACT

The present invention relates to a DNA-probe comprising
a) a DNA-binder (DNAB);
b) a tracer (T)
c) optionally a linker (L) connecting the DNA-binder (DNAB) and the tracer (T).

### TECHNICAL FIELD OF THE INVENTION

The present invention relates to a DNA-probe comprising
a) a DNA-binder (DNAB);
b) a tracer (T)
c) optionally a linker (L) connecting the DNA-binder (DNAB) and the tracer (T).

### BACKGROUND ART

In 2019, the world health organization (WHO) declared antimicrobial resistances as one of the top 10 threats for public health facing humanity. Mis- and overuse of antibiotic accelerate the development of drug resistant pathogens. Illnesses with uncertain symptoms are often treated with antibiotics, in expectance of bacteria being the cause for the infection. But not only the overuse of antibiotics in inappropriate cases is a problem.

Joint replacement surgery, one of the most successful procedures in orthopedics, remains the ultimate option to relieve uncontrolled pain and reestablish joint function in end-stage hip and knee arthritis (Learmonth et al., 2007). A regression analysis with age, gender, race and/or ethnicity, census region, and year as covariates, performed using data from the US National Center for Health Statistics, indicate that the number of hip and knee arthroplasties is estimated to grow 174% to 572,000 procedures and 673% to 3.48 million, respectively, by 2030 (Kurtz et al., 2007). The same study projects IDF—Strictly confidential 4 that about 7 and 15% of knee and hip arthroplasties, respectively, are still expected to fail, causing the need for a revision surgery (Kurtz et al., 2007).

The revision surgeries, in addition to the significant healthcare costs, are associated with a high risk of infection and poor clinical outcomes (Vanhegan et al., 2012; Weber et al., 2018). On the other hand, a small percentage of patients undergoing hip or knee replacement (roughly about 1 in 100) may develop an infection after the operation. Joint replacement infections may occur in the wound or deep around the artificial implants. The unambiguous non-invasive distinction of a bacterial infection over a sterile inflammation can be troublesome, as there is a significant overlap in symptoms and also in the findings of standard imaging techniques (CT, MRI and PET). For example, FDG PET studies do not lead to conclusive results. In both cases, rapid initiation of tailored therapy is warranted, either dosing antibiotic or anti-inflammatory and/or immunosuppressive therapy. A misdiagnosis can have severe consequences, e.g. losing the implant. Classical methods for the detection of bacterial infections exist, but these often rely on the examination and cultivation of tissue samples taken from the suspected site. These methods are effective but are laborious, complex and time-consuming due to their invasive nature. In the case of acute inflammation, the result may be available too late to guide clinical decision on therapy.

Hence, the development of a non-invasive diagnosis tool is an urgent medical need.

### SUMMARY OF THE INVENTION

The invention is directed to a DNA-probe according to formula (I) or (II) comprising
a) a DNA-binder (DNAB);
b) a tracer (T)
c) optionally a linker (L) connecting the DNA-binder (DNAB) and the tracer (T)

(DNAB)-(L)-(T) or (I)

(DNAB)-(T). (II)

The invention is further directed to the DNA-probe for use in detection of extracellular DNA of bacteria.

The DNA-probe of the present invention has the capability to solely bind bacterial DNA, more specifically extracellular DNA of bacteria (eDNA). Bacteria can life planktonic, but favour a protected colony called biofilm. A biofilm is a community of microorganisms embedded in a slimy matrix consisting of excreted extracellular polymeric substances (EPS) forming a hydrogel. These substances are carbohydrates, proteins, lipids and DNA. The particularity of this DNA is, that it is located extracellular (eDNA). In case of a bacterial infection, eDNA will be enriched near the infection site and this way, the eDNA works as a protective layer, limiting flux for bacterial existence. Thus, the DNA-probe has the ability to bind (e)DNA, is traceable through the body and has a non-cell permeable character.

A suitable tracing method is based on nuclear imaging techniques such as positron emission tomography (PET), which, when combined with a morphological imaging. procedure such as computed tomography (CT) or magnetic resonance imaging (MRI), can provide a three-dimensional image of the processes in the whole body. The probe is non-cell permeable for two reasons: A cell permeable DNA interacting agent would interact also with mammalian DNA and this is a) potentially mutagenic for the patient and b) would lead to false positive results. To enable the interaction of the probe with the eDNA, a DNA-binding moiety is one of the three building blocks of the probe. Since the tracing of the probe is preferably based on radioactivity, the tracer preferably offers a conjugation site for a radioactive atom. Radioactive metal-isotopes are often used in radioactive imaging techniques, which can be easily chelated with an appropriate chelator. The tracers of the DNA-probe of the present invention do not only chelate the tracing atom, the nitrogen atoms and the carboxylic acids add a high polarity to the molecule, which is favourable in order to obtain a non cell-permeable probe. The linking system is not only the connecting unit between the DNA-binding moiety and tracer, but also offers the possibility to compliment the high polarity of the chelating moiety. In one embodiment, a PEG-chain can be a utilized as a linker, but also branched linking system, derivated from TRIS may be employed. An embodiment with TRIS based linking system offers more positions for functionalisation and by addition of e.g. charged residues or additional chelating moieties, the cell impermeability can be adjusted.

### BRIEF DESCRIPTION OF THE FIGURES

FIG. 1: DNA binding assay for probe 50

FIG. 2: DNA binding assay for probe 55

FIG. 3: Radiolabelling; HPLC (method as described): t_{R}=10.08 min, recorded HPLC chromatograms of probe 50 and the labeled probe 50*. a) UV trace of unlabelled probe 50; b) radio trace of labelled probe 50*.

FIG. 4 Residual radioactivity of the incubation experiments in various bacterial strains, normalized to the detected amount of DNA.

### DETAILED DESCRIPTION OF THE INVENTION

The solution of the present invention is described in the following, exemplified in the appended examples, illustrated in the Figures and reflected in the claims.

### Definitions

The term “alkyl” refers to a monoradical of a saturated straight or branched hydrocarbon. Preferably, the alkyl group comprises from 1 to 6 carbon atoms, i.e., 1, 2, 3, 4, 5, or 6, carbon atoms, more preferably 1 to 4 carbon atoms. Exemplary alkyl groups include methyl, ethyl, propyl, iso-propyl, butyl, iso-butyl, tert-butyl, n-pentyl, iso-pentyl, sec-pentyl, neo-pentyl, 1,2-dimethyl-propyl, iso-amyl, n-hexyl, iso-hexyl, sec-hexyl, and the like.

The term “halogen” or “halo” means fluoro, chloro, bromo, or iodo.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

A better understanding of the present invention and of its advantages will be had from the following examples, offered for illustrative purposes only. The examples are not intended to limit the scope of the present invention in any way.

The invention is directed to a DNA-probe according to formula (I) or (II) comprising
a) a DNA-binder (DNAB);
b) a tracer (T)
c) optionally a linker (L) connecting the DNA-binder (DNAB) and the tracer (T)

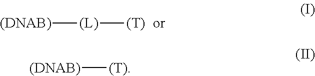

The DNA-probe is designed to bind to DNA with the DNA-binder (DNAB) and to be detected by the tracer (T) part of the DNA-probe in order to detect for example extracellular DNA.

DNA-binder (DNAB) are generally known to the person skilled in the art. The DNA-binder disclosed in the references Gozieba et al., Current Cancer Drug Targets, 2020, 20, 19-32 and Sharma et al., Current Pharmaceutical Design, 2021, 27, 15-42 are included in the invention by reference.

Within the present invention,
a) a hydrogen atom at a heteroatom in the DNA-binder (DNAB) is replaced by Y, wherein Y is connected to the linker (L) or to the tracer (T) or
b) a hydrogen atom connected to a carbon atom is replaced by Z, wherein Z is connected to the linker (L) or to the tracer (T);
and wherein
Y is a bond;
Z is selected from a group consisting of —O—,—S—, —NH—, —N((C₁-C₅)alkyl)- or a bond.

Preferably, the DNA-binder (DNAB) is selected from the group consisting of

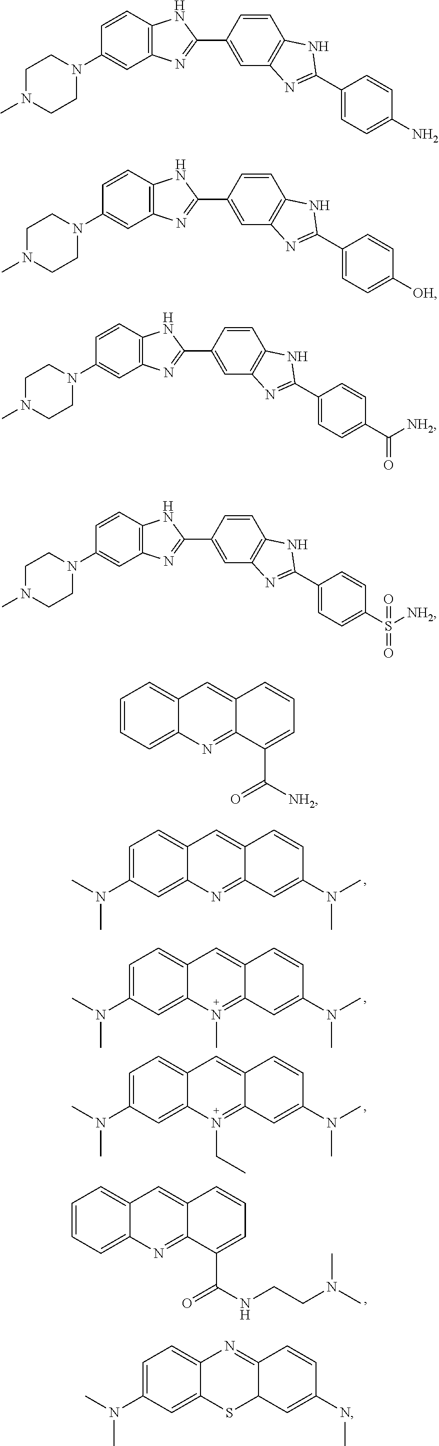

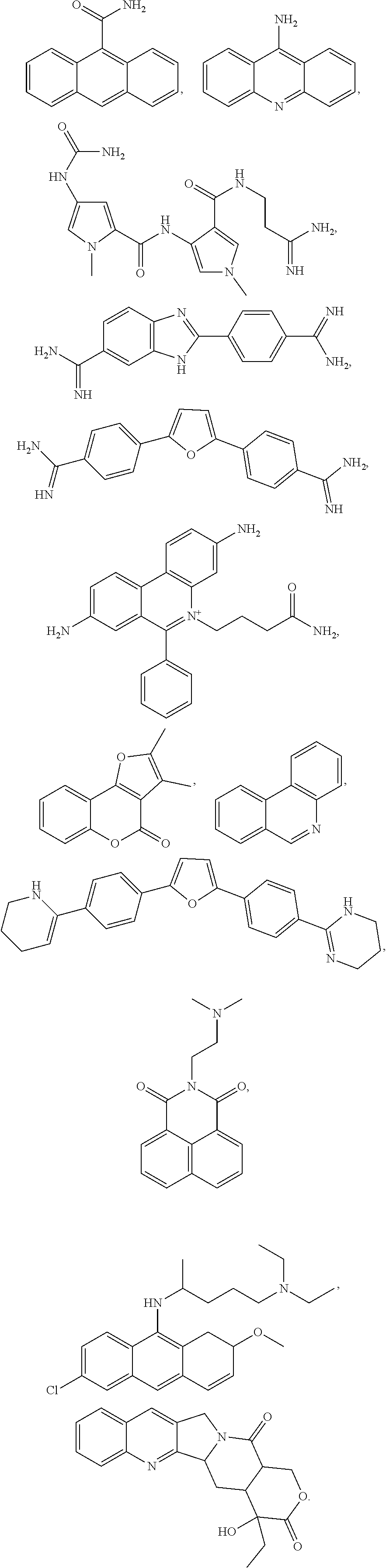

The tracer (T) allows detection of the DNA-Probe, preferably ex vivo and in vivo. The tracer (T) may be any suitable chemical group which is detectable. Preferably, the tracer (T) is a group which may form a chelate complex with a suitable detectable metal in form of an ion, such as a radioactive metal ion. Preferably, the radioactive metal ion is selected from the group consisting of ¹⁸⁶Re, ^{99m}Tc, ¹¹¹In, ¹⁷⁷Lu, ⁸⁹Zr, ⁶⁴Cu, ⁶⁸Ga, ⁴⁴Sc, ⁶⁰Co, or ¹⁵³Gd.

More preferably, the tracer (T) is selected from the group consisting of

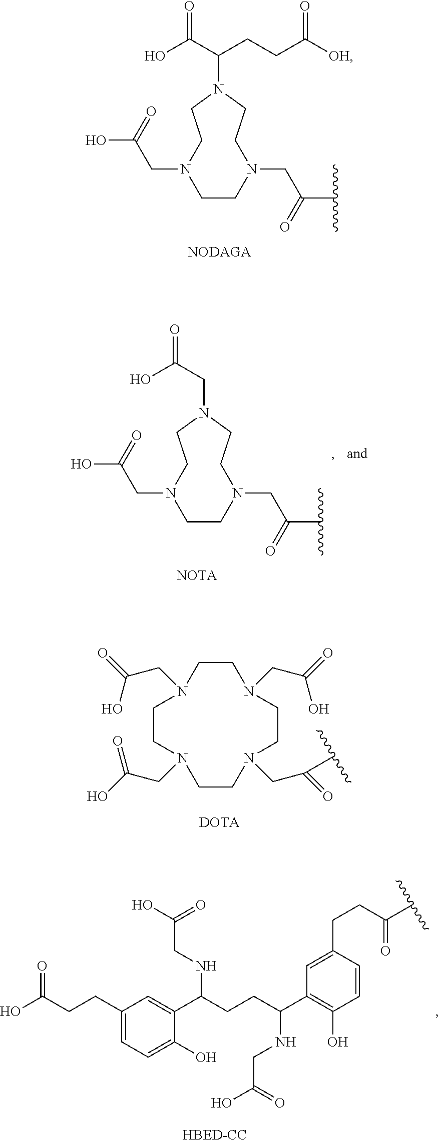

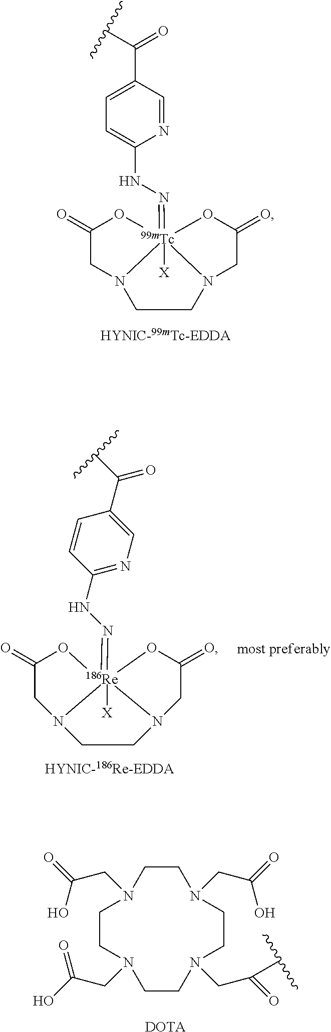

wherein X is a halogen, such as Cl, Br, F, I or water.

HBED-CC has been described in the literature, see J. Šimeček et al., Mol. Pharmaceutics 2014, 11, 11, 3893-3903, E. Boros et al., Nucl. Med. Biol. 2012, 29, 785-794, J. Zöller et al., J. Nucl. Med. 1992, 33, 1366-1372, R. Ferreirós-Martinez et al., Dalton Trans. 2008, 5754-5765.

Preferably,
a) NODAGA forms a chelate complex with a radioactive metal, preferably selected from the group consisting of ¹¹¹In, ¹⁷⁷Lu, ⁶⁴Cu, ⁶⁸Ga, ⁴⁴Sc and/or
b) NOTA forms a chelate complex with a radioactive metal, preferably selected from the group consisting of ¹¹¹In, ¹⁷⁷Lu, ⁶⁴Cu, ⁶⁸Ga, ⁴⁴Sc and/or
c) DOTA forms a chelate complex with a radioactive metal, preferably selected from the group consisting of ¹⁸⁶Re, ^{99m}Tc, ¹¹¹In ¹⁷⁷Lu, ⁸⁹Zr, ⁶⁴Cu, ⁶⁸Ga, ⁶⁰Co, ⁴⁴Sc and ¹⁵³Gd.
d) HBED-CC forms a chelate complex with a radioactive metal, preferably ⁶⁸Ga.
e) HYNIC-^{99m}Tc-EDDA and HYNIC-¹⁸⁶Re-EDDA forms chelate complexes with ^{99m}Tc and ¹⁸⁶Re.

The overall Linker (L) comprises the following parts connected as follows:

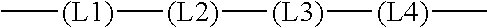

The (L1) end is connected to the DNA-binder (DNAB) via Y or Z, as defined below.
At least one or more (L4) is/are connected to a tracer (T)/tracer moieties (T). Wherein each respective (L4) is at most connected to one tracer (T). Optionally, one or more further (L4) may be present which are not connected with the tracer (T).
(L2) is optionally connected with more than one (L3) and/or more than one (L1). Each of (L3), if present, is connected to a (L4). Thus, (L2) may, for example, be connected with three (L3) and one (L1) or two (L3) and two (L1), or one (L1) and one (L3) or other combinations. However, (L2) is connected at least with one (L1) and one (L3).
(L1) is selected from the group consisting of

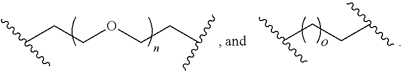

n is an integer between 1 and 10; preferably between 1 and 6, more preferably between 1 and 3, most preferably 3;
o is an integer between 1 and 10; preferably between 1 and 6, more preferably between 1 and 3, most preferably 3.
(L2) is selected from the group consisting of absent,

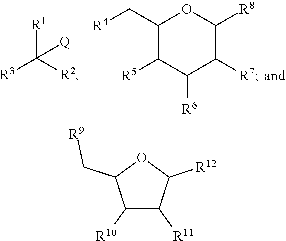

Q is selected from the group consisting of H, —NH—, and —NH₂, preferably —NH—.
R¹, R², R³, R⁴, R⁵, R⁶, R⁷, R⁸, R⁹, R¹⁰, R¹¹, and R¹² are independently selected from the group consisting of —(CH₂)ₚOH, —(CH₂)ₚO—, —(CH₂)ₚNH₂, —(CH₂)ₚNH—, —(CH₂)ₚSH, —(CH₂)ₚS—, —(CH₂)ₚPhOH, —(CH₂)ₚPhO—, —NH₂, —NHC(O)— or N(C₁-C₆)alkylC(O)—, —NH—, —COOH, —CONH—, —(CH₂)ₚOPO₃—, —(CH₂)ₚOSO₂—, —(CH₂)ₚOSO₂O—, —(CH₂)ₚSO₃H, —(CH₂)ₚOSO₂OH, —(CH₂)ₚOSO₂NH₂, —(CH₂)ₚOSO₂NH—,

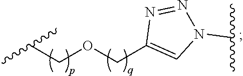

preferably

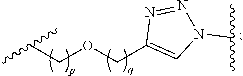

In (L2) at least two of Q, R¹, R², R³, R⁴, R⁵, R⁶, R⁷, R⁸, R⁹, R¹⁰, R¹¹, or R¹² are selected from the group consisting of —(CH₂)ₚO—, —(CH₂)ₚNH—, —(CH₂)ₚS—, —(CH₂)ₚPhO—, —NHC(O)— or N(C₁-C₆)alkylC(O)—, —NH—, —CONH—, —(CH₂)ₚOPO₃—, −(CH₂)ₚOSO₂—, —(CH₂)ₚOSO₂O—, —(CH₂)ₚOSO₂NH—,

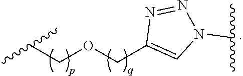

If any one of Q, R¹, R², R³ R⁴, R⁵, R⁶, R⁷, or R⁸, R⁹, R¹⁰, R¹¹, or R¹² is independently selected from the group consisting of —(CH₂)ₚOH, —(CH₂)ₚNH₂,—(CH₂)ₚSH,—(CH₂)ₚPhOH, —NH₂, —COOH, —(CH₂)ₚSO₃H, —(CH₂)ₚOSO₂OH, or —(CH₂)ₚOSO₂NH₂, then for the respective Q, R¹, R², R³, R⁴, R⁵, R⁶, R⁷, R⁸, R⁹, R¹⁰, R¹¹, or R¹² selected from this group, (L3) and (L4) are absent.
p is an integer between 0 and 10; preferably 1 and 3, more preferably 1.
q is an integer between 1 and 4, preferably 1 and 3, more preferably 1.
(L3) is selected from the group consisting of absent,

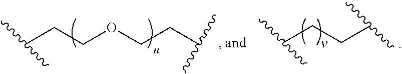

u is an integer between 1 and 10; preferably between 1 and 6, more preferably between 1 and 3, most preferably 3.
v is an integer between 1 and 10; preferably between 1 and 6, more preferably between 1 and 3, most preferably 3.
(L4) is independently selected from the group consisting of absent, —NH—, —(CH₂)ₛSO₃H, —(CH₂)ₛOH, —(CH₂)ₛNH₂, —(CH₂)ₛPhOH, —NH₂, —COOH, —(CH₂)ₛOSO₂OH, —(CH₂)ₛOSO₂NH₂, —(CH₂)ₛOSO₂NH—, preferably —NH—.
If the respective (L4) is independently selected from the group consisting of —NH—, and —(CH₂)ₛOSO₂NH— then (L4) is connected to a tracer (T).
s is an integer between 0 and 10; preferably 1 and 3, more preferably 1.

In one embodiment in the DNA-probe
a) the DNA-binder (DNAB) is selected from the group consisting of

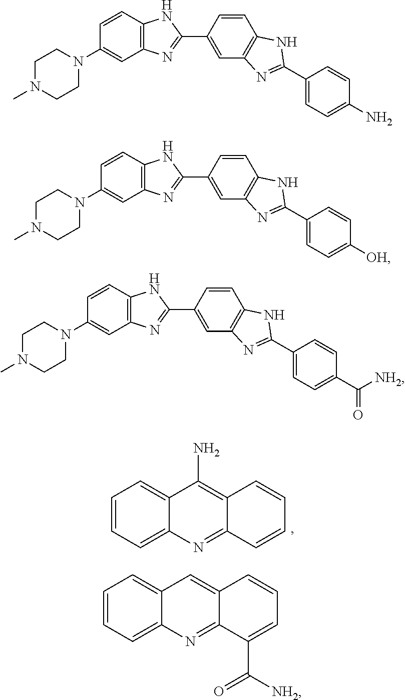

b) the tracer (T) is selected from the group consisting of

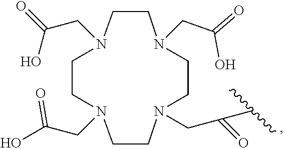 [SMILES: *C(=O)CN1CCN(CC(=O)O)CCN(CC(=O)O)CCN(CC(=O)O)CC1]

and/or
c) in the linker (L),
i) (L1) is

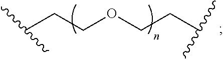

and/or
n is an integer between 1 and 10; preferably between 1 and 6, more preferably between 1 and 3, most preferably 3;
ii) (L2) is absent, or

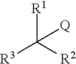

wherein Q is selected from the group consisting of absent, —NH—, —NH₂
R¹, R², or R³ are independently selected from the group consisting of —(CH₂)ₚOH, —(CH₂)ₚO—, —(CH₂)ₚNH₂, —(CH₂)ₚNH—, —(CH₂)ₚSH, —(CH₂)ₚS—, —(CH₂)ₚPhOH, —(CH₂)ₚPhO—, —NH₂, —NHC(O)— or N(C₁-C₆)alkylC(O)—, —NH—, —COOH, —CONH—, —(CH₂)ₚOPO₃—, —(CH₂)ₚOSO₂—, —(CH₂)ₚOSO₂O—, —(CH₂)ₚOSO₂OH, —(CH₂)ₚOSO₂NH₂, —(CH₂)ₚOSO₂NH—,

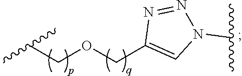

wherein in (L2) at least two of Q, R¹, R², or R³ are selected from the group consisting of —(CH₂)ₚO—, —(CH₂)ₚNH—, —(CH₂)ₚS—, —(CH₂)ₚPhO—, —NHC(O)— or N(C₁-C₆)alkylC(O)—, —NH—, —CONH—, —(CH₂)ₚOPO₃—, —(CH₂)ₚOSO₂—, —(CH₂)ₚOSO₂O—, —(CH₂)ₚOSO₂NH—,

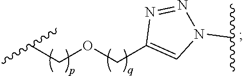

p is an integer between 0 and 10; preferably 1 and 3, more preferably 1;
q is an integer between 1 and 4, preferably 1 and 3, more preferably 1; and/or
iii) (L3) is absent, or

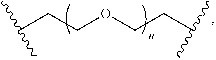

n is an integer between 1 and 10; preferably between 1 and 6, more preferably between 1 and 3, most preferably 3;
iv) (L4) is —NH—.

In one embodiment, the DNA-probe is selected from the group consisting of

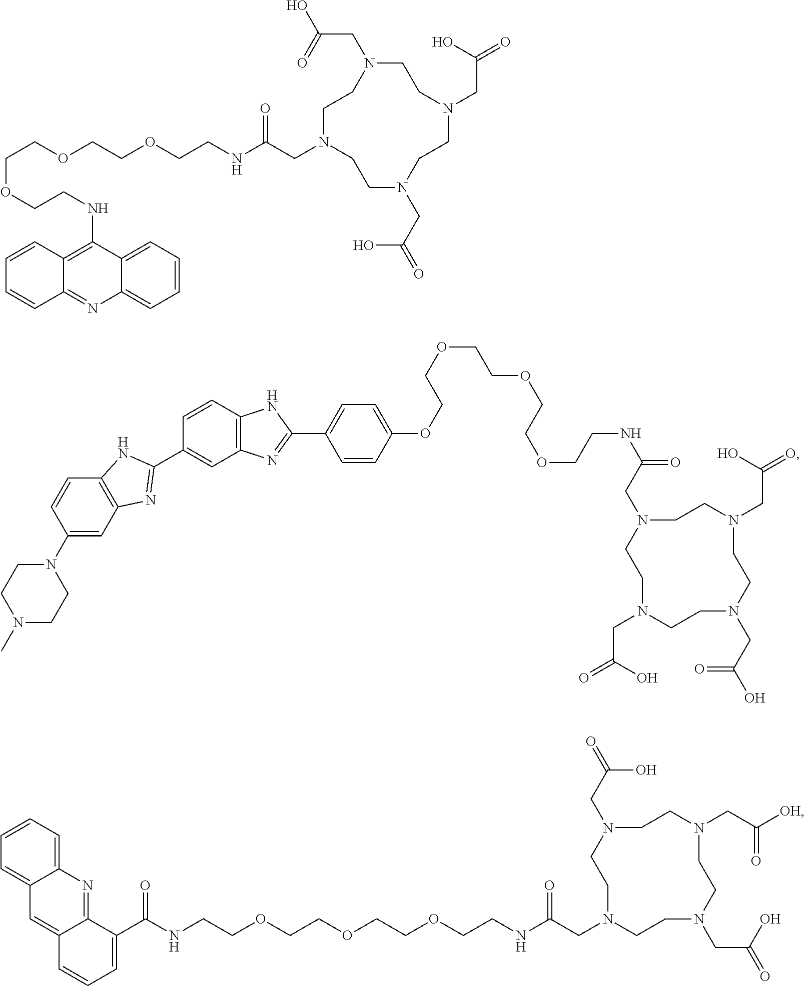 [SMILES: O=C(O)CN1CCN(CC(=O)O)CCN(CC(=O)NCCOCCOCCOCCNc2c3ccccc3nc3ccccc23)CCN(CC(=O)O)CC1 ; CN1CCN(c2ccc3[nH]c(-c4ccc5[nH]c(-c6ccc(OCCOCCOCCOCCNC(=O)CN7CCN(CC(=O)O)CCN(CC(=O)O)CCN(CC(=O)O)CC7)cc6)nc5c4)nc3c2)CC1 ; O=C(O)CN1CCN(CC(=O)O)CCN(CC(=O)NCCOCCOCCOCCNC(=O)c2cccc3cc4ccccc4nc23)CCN(CC(=O)O)CC1]

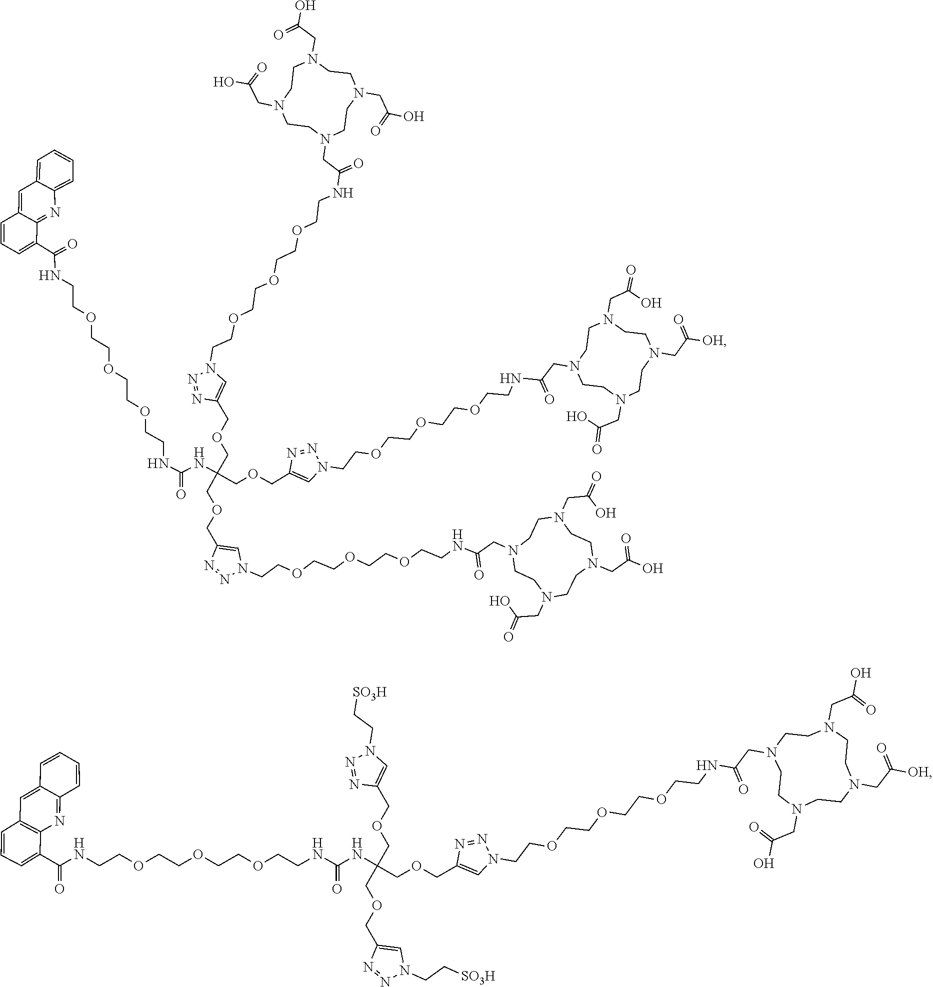 [SMILES: O=C(O)CN1CCN(CC(=O)O)CCN(CC(=O)NCCOCCOCCOCCn2cc(COCC(COCc3cn(CCOCCOCCOCCNC(=O)CN4CCN(CC(=O)O)CCN(CC(=O)O)CCN(CC(=O)O)CC4)nn3)(COCc3cn(CCOCCOCCOCCNC(=O)CN4CCN(CC(=O)O)CCN(CC(=O)O)CCN(CC(=O)O)CC4)nn3)NC(=O)NCCOCCOCCOCCNC(=O)c3cccc4cc5ccccc5nc34)nn2)CCN(CC(=O)O)CC1 ; O=C(O)CN1CCN(CC(=O)O)CCN(CC(=O)NCCOCCOCCOCCn2cc(COCC(COCc3cn(CCS(=O)(=O)O)nn3)(COCc3cn(CCS(=O)(=O)O)nn3)NC(=O)NCCOCCOCCOCCNC(=O)c3cccc4cc5ccccc5nc34)nn2)CCN(CC(=O)O)CC1]

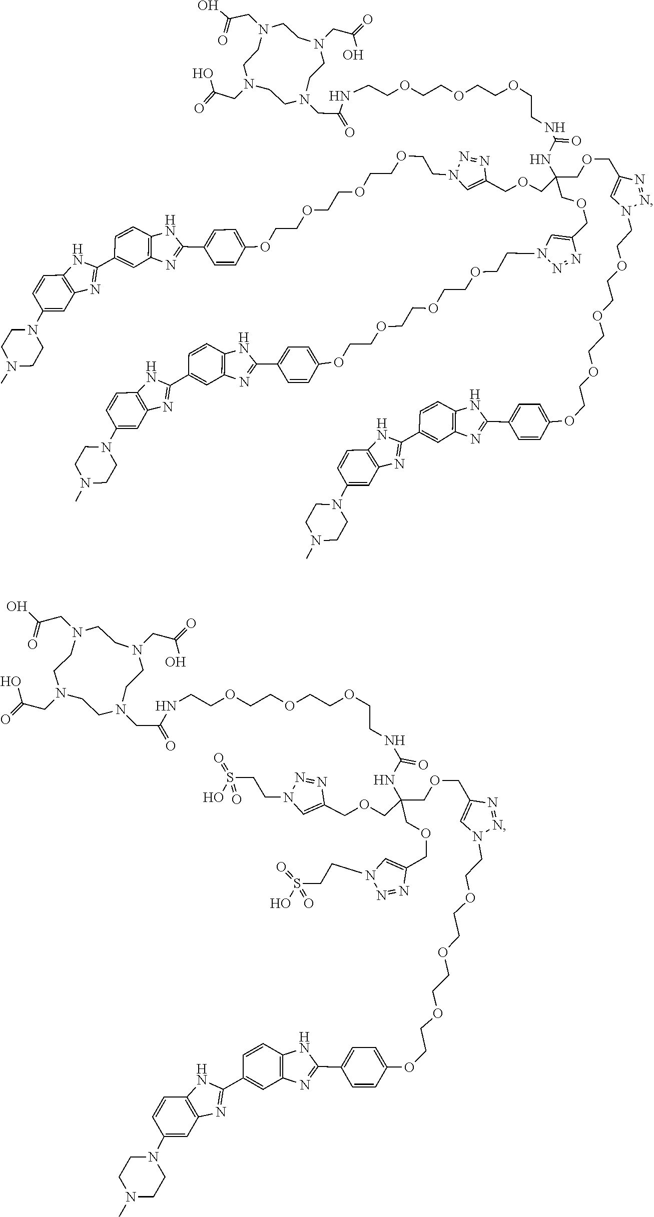

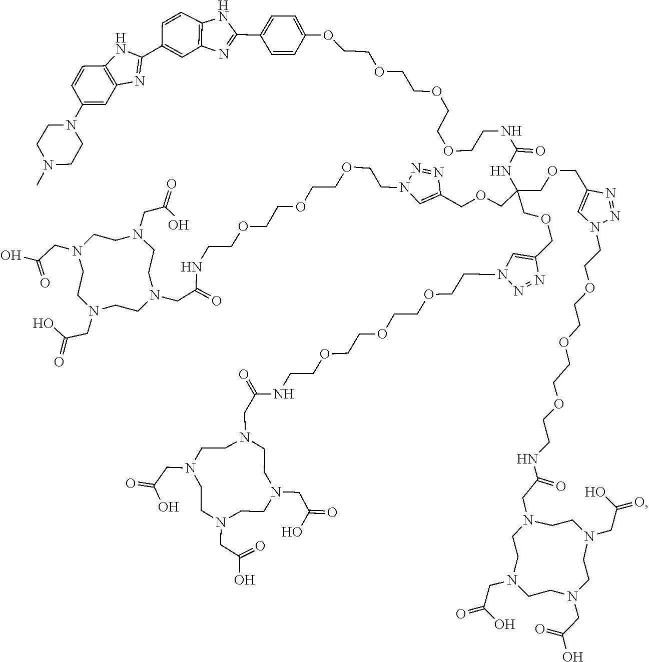 [SMILES: CN1CCN(c2ccc3[nH]c(-c4ccc5[nH]c(-c6ccc(OCCOCCOCCOCCNC(=O)NC(COCc7cn(CCOCCOCCOCCNC(=O)CN8CCN(CC(=O)O)CCN(CC(=O)O)CCN(CC(=O)O)CC8)nn7)(COCc7cn(CCOCCOCCOCCNC(=O)CN8CCN(CC(=O)O)CCN(CC(=O)O)CCN(CC(=O)O)CC8)nn7)COCc7cn(CCOCCOCCOCCNC(=O)CN8CCN(CC(=O)O)CCN(CC(=O)O)CCN(CC(=O)O)CC8)nn7)cc6)nc5c4)nc3c2)CC1]

or a chelate complex with a metal ion, preferably a radioactive metal ion thereof, more preferably selected from the group consisting of ¹⁸⁶Re, ^{99m}Tc, ¹¹¹In, ¹⁷⁷Lu, ⁸⁹Zr, ⁶⁴Cu, ⁶⁸Ga, ⁴⁴Sc, ⁶⁰Co, ¹⁵³Gd thereof.

In another embodiment, the DNA-probe is selected from the group consisting of

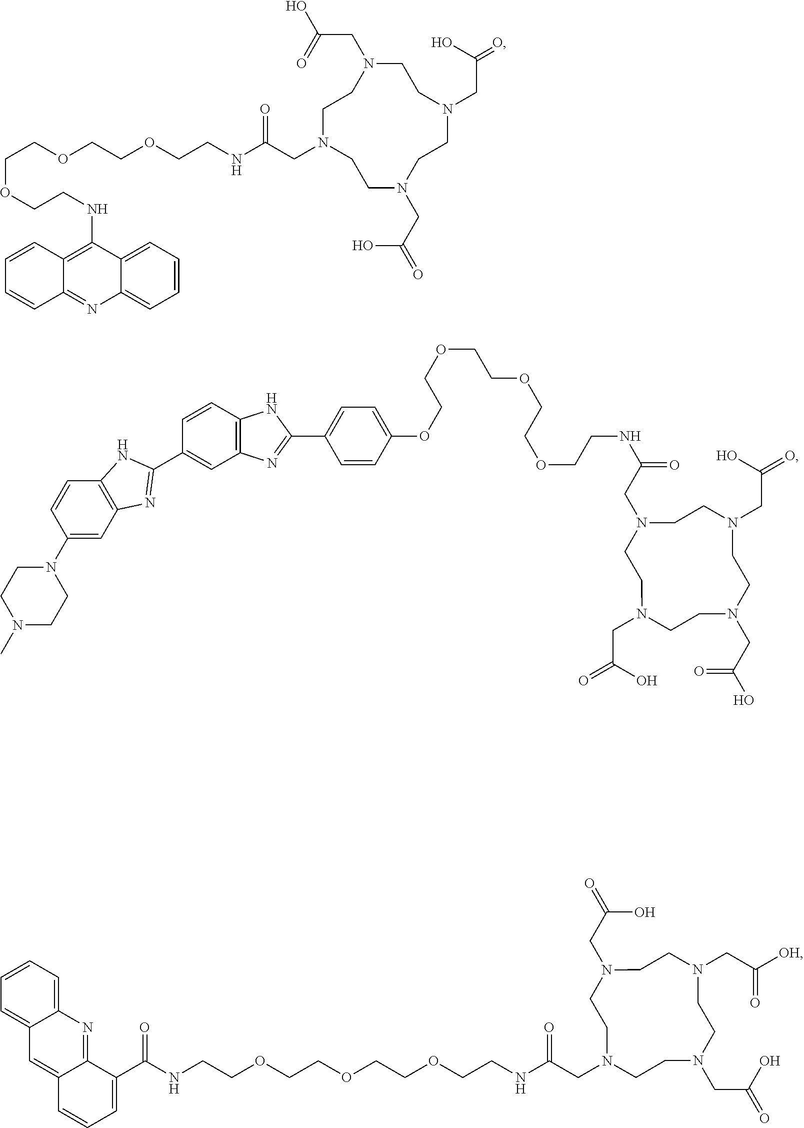 [SMILES: O=C(O)CN1CCN(CC(=O)O)CCN(CC(=O)NCCOCCOCCOCCNc2c3ccccc3nc3ccccc23)CCN(CC(=O)O)CC1 ; CN1CCN(c2ccc3[nH]c(-c4ccc5[nH]c(-c6ccc(OCCOCCOCCOCCNC(=O)CN7CCN(CC(=O)O)CCN(CC(=O)O)CCN(CC(=O)O)CC7)cc6)nc5c4)nc3c2)CC1 ; O=C(O)CN1CCN(CC(=O)O)CCN(CC(=O)NCCOCCOCCOCCNC(=O)c2cccc3cc4ccccc4nc23)CCN(CC(=O)O)CC1]

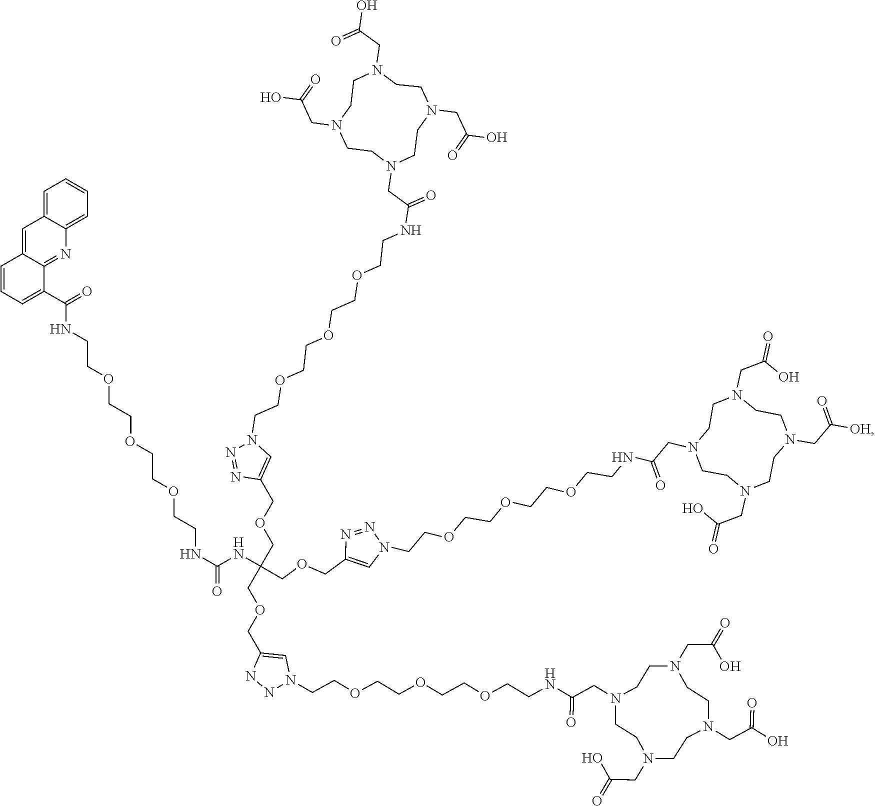 [SMILES: O=C(O)CN1CCN(CC(=O)O)CCN(CC(=O)NCCOCCOCCOCCN2C=C(COCC(COCc3cn(CCOCCOCCOCCNC(=O)CN4CCN(CC(=O)O)CCN(CC(=O)O)CCN(CC(=O)O)CC4)nn3)(COCc3cn(CCOCCOCCOCCNC(=O)CN4CCN(CC(=O)O)CCN(CC(=O)O)CCN(CC(=O)O)CC4)nn3)NC(=O)NCCOCCOCCOCCNC(=O)c3cccc4cc5ccccc5nc34)NN2)CCN(CC(=O)O)CC1]

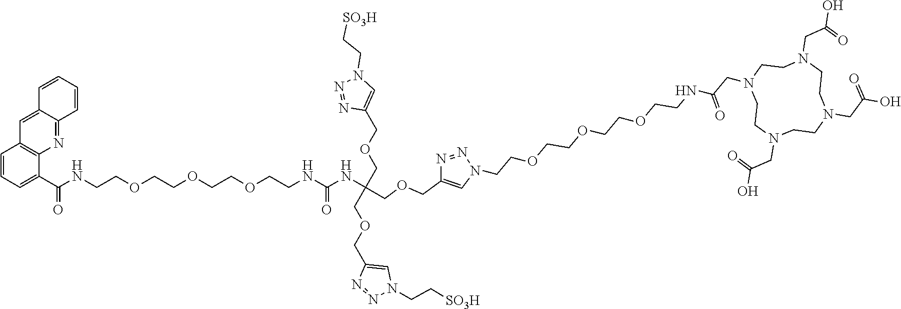 [SMILES: O=C(O)CN1CCN(CC(=O)O)CCN(CC(=O)NCCOCCOCCOCCn2cc(COCC(COCc3cn(CCS(=O)(=O)O)nn3)(COCc3cn(CCS(=O)(=O)O)nn3)NC(=O)NCCOCCOCCOCCNC(=O)c3cccc4cc5ccccc5nc34)nn2)CCN(CC(=O)O)CC1]

The DNA-probe is for use in detection of extracellular DNA of bacteria. The DNA-probe may be used in vitro or in vivo, preferably in vivo.

A better understanding of the present invention and of its advantages will be had from the following examples, offered for illustrative purposes only. The examples are not intended to limit the scope of the present invention in any way.

### EXAMPLES OF THE INVENTION

All non-aqueous reactions were carried out under dry Ar atmosphere by using Schlenk line techniques as standard. Commercially available chemicals and solvents were used without further purification unless otherwise described and were purchased from Sigma-Aldrich, Acros Organics, Alfa Aesar and Fluorochem. Deuterated solvents were purchased from Deutero GmbH. All dyes used were purchased from Lumiprobe GmbH. For moisture-sensitive NMR samples, solvents were dried over AlOx.

CHROMAFIL® Xtra PTFE-45/25 syringe filters from Macherey-Nagel with a pore size of 0.45 μm were used to filter solutions prior to automated chromatographic purification.

THF was dried over Na/benzophenone and distilled under Ar atmosphere. NEt₃ was distilled over KOH and DIPEA over CaH₂ under N₂ atmosphere.

Molecular sieve was activated by heating to 160° C. in high vacuum overnight before use.

Ice-salt mixtures for −20° C. and dry ice-acetone mixtures for −78° C. were used to cool reactions.

Organic solvents were removed on a rotary evaporator at a reduced temperature of 45° C., unless otherwise stated. Aqueous solutions were concentrated by freeze-drying using an Alpha 2-4 freeze-drying analogue from Christ.

All NMR experiments were performed with a Bruker spectrometer type DPX-400 (¹H: 400.13 MHz, ¹³C{1H}: 100.61 MHz)), type DPX-500 (¹H: 500.13 MHz, ¹³C{1H}: 125.76 MHz)) and type DPX-600 (1H: 600.33 MHz, ¹³C{1H}: 150.95 MHz)) at room temperature in the indicated deuterated solvents. The chemical shift 5 is given in ppm relative to Si(CH3)4. All samples for the 1H-spectroscopy contain the residual proton signal of the respective solvent as reference (chloroform: δ (1H-NMR)=7.26 ppm, methanol: δ (¹H-NMR)=3.31 ppm, dimethyl sulphoxide: δ (1H-NMR)=2.50 ppm, acetone: δ (1H-NMR)=2.05 ppm, water: δ (¹H-NMR)=4.79 ppm). All samples for 13C spectroscopy were referenced to the solvent signal and were recorded broadband decoupled (chloroform: δ (¹³C-NMR)=77.2 ppm, methanol: δ (¹³C-NMR)=49.0 ppm, dimethyl sulphoxide: δ (¹H-NMR)=39.5 ppm). The coupling constants J are given in Hz and the corresponding signal multiplicities are abbreviated as follows:
Singlet (s), Duplet (d), Triplet (t), Quartet (q) and Multiplet (m). TOPSPIN was used to process and evaluate the spectra.
High resolution mass spectrometry (HRMS) was performed on a Waters Micromass LCT-Premier spectrometer. A Lockspray Dual Ion Source and a Waters Alliance 2695 system were used. Ionisation was performed by electrospray ionisation (ESI). All values are given in mass/charge (m/z).

LC-MS data for reaction controls were measured as indicated for the respective compounds either on an Agilent 1100 series HPLC system coupled to an Esquire 3000plus MS detector using C8 HPLC columns.

### Methods:

DEFAULT.M (ESI-MS+): 0 min, 97% H₂O/3% MeCN (0.05% TFA); 1.5 min, 97% MeCN/3% H₂O (0.05% TFA); 2 min, running end; 1 ml/min; 30° C.
DEFAULTUNPOL.M (ESI-MS+): 0 min, 97% H₂O/3% MeCN (0.05% TFA); 1 min, 97% MeCN/3% H₂O (0.05% TFA); 2 min, running end; 1 ml/min; 30° C.

Column chromatography was performed under overpressure with the indicated solvent mixtures manually or automatically with a Reveleris Prep. system from Buchi and was carried out either with silica gel from Macherey-Nagel (particle size: 40-63 μm, normal phase) or with C18-modified silica gel from Macherey-Nagel (particle size: 40-63 μm, reversed phase) as stationary phases. For normal phase column chromatography, the crude product was either applied as a solution in the starting eluent mixture or immobilised on Celite. For reversed-phase column chromatography, the crude product was applied as a solution in the starting eluent mixture and running media containing 0.05% TFA were used.

High-pressure liquid chromatography (HPLC) for the analysis of the radiolabeling of probe 50 was performed on a LaChrome 7000 series from Merck using a Luna C18 LC column (250 mm×4.6 mm) from Phenomenex with the gradient: 0 min, 99% H₂O/1% MeCN (0.05% TFA); 30 min, 99% MeCN/1% H₂O (0.05% TFA); 30 min, 1 ml/min; 30° C.

The compounds shown below were synthesized to according literature

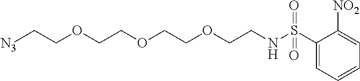 [SMILES: [N-]=[N+]=NCCOCCOCCOCCNS(=O)(=O)c1ccccc1[N+](=O)[O-]]

### N-(2-(2-(2-(2-azidoethoxy)ethoxy)ethoxy)ethyl)-2-nitrobenzenesulfonamide

### N₃-PEG4-NHNs^{[1]}

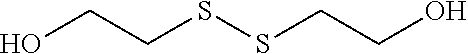

### 2,2′-disulfanediylbis(ethan-1-ol)

### N₃/OH-disulfide^{[13]}

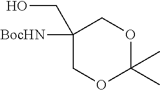 [SMILES: CC(C)(C)OC(=O)NC1(CO)COC(C)(C)OC1]

### Tert-Butyl (5-(hydroxymethyl)-2,2-dimethyl-1,3-dioxan-5-yl)carbamate 1

Tris (5.0 g, 41.3 mmol) was dissolved in CH₃OH (200 ml) at room temperature. A solution of Boc₂O (9.9 g, 45.5 mmol) in CH₃OH (50 ml) was added to the solution. The reaction was stirred for 18 h and subsequently the solvent was removed in vacuo. The obtained crude product was taken up at room temperature in CH₂Cl₂ (200 ml). To the suspension were added 2,2-dimethoxypropane (130 ml, 123.8 mmol) and p-toluenesulfonic acid (355 mg, 2.1 mmol), successively. The reaction was stirred for 2 h. The reaction was stopped by adding triethylamine (350 μl). The solvents were removed in vacuo and the crude product was purified by column chromatography (PE/EE=1/1). 1 (8645 mg, 39.1 mmol, 95%) was isolated as a white solid.

¹H NMR (MHz, CDCl₃): δ=5.35 (s, 1H), 3.84 (q, J=11.8 Hz, 4H), 3.74 (s, 2H), 1.47 (bs, 16H).

The spectroscopic data correspond to the literature ^{[2]}

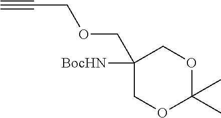 [SMILES: C#CCOCC1(NC(=O)OC(C)(C)C)COC(C)(C)OC1]

### Tert-Butyl (2,2-dimethyl-5-((prop-2-yn-1-yloxy)methyl)-1,3-dioxan-5-yl)carbamate 2

1 (5.8 g, 22.3 mmol) was dissolved in THF (35 ml) at 0° C. NaI (500 mg, 3.3 mmol), TBAI (82 mg, 1 mol %) and propargyl bromide (80% w/w in PhMe, 2.9 ml, 26.7 mmol) were added to the solution. Subsequently, KOH (2.5 g, 44.5 mmol) was added to the solution in portions over a period of 30 minutes. The resulting suspension was stirred at 37° C. for 2 h. The reaction was stopped by adding water. The reaction mixture was concentrated in vacuo and the residue was taken up in water and EtOAc. The aqueous phase was extracted with EtOAc. The combined organic phases were dried over Na₂SO₄, filtered and the solvent was removed in vacuo. The crude product was purified by column chromatography (PE/EA=96/4-3/1). 2 (5.5 g, 18.5 mmol, 83%) was isolated as a yellow oil.

¹H NMR (MHz, CDCl₃): δ=4.89 (s, 1H), 4.20 (d, J=2.4, 4H), 3.83 (s, 5H), 2.47 (s, 1H), 1.52 (s, 3H), 1.48 (s, 9H), 1.45 (s, 9H), 1.42 (s, 3H).

¹³C NMR (MHz, CDCl₃): δ=154.8, 98.4, 79.3, 74.8, 69.7, 64.3, 62.7, 59.2, 58.8, 58.7, 51.8, 30.9, 28.4, 24.6, 22.8

R_{f} (PE/EA=5/1): 0.35

HRMS (ESI): m/z calculated for C₁₅H₂₆NO₅[M+H]⁺: 300.1811, found: 300.1816

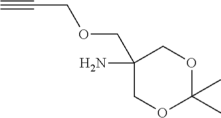

### 2,2-dimethyl-5-((prop-2-yn-1-yloxy)methyl)-1,3-dioxan-5-amine 3

2 (165 mg, 0.6 mmol) was dissolved in CH₂Cl₂ (4.1 ml) and cooled to −78° C. To this solution was added 2.6 lutidine (96 μl, 0.8 mmol) and TMSOTf (130 μl, 0.7 mmol). After complete addition, the reaction solution was warmed to 0° C. and was allowed to stir at this temperature for 2 h. The reaction was stopped by adding saturated aq. NaHCO₃ solution. The aqueous phase was extracted with EtOAc. The combined organic phases were dried over Na₂SO₄, filtered and the solvent was removed in vacuo. The crude product was purified by column chromatography (CH₃OH in CH₂Cl₂: 6%-10%). 3 (85 mg, 0.4 mmol, 77%) was isolated as a yellow oil.

¹H NMR (MHz, CDCl₃): δ=4.20 (d, J=2.3 Hz, 2H), 3.84 (d, J=11.6 Hz, 2H), 3.58-3.52 (m, 4H), 2.46 (t, J=2.4, 1H), 1.73 (s, 2H), 1.46 (s, 3H), 1.43 (s, 3H).

¹³C NMR (MHz, CDCl₃): δ=98.2, 79.4, 74.7, 72.7, 67.5, 67.2, 58.8, 49.4, 48.6, 23.7, 23.4.

R_{f} (10% CH₃OH in CH₂Cl₂): 0.3

HRMS (ESI): m/z calculated for C₁₀H₁₈NO₃[M+H]+: 200.1287, found: 200.1284

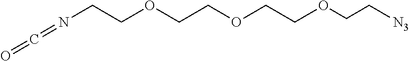 [SMILES: [N-]=[N+]=NCCOCCOCCOCCN=C=O]

### 1-azido-2-(2-(2-isocyanatomethoxy)ethoxy)ethoxy)ethane

Triphosgene (2.1 g, 7.2 mmol) was dissolved in CH₂Cl₂ (15 ml) at room temperature. To the resulting solution were added solutions of triethylamine (1.5 ml, 11.1 mmol) in CH₂Cl₂ (15 ml) and 16 (1.6 g, 4.8 mmol) in CH₂Cl₂ (15 ml), successively. The reaction was stirred for 2 h at room temperature. The reaction was stopped by adding saturated NaHCO₃ solution. The aqueous phase was extracted with CH₂Cl₂. The combined organic phases were dried over Na₂SO₄, filtered and the solvent was removed in vacuo. The crude product obtained (2658 mg, quantitative yield) was isolated as a yellow oil and used without further purification steps.

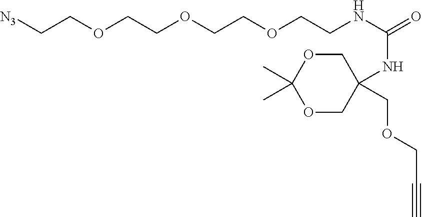 [SMILES: C#CCOCC1(NC(=O)NCCOCCOCCOCCN=[N+]=[N-])COC(C)(C)OC1]

### 1-(2-(2-(2-(2-azidoethoxy)ethoxy)ethoxy)ethyl)-3-(2,2-dimethyl-5-((prop-2-yn-1-yloxy)methyl)-1,3-dioxan-5-yl)urea

4 (amine, 3.3 g, 16.5 mmol) was placed at room temperature. A solution of 3 (4.4 g, 18.2 mmol) and triethylamine (3.4 ml, 24.8 mmol) in CH₂Cl₂ (165 ml) was added to the reaction. The resulting solution was stirred for 72 h. Subsequently, the reaction solution was concentrated in vacuo and the crude product was purified by column chromatography (PE/EA=1/6). 5 (1.2 g, 2.8 mmol, 17%) was isolated as a yellow oil.

¹H NMR (400 MHz, CDCl₃) δ=5.04 (s, 1H), 4.29 (s, 1H), 4.26 (s, 1H), 4.20 (d, J=2.4 Hz, 2H), 3.85 (s, 2H), 3.73-3.65 (m, 11H), 3.59-3.55 (t, J=5.3 Hz, 2H), 3.45 (t, J=5.6 Hz, 4.6, 2H), 3.36 (t, J=5.6 Hz, 2H), 2.47 (t, J=2.4 Hz, 1H), 1.52 (s, 3H), 1.42 (s, 3H).

¹³C NMR (400 MHz, CDCl₃) δ=157.7, 98.6, 79.5, 74.8, 70.7, 70.56, 70.4, 70.2, 70.0, 64.7, 58.7, 52.5, 50.7, 40.9, 40.2, 30.9, 24.7, 23.9, 22.8.

HRMS (ESI): m/z calculated for C₁₉H₃₃N₅O₇Na[M+Na]⁺: 466.2278, found: 466.2281

R_{f} (10% CH₃OH in CH₂Cl₂): 0.5

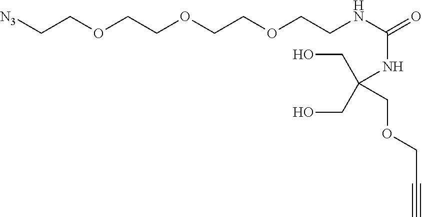 [SMILES: C#CCOCC(CO)(CO)NC(=O)NCCOCCOCCOCCN=[N+]=[N-]]

### 1-(2-(2-(2-(2-azidoethoxy)ethoxy)ethoxy)ethyl)-3-(1,3-dihydroxy-2-((prop-2-yn-1-yloxy)methyl)propan-2-yl)urea

5 (1.2 g, 2.8 mmol) was dissolved at 4° C. in AcOH/1 M HCl (9/1, 34 ml). The solution was stirred for 1 h. Subsequently, the reaction was stopped by adding a saturated NaHCO₃ solution. The aqueous phase was extracted with EtOAc, the combined organic phases were dried over Na₂SO₄, filtered and the solvent was removed under reduced pressure. 6 (985 mg, 2.4 mmol, 89%) was used without further purification steps.

¹H NMR (400 MHz, CDCl₃): δ=5.62 (s, 1H), 4.21 (d, J=2.3 Hz, 2H), 3.79 (s, 1H), 3.76 (s, 1H), 3.71-3.75 (m, 8H), 3.67 (s, 4H), 3.59 (t, J=5.5 Hz, 2H), 3.56 (s, 1H), 3.53 (s, 1H), 3.45 (t, J=5.1 Hz, 2h), 3.38 (t, J=5.1 Hz, 2H) 2.50 (t, J=2.4 Hz, 1H).

¹³C NMR (101 MHz, CDCl₃): δ=159.0, 79.1, 75.2, 71.1, 70.7, 70.5, 70.4, 70.3, 70.2, 70.0, 64.7, 59.9, 58.9, 50.7, 40.5, 23.8.

HRMS (ESI): m/z calculated for C₁₆H₃₀N₅O₇[M+H]⁺: 404.2145, found: 404.2140

R_{f} (10% CH₃OH in CH₂Cl₂): 0.45

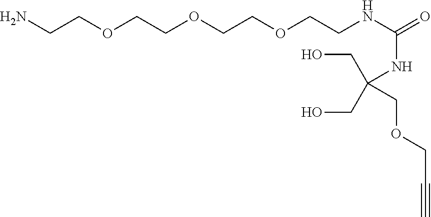 [SMILES: C#CCOCC(CO)(CO)NC(=O)NCCOCCOCCOCCN]

### 1-(2-(2-(2-(2-aminoethoxy)ethoxy)ethoxy)ethyl)-3-(1,3-dihydroxy-2-((prop-2-yn-1-yloxy)methyl)propan-2-yl)urea 7

6 (985 mg, 2.4 mmol) was dissolved in THF/H₂O (24 ml, 23/1) at room temperature. PMe₃ (1 M in THF, 5.6 ml, 5.6 mmol) was added to the solution and the reaction was stirred for 4 hours. Subsequently, the solvents were removed in vacuo and 7 (1169 mg, quantitative conversion) was obtained as a yellow oil and used without further purification steps.

¹H NMR (400 MHz, CDCl₃): δ=6.35 (s, 1H), 5.93 (s, 1H), 4.19 (d, J=2.4, 2H), 3.78 (s, 1H), 3.75 (s, 1H), 3.72-3.64 (m, 10H), 3.58 (m, 6H), 3.37 (q, J=5.1 Hz, 2H), 2.91 (t, J=5.0 Hz, 2H), 2.47 (t, J=2.4 Hz, 1H).

¹³C NMR (101 MHz, CDCl₃): δ=154.8, 98.4, 79.3, 74.8, 69.7, 64.3, 62.8, 59.2, 58.8, 58.7, 51.8, 30.9, 28.4, 24.6, 22.8.

HRMS (ESI): m/z calculated for C₁₆H₃₂N₃O₇[M+H]⁺: 378.2240, found: 378.2243

R_{f} (10% CH₃OH in CH₂Cl₂): 0.02

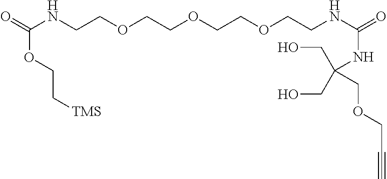 [SMILES: C#CCOCC(CO)(CO)NC(=O)NCCOCCOCCOCCNC(=O)OCC[Si](C)(C)C]

### 2-(trimethylsilyl)ethyl (15,15-bis(hydroxymethyl)-13-oxo-3,6,9,17-tetraoxa-12,14-diazaicos-19-yn-1-yl)carbamate 8

7 (922 mg, 2.4 mmol) was dissolved in CH₂Cl₂ (25 ml) at room temperature. Triethylamine (510 μl, 3.7 mmol) and Teoc-OSu (950 mg, 3.6 mmol) were added to the solution and the reaction was allowed to stir for 2.5 h. The reaction was stopped by adding a saturated Na₂CO₃ solution. The aqueous phase was extracted with CH₂Cl₂ and the combined organic phases were dried over Na₂SO₄, filtered and the solvent was removed under reduced pressure. The crude product was purified by column chromatography (CH₃OH in CH₂Cl₂: 3%). 8 (1041 mg, 2.0 mmol, 82%) was obtained as a yellow oil.

¹H NMR (400 MHz, CDCl₃): δ=5.79 (s, 1H), 5.48 (s, 1H), 4.20 (d, J=2.4 Hz, 2H), 4.15 (t, J=9.5 Hz, 1H), 3.79 (s, 1H), 3.76 (s, 1H), 3.71 (s, 2H), 3.67 (d, J=6.7 Hz, 8H), 3.63-3.57 (m, 5H), 3.55 (s, 1H), 3.38 (t, J=4.9 Hz, 4H), 2.49 (t, J=2.4 Hz, 1H), 1.07-0.96 (m, 2H), 0.06 (s, 6H).

¹³C NMR (400 MHz, CDCl₃): δ=171.8, 171.8, 75.1, 71.1, 70.3, 70.1, 70.1, 70.0, 64.7, 60.0, 58.9, 53.4, 40.7, 40.4, 25.4, 17.8, −1.5

HRMS (ESI): m/z calculated for C₂₂H₄₄N₃O₉Si[M+H]⁺: 522.2847, found: 522.2850

R_{f} (8% CH₃OH in CH₂Cl₂): 0.4

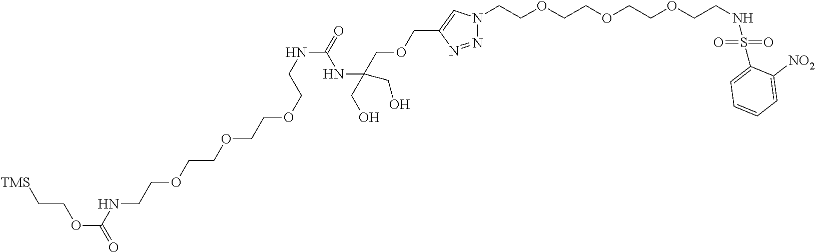 [SMILES: C[Si](C)(C)CCOC(=O)NCCOCCOCCOCCNC(=O)NC(CO)(CO)COCc1cn(CCOCCOCCOCCNS(=O)(=O)c2ccccc2[N+](=O)[O-])nn1]

### 2-(trimethylsilyl)ethyl (4,4-bis(hydroxymethyl)-1-(1-(2-(2-(2-(2-((2-nitrophenyl)sulfonamido)ethoxy)ethoxy)ethoxy)ethyl)-1H-1,2,3-triazol-4-yl)-6-oxo-2,10,13,16-tetraoxa-5,7-diazaoctadecan-18-yl)carbamate 9

8 (1.0 g, 2.0 mmol), 19 (0.8 g, 2.0 mmol), THPTA (86 mg, 10 mol %) and CuSO₄*5 H₂O (50 mg, 10 mol %) were dissolved in THF/H₂O (2/1, 20 ml) at room temperature. Sodium ascorbate (79 mg, 20 mol %) was added to the solution and the reaction was allowed to stir for 18 h. Subsequently, the solvents were removed in vacuo and the residue was taken up in CH₂Cl₂ and H₂O. The aqueous phase was extracted with CH₂Cl₂ and the combined organic phases were dried over Na₂SO₄, filtered and the solvent was removed under reduced pressure. The crude product was purified by column chromatography (CH₃OH in CH₂Cl₂: 4%-6%). 9 (1784 mg, 1.9 mmol, 97%) was isolated as a yellow oil.

¹H NMR (400 MHz, CDCl₃): δ=8.18-8.09 (m, 1H), 7.92-7.82 (m, 1H), 7.80 (s, 1H), 7.80-7.70 (m, 2H), 6.24 (t, J=5.8 Hz, 1H), 5.82 (s, 1H), 5.51 (s, 1H), 4.66 (s, 2H), 4.57 (t, J=5.0 Hz, 2H), 4.14 (t, J=8.6 Hz, 2H), 3.90 (t, J=5.0 Hz, 3H), 3.74 (s, 1H), 3.71 (s, 1H), 3.69 (s, 2H), 3.66-3.61 (m, 12H), 3.60-3.52 (m, 11H), 3.51 (s, 1H), 3.36-3.33 (m, 5H), 3.28 (q, J=5.6 Hz, 2H), 1.00-0.95 (m, 2H), 0.04 (s, 9H).

¹³C NMR (400 MHz, CDCl₃): δ=159.2, 157.0, 148.0, 144.2, 133.8, 133.6, 132.7, 131.0, 125.3, 123.8, 70.8, 70.5, 70.4, 70.4, 70.3, 70.3, 70.1, 70.1, 70.0, 69.4, 69.1, 64.5, 64.4, 63.0, 60.2, 53.5, 50.3, 43.5, 40.2, 17.8, −1.57.

HRMS (ESI): m/z calculated for C₃₆H₆₄N₈O₁₆SSiNa[M+Na]⁺: 947.3828, found: 947.3830

R_{f} (10% CH₃OH in CH₂Cl₂): 0.5

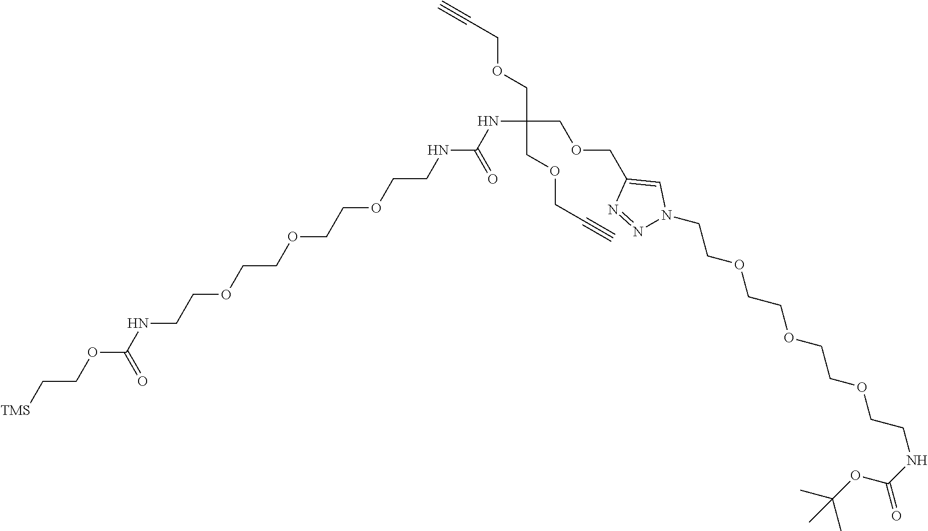 [SMILES: C#CCOCC(COCC#C)(COCc1cn(CCOCCOCCOCCNC(=O)OC(C)(C)C)nn1)NC(=O)NCCOCCOCCOCCNC(=O)OCC[Si](C)(C)C]

### 2-(trimethylsilyl)ethyl (15-(((1-(2,2-dimethyl-4-oxo-3,8,11,14-tetraoxa-5-azahexadecan-16-yl)-1H-1,2,3-triazol-4-yl)methoxy)methyl)-13-oxo-15-((prop-2-yn-1-yloxy)methyl)-3,6,9,17-tetraoxa-12,14-diazaicos-19-yn-1-yl)carbamate

9 (702 mg, 0.8 mmol) and Cs₂CO₃ (370 mg, 1.1 mmol) were placed at 0° C. in CH₃CN (15 ml). Thiophenol (116 μl, 1.14 mmol) was added dropwise to the suspension and the reaction was allowed to stir for 1.5 h while warming to room temperature. The reaction solution was then filtered through Celite and the solvent was removed in vacuo. The resulting residue (350 mg, 0.5 mmol) was taken up in CH₂Cl₂ (8 ml) and Boc₂O (113 μl, 0.5 mmol) and triethylamine (55 μl, 0.6 mmol) were added to the solution. The resulting solution was allowed to stir at room temperature for 1.5 h and subsequently all volatiles were removed in vacuo. The residue (380 mg, 0.45 mmol) was taken up in THF (650 μl) and cooled to 0° C. NaI (18 mg, 30 mol %) TBAI (3 mg, 1 mol %) and propargyl bromide (80% w/w in PhMe, 108 μl, 1.0 mmol) were added to the solution. Powdered KOH (90 mg, 1.6 mmol) was then added to the reaction over a period of 20 minutes. The resulting suspension was heated to 37° C. and stirred for 15 h. The reaction was stopped by adding water and the reaction solution was concentrated in vacuo. The residue was taken up in water and EtOAc and the aqueous phase was extracted with EtOAc. The combined organic phases were dried over Na₂SO₄, filtered and the solvent was removed in vacuo. The crude product was purified by column chromatography (CH₃OH in CH₂Cl₂: 2%-4%-6%). 10 (200 mg, 0.2 mmol, 18% over three steps) was isolated as a yellow oil.

¹H NMR (400 MHz, CDCl₃): δ=7.77 (s, 1H), 5.54 (s, 1H), 5.12 (s, 1H), 4.67 (s, 2H), 4.57 (t, J=5.1 Hz, 2H), 4.15 (d, J=2.4 Hz, 4H), 3.90 (t, J=5.1 Hz, 2H), 3.85 (s, 3H), 3.82 (s, 2H), 3.66 (s, 3H), 3.63 (d, J=5.3 Hz, 13H), 3.60-3.52 (m, 6H), 3.38 (d, J=4.7 Hz, 2H), 3.33 (t, J=5.4 Hz, 4H), 2.46 (t, J=2.3 Hz, 2H), 1.46 (s, 9H), 1.07-0.94 (m, 2H), 0.05 (s, 9H).

¹³C NMR (101 MHz, CDCl₃): δ=157.8, 157.8, 123.8, 79.8, 74.6, 70.6, 70.5, 70.46, 70.4, 70.4, 70.3, 70.3, 70.2, 70.1, 70.1, 70.1, 70.0, 69.9, 69.5, 69.5, 64.9, 62.9, 58.6, 53.4, 50.3, 40.7, 40.3, 40.0, 28.4, 17.8, −1.5.

HRMS (ESI): m/z calculated for C₃₅H₇₄N₇O₁₄Si[M+H]+: 840.4750, found: 840.4754

R_{f} (10% CH₃OH in CH₂Cl₂): 0.5

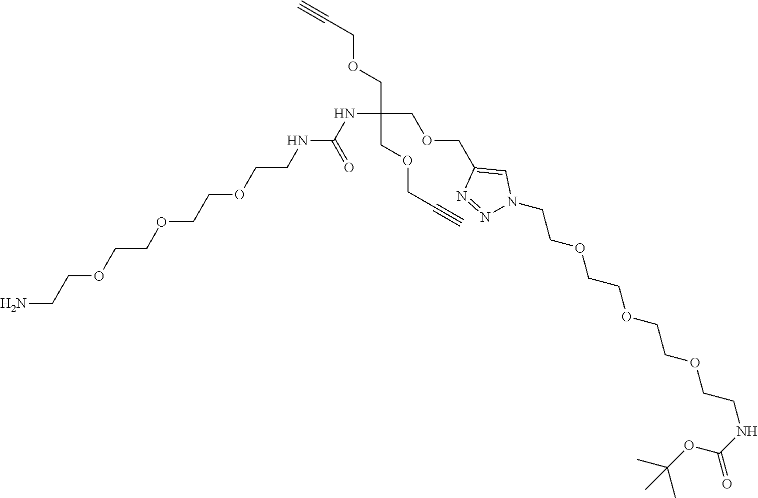 [SMILES: C#CCOCC(COCC#C)(COCc1cn(CCOCCOCCOCCNC(=O)OC(C)(C)C)nn1)NC(=O)NCCOCCOCCOCCN]

### Tert-Butyl (2-(2-(2-(2-(4-(18-amino-6-oxo-4,4-bis((prop-2-yn-1-yloxy)methyl)-2,10,13,16-tetraoxa-5,7-diazaoctadecyl)-1H-1,2,3-triazol-1-yl)ethoxy)ethoxy)ethoxy)ethyl)carbamate 11

10 (103 mg, 0.1 mmol) was placed in a Teflon flask. The flask was cooled to 0° C. and TASF (1M in DMF, 500 μl, 0.5 mmol) and H₂O (1M in DMF, 500 μl, 0.5 mmol) were added to the reaction. The reaction was then stirred for 5 days and warmed to room temperature. The solvents were removed in vacuo as far as possible. The crude product was taken up in CH₂Cl₂ and was purified by column chromatography (CH₃OH in CH₂Cl₂: 0%-10%-15%). 11 (75 mg, 0.09 mmol, 86%) was isolated as a yellow oil.

¹H NMR (400 MHz, CDCl₃): δ=7.77 (s, 1H), 4.67 (s, 1H), 4.66 (s, 2H), 4.55 (t, J=5.2 Hz, 2H), 4.16 (s, 1H), 4.14 (d, J=2.4 Hz, 2H), 3.89 (t, J=5.2 Hz, 3H), 3.84 (s, 3H), 3.80 (s, 2H), 3.61 (d, J=5.6 Hz, 18H), 3.56-3.48 (m, 8H), 3.37-3.29 (m, 6H), 2.84 (t, J=5.1 Hz, 2H), 2.45 (t, J=2.3 Hz, 2H), 1.44 (s, 9H).

¹³C NMR (101 MHz, CDCl₃): δ=157.9, 145.0, 123.8, 79.9, 74.5, 73.3, 70.6, 70.5, 70.5, 70.4, 70.4, 70.2, 70.1, 69.9, 69.7, 69.6, 69.5, 65.0, 58.6, 58.4, 53.5, 50.2, 41.3, 40.2, 39.7, 38.8.

HRMS (ESI): m/z calculated for C₃₅H₆₂N₇O₁₂[M+H]⁺: 772.4378, found: 772.4376

R_{f} (15% CH₃OH in CH₂Cl₂): 0.15

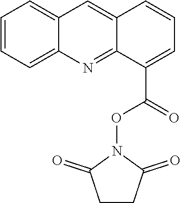 [SMILES: O=C(ON1C(=O)CCC1=O)c1cccc2cc3ccccc3nc12]

### 2,5-dioxopyrrolidin-1-yl acridine-4-carboxylate 12

Acridine-4-carboxylic acid 31 (72 mg, 0.32 mmol) was dissolved in CH₂Cl₂ at room temperature. DIPEA (113 μl, 0.64 mmol) and N,N,N′,N′-Tetramethyl-O—(N-succinimidyl)uroniumtetrafluorborate (146 mg, 0.48 mmol) were added to the solution and the reaction was stirred for 2 h. Subsequently, the solvent was removed in vacuo and 12 (95 mg, 0.30 mmol, 92%) was isolated as a yellow solid, which was used without further purification.

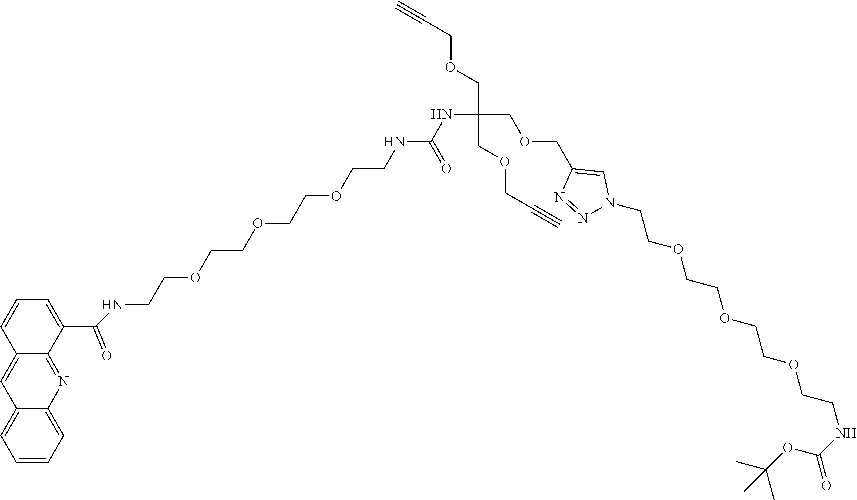 [SMILES: C#CCOCC(COCC#C)(COCc1cn(CCOCCOCCOCCNC(=O)OC(C)(C)C)nn1)NC(=O)NCCOCCOCCOCCNC(=O)c1cccc2cc3ccccc3nc12]

### Tert-Butyl (2-(2-(2-(2-(4-(20-(acridin-4-yl)-6,20-dioxo-4,4-bis((prop-2-yn-1-yloxy)methyl)-2,10,13,16-tetraoxa-5,7,19-triazaicosyl)-1H-1,2,3-triazol-1-yl)ethoxy)ethoxy)ethoxy)ethyl)carbamate 13

11 (75 mg, 0.10 mmol) and 12 (62 mg, 0.19 mmol) were dissolved in CH₂Cl₂ (1 ml) and 12 (32 mg, 0.10 mmol) in dichloromethane (1 mL) was added subsequently. The reaction was allowed to stir for 18 h at 40° C. The reaction was stopped by adding a saturated NaHCO₃ solution. The aqueous phase was extracted with CH₂Cl₂. The combined organic phases were dried over Na₂SO₄, filtered and the solvent was removed in vacuo. The crude product was purified by column chromatography (CH₃OH in CH₂Cl₂: 2%-10%). 13 (62 mg, 0.06 mmol, 65%) was isolated as a yellow foam.

¹H NMR (400 MHz, CDCl₃): δ=12.15 (s, 1H), 9.00 (d, J=7.1 Hz, 1H), 8.94 (s, 1H), 8.27 (d, J=8.8 Hz, 1H), 8.18 (d, J=8.3 Hz, 1H), 8.08 (d, J=8.4 Hz, 1H), 7.89 (t, J=7.8 Hz, 1H), 7.77 (s, 1H), 7.69 (t, J=7.8 Hz, 1H), 7.64 (t, J=7.6 Hz, 1H), 5.58 (s, 1H), 5.13 (s, 1H), 4.66 (s, 2H), 4.53 (t, J=5.1 Hz, 2H), 4.13 (s, 4H), 3.91 (t, J=6.0 Hz, 4.2, 3H), 3.87 (t, J=5.1 Hz, 3H), 3.84 (d, J=4.3 Hz, 3H), 3.82-3.80 (m, 2H), 3.79 (s, 2H), 3.75-3.71 (m, 2H), 3.60 (s, 10H), 3.54 (t, J=5.2 Hz, 2H), 3.51-3.47 (m, 2H), 3.44 (t, J=5.1 Hz, 2H), 3.32 (q, J=5.2 Hz, 2H), 3.26 (t, J=5.1 Hz, 2H), 2.70 (s, 1H), 2.44 (t, J=2.4 Hz, 1H), 1.44 (s, 9H).

¹³C NMR (101 MHz, CDCl₃): δ=172.1, 166.2, 157.9, 156.0, 132.5, 128.2, 126.8, 126.5, 126.0, 125.6, 123.8, 79.8, 74.6, 70.6, 70.6, 70.5, 70.5, 70.5, 70.4, 70.4, 70.2, 70.2, 70.2, 69.9, 69.5, 69.5, 64.9, 58.6, 50.2, 39.9, 39.8, 28.4, 25.4.

HRMS (ESI): m/z calculated for C₄₉H₆₉N₈O₁₃[M+H]⁺: 977.4984, found: 977.7980

R_{f} (15% CH₃OH in CH₂Cl₂): 0.15

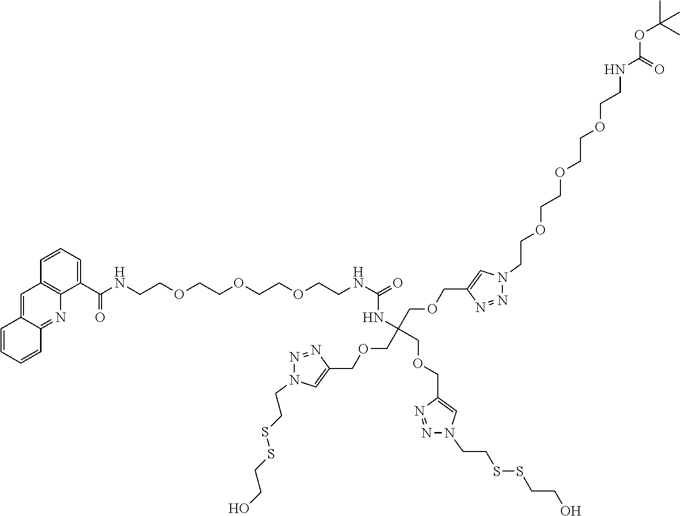 [SMILES: CC(C)(C)OC(=O)NCCOCCOCCOCCn1cc(COCC(COCc2cn(CCSSCCO)nn2)(COCc2cn(CCSSCCO)nn2)NC(=O)NCCOCCOCCOCCNC(=O)c2cccc3cc4ccccc4nc23)nn1]

### Tert-Butyl (2-(2-(2-(2-(4-(20-(acridin-4-yl)-4,4-bis(((1-(2-((2-hydroxyethyl)disulfaneyl)ethyl)-1H-1,2,3-triazol-4-yl)methoxy)methyl)-6,20-dioxo-2,10,13,16-tetraoxa-5,7,19-triazaicosyl)-1H-1,2,3-triazol-1-yl)ethoxy)ethoxy)ethoxy)ethyl)carbamate 14

13 (62 mg, 0.06 mmol), 57 (24 mg, 0.13 mmol), THPTA (3 mg, 10 mol %) and CuSO₄*5H₂0 (2 mg, 10 mol %) were dissolved in THF/H₂O (2/1, 650 μl) at room temperature. Sodium ascorbate (3 mg, 20 mol %) was added to the solution and was allowed to stir for 2 h. The solvents were removed under reduced pressure and the crude product was purified by column chromatography (CH₃OH in CH₂Cl₂: 0%-3%-10%). 14 (66 mg, 0.06 mmol, 87%) was isolated as a yellow oil.

1H NMR (400 MHz, CDCl₃): δ=12.12 (s, 1H), 8.98 (d, J=7.4 Hz, 2H), 8.26 (d, J=8.3 Hz, 1H), 8.19 (d, J=8.3 Hz, 1H), 8.09 (d, J=8.4 Hz, 1H), 7.90 (t, J=7.7 Hz, 1H), 7.77 (s, 3H), 7.67 (dt, J=15.4, 7.5 Hz, 2H), 5.80 (s, 1H), 5.46 (s, 1H), 5.16 (s, 1H), 4.68 (t, J=6.8 Hz, 4H), 4.60 (d, J=6.8 Hz, 6H), 4.53 (t, J=5.1 Hz, 2H), 3.89 (t, J=5.2 Hz, 6H), 3.83 (t, J=5.9 Hz, 6H), 3.81-3.78 (m, 2H), 3.78-3.74 (m, 6H), 3.74-3.69 (m, 3H), 3.60 (d, J=4.6 Hz, 11H), 3.53 (t, J=5.2 Hz, 2H), 3.49-3.46 (m, 2H), 3.42 (t, J=5.1 Hz, 2H), 3.30 (q, J=5.4 Hz, 2H), 3.25 (d, J=4.8 Hz, 2H), 3.19 (t, J=6.8 Hz, 4H), 2.87 (t, J=5.9 Hz, 4H), 1.44 (s, 9H).

HRMS (ESI): m/z calculated for C₅₇H₈₈N₁₄O₁₅S₄[M+2H]²⁺: 668.7757, found: 668.7752

R_{f} (10% CH₃OH in CH₂Cl₂): 0.3

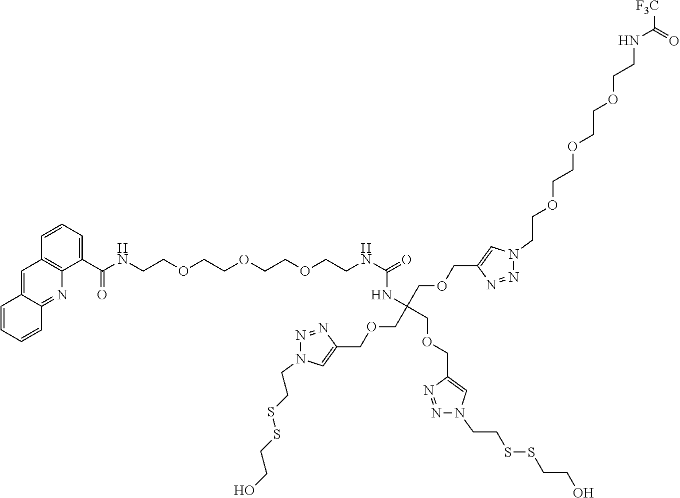 [SMILES: O=C(NCCOCCOCCOCCNC(=O)c1cccc2cc3ccccc3nc12)NC(COCc1cn(CCOCCOCCOCCNC(=O)C(F)(F)F)nn1)(COCc1cn(CCSSCCO)nn1)COCc1cn(CCSSCCO)nn1]

### N-(1-(1-(2-((2-hydroxyethyl)disulfaneyl)ethyl)-1H-1,2,3-triazol-4-yl)-4-(((1-(2-((2-hydroxyethyl)disulfaneyl)ethyl)-1H-1,2,3-triazol-4-yl)methoxy)methyl)-6-oxo-4-(((1-(1,1,1-trifluoro-2-oxo-6,9,12-trioxa-3-azatetradecan-14-yl)-1H-1,2,3-triazol-4-yl)methoxy)methyl)-2,10,13,16-tetraoxa-5,7-diazaoctadecan-18-yl)acridine-4-carboxamide

14 (52 mg, 0.04 mg) was dissolved in CH₂Cl₂ (400 μl) at room temperature. Trifluoroacetic acid (75 μl, 0.97 mmol) was added to the solution and the reaction was stirred for 15 h. The reaction was stopped by adding a saturated Na₂CO₃ solution. The reaction was stopped by adding a saturated Na₂CO₃ solution. The pH of the aqueous phase was adjusted to pH=11. The aqueous phase was extracted with CH₂Cl₂ and the combined organic phases were dried over Na₂SO₄, filtered and the solvent separated in vacuo. 15 (43 mg, 0.03 mmol, 83%) was isolated as a yellow oil.

¹H NMR (400 MHz, CDCl₃): δ=12.15 (s, 1H), 8.98 (d, J=8.1 Hz, 2H), 8.26 (d, J=8.8 Hz, 1H), 8.19 (d, J=8.3 Hz, 1H), 8.09 (d, J=8.5 Hz, 1H), 7.90 (t, J=7.8 Hz, 1H), 7.82 (s, 1H), 7.76 (d, J=4.6 Hz, 3H), 7.67 (dt, J=15.6, 7.6 Hz, 2H), 5.81 (s, 1H), 5.45 (s, 1H), 4.68 (t, J=6.8 Hz, 4H), 4.60 (d, J=7.3 Hz, 6H), 4.52 (t, J=5.1 Hz, 2H), 3.98-3.69 (broad set of signals with a total integral of 19H), 3.67-3.54 (m, 14H), 3.47 (t, J=5.8 Hz, 2H), 3.42 (t, J=5.0 Hz, 4H), 3.23 (t, J=4.8 Hz, 2H), 3.19 (t, J=6.8, 4H), 2.87 (t, J=5.9, 4H).

¹³C NMR (101 MHz, CDCl₃): δ=191.3, 158.1, 145.0, 144.7, 132.7, 128.3, 126.8, 126.6, 126.0, 124.0, 123.6, 70.6, 70.5, 70.5, 70.4, 70.4, 70.4, 70.2, 70.1, 69.9, 69.8, 69.3, 68.7, 64.8, 64.7, 59.9, 58.8, 53.5, 50.2, 49.0, 41.8, 39.8, 39.7, 37.9.

R_{f} (10% CH₃OH in CH₂Cl₂): 0.25

HRMS (ESI): m/z calculated for C₅₄H₇₉F₃N₁₄O₁₄S₄[M+2H]²⁺: 666.2368, found: 666.2361

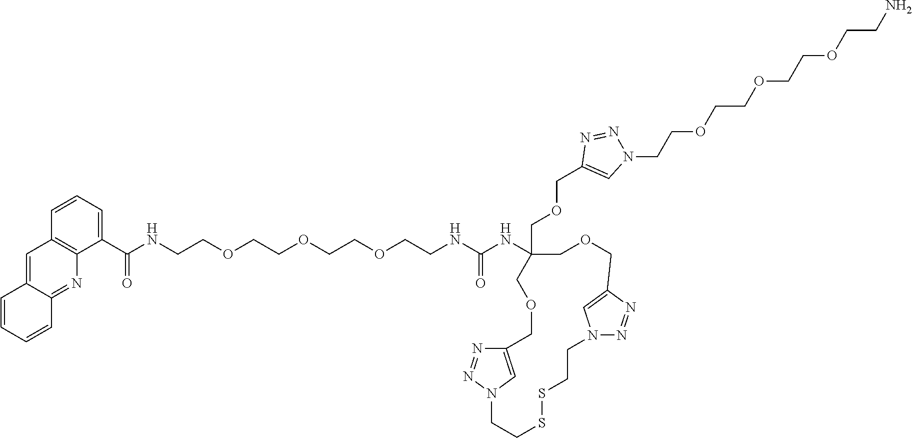 [SMILES: NCCOCCOCCOCCn1cc(COCC2(NC(=O)NCCOCCOCCOCCNC(=O)c3cccc4cc5ccccc5nc34)COCc3cn(nn3)CCSSCCn3cc(nn3)COC2)nn1]

### N-(1-(((1 ⁴Z,8⁴Z)-12-(((1-(2-(2-(2-(2-aminoethoxy)ethoxy)ethoxy)ethyl)-1H-1,2,3-triazol-4-yl)methoxy)methyl)-1 ¹H,8¹H-10,14-dioxa-4,5-dithia-1,8(1,4)-ditriazolacyclopentadecaphane-12-yl)amino)-1-oxo-5,8,11-trioxa-2-azatridecan-13-yl)acridine-4-carboxamide 16

15 (52 mg, 0.04 mmol) was dissolved in CH₃OH/1M NaOH (1/1, 400 μl) at room temperature and the reaction was allowed to stir for 1 h. The solvents were removed under reduced pressure and the crude product was filtered over silica (eluted with: CH₃OH in CH₂Cl₂: 20%). 16 (19 mg, 0.02 mmol, 45%) was isolated as a yellow oil.

R_{f} (20% CH₃OH in CH₂Cl₂): 0.5

HRMS (ESI): m/z calculated for C₄₈H₆₈N₁₄O₁₁S₂Na[M+Na]⁺: 1103.4531, found: 1103.4536

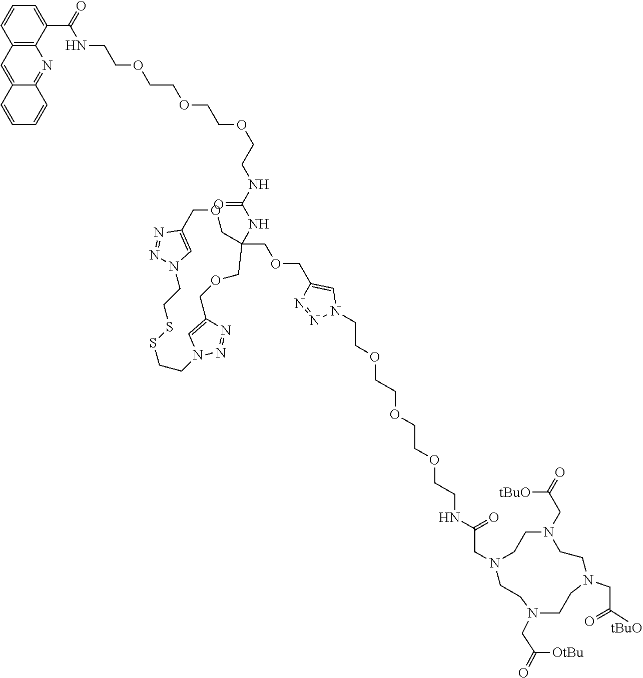 [SMILES: CC(C)(C)OC(=O)CN1CCN(CC(=O)NCCOCCOCCOCCn2cc(COCC3(NC(=O)NCCOCCOCCOCCNC(=O)c4cccc5cc6ccccc6nc45)COCc4cn(nn4)CCSSCCn4cc(nn4)COC3)nn2)CCN(CC(=O)OC(C)(C)C)CCN(CC(=O)OC(C)(C)C)CC1]

### Tri-tert-butyl 2,2′,2″-(10-14-(4-((((1⁴Z,8⁴Z)-12-(3-(1-(acridin-4-yl)-1-oxo-5,8,11-trioxa-2-azatridecan-13-yl)ureido)-1¹H,8¹H,10,14-dioxa-4,5-dithia-1,8(1,4)-ditriazolacyclopentadecaphane-12-yl)methoxy)methyl)-1H-1,2,3-triazol-1-yl)-2-oxo-6,9,12-trioxa-3-azatetradecyl)-1,4,7,10-tetraazacyclododecane-1,4,7-triyl)triacetate 17

DOTA (13 mg, 0.02 mmol) was dissolved in CH₂Cl₂/DMF (1/1, 1.8 ml) at room temperature. HATU (9 mg, 0.02 mmol, 1.3 eq.) and DIPEA (9 μl, 0.05 mmol, 3 eq.) were added to the solution and the solution was allowed to stir for 15 min. Then 16 (19 mg, 0.02 mmol, 1 eq.) was added to the solution and the reaction was allowed to stir for 15 h. The reaction mixture was then concentrated in vacuo. The crude product was purified by column chromatography (CH₃OH in CH₂Cl₂: 0%-10%-35%). 17 (22 mg, 0.01 mmol, 77%) was isolated as a yellow foam.

¹H NMR (500 MHz, CD₃OD): δ=8.52 (dd, J=4.3, 1.4 Hz, 2H), 8.43-8.29 (m, 1H), 8.18 (dd, J=8.4, 1.4 Hz, 1H), 8.02-7.91 (m, 3H), 7.84 (s, 1H), 7.33 (dd, J=8.4, 4.3 Hz, 2H), 4.86 (s, 1H), 4.61 (t, J=6.0 Hz, 3H), 4.59-4.46 (m, 7H), 3.89-3.87 (m, 3H), 3.86-3.81 (m, 2H), 3.80-3.72 (m, 4H), 3.72-3.66 (m, 4H), 3.64-3.51 (m, 14H), 3.48-3.38 (m, 7H), 3.22 (q, J=7.3 Hz, 9H), 2.96-1.87 (broad set of signals with a total integral of 18H), 1.57 (dd, J=7.9, 2.8, 5H), 1.53-1.45 (m, 18H), 1.37-1.30 (m, 16H).

¹³C NMR (125.8 MHz, CD₃OD): δ=173.0, 172.0, 147.2, 144.6, 138.4, 134.9, 134.8, 133.2, 131.7, 126.8, 119.5, 81.3, 70.3, 70.2, 70.2, 70.1, 70.1, 70.0, 69.9, 69.8, 69.5, 69.4, 69.3, 68.9, 68.8, 63.9, 63.6, 58.5, 50.0, 49.2, 46.5, 39.5, 39.4, 38.8, 27.1, 27.0, 7.8.

R_{f} (40% CH₃OH in CH₂Cl₂): 0.2

HRMS (ESI): m/z calculated for C₇₆H₁₂₀N₁₈O₁₈S₂[M+2H]²⁺: 818.4235, found: 818.4231

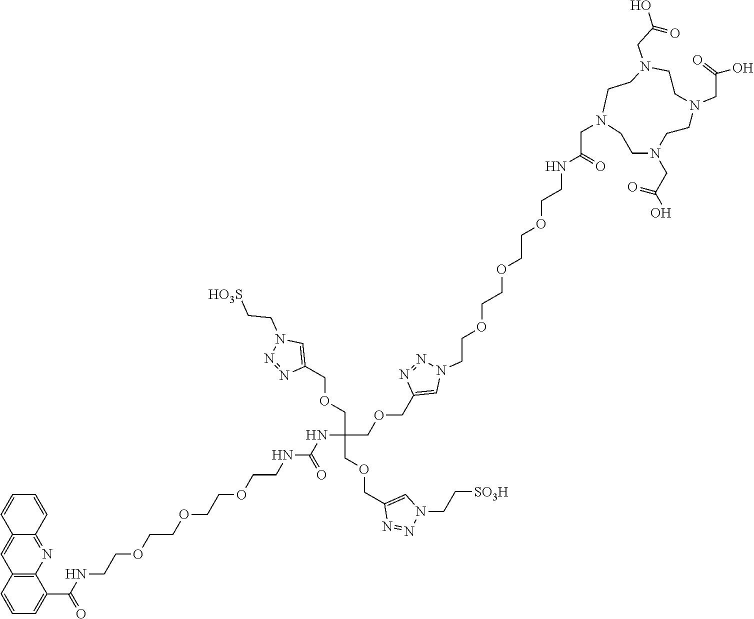 [SMILES: O=C(O)CN1CCN(CC(=O)O)CCN(CC(=O)NCCOCCOCCOCCn2cc(COCC(COCc3cn(CCS(=O)(=O)O)nn3)(COCc3cn(CCS(=O)(=O)O)nn3)NC(=O)NCCOCCOCCOCCNC(=O)c3cccc4cc5ccccc5nc34)nn2)CCN(CC(=O)O)CC1]

### 2,2′,2″-(10-(14-(4-(20-(acridin-4-yl)-6,20-dioxo-4,4-bis(((1-(2-sulfoethyl)-1H-1,2,3-triazol-4-yl)methoxy)methyl)-2,10,13,16-tetraoxa-5,7,19-triazalcosyl)-1H-1,2,3-triazol-1-yl)-2-oxo-6,9,12-trioxa-3-azatetradecyl)-1,4,7,10-tetraazacyclododecane-1,4,7-triyl)triacetic Acid 18

NCS (5 mg, 0.01 mmol, 50 mol %) was dissolved in CH₃CN/2M HCl (5.7/1, 300 μl) at 10° C. To the solution was added a solution of 17 (10 mg, 0.01 mmol) in CH₃CN/2M HCl (5.7/1, 300 μl) and the reaction was allowed to stir for 30 min at this temperature. After complete conversion to the disulfonic acid chloride, a 1M NaOH solution (2 ml) was added to the solution and stirring was continued for a further 2 h, allowing the reaction to warm to room temperature. The reaction mixture was concentrated in vacuo and the residue was taken up in CH₂Cl₂/TFA (1/1, 600 μl) and allowed to stir for another 18 h at room temperature. Subsequently, the solvents were removed in vacuo and the crude product was purified by column chromatography (C-18, gradient: 3 min 1% CH₃CN, 20 min gradual to 40% CH₃CN). 18 (6 mg, 0.6 μmol, 10%) was isolated after lyophilisation as a yellow solid.

HRMS (ESI): m/z calculated for C₇₆H₁₂₀N₁₈O₁₈S₂[M+2H]²⁺: 818.4235, found: 818.4231

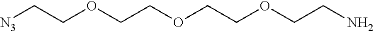 [SMILES: [N-]=[N+]=NCCOCCOCCOCCN]

### 2-(2-(2-(2-azidoethoxy)ethoxy)ethoxy)ethan-1-amine 19

Tetraethylene glycol (8.3 ml, 47.9 mmol) was dissolved in CH₂Cl₂ (48 ml) and cooled to 0° C. Methanesulfonyl chloride (8.2 ml, 105.3 mmol) and triethylamine (14.0 ml, 105.3 mmol) were added to the solution. The resulting suspension was stirred for 16 h and warmed up to room temperature. The solvent was then removed under reduced pressure and the residue was taken up in aqueous HCl solution (1 M). The aqueous solution was extracted with CH₂Cl₂ and the combined organic phases were washed with aqueous NaHCO₃ solution and dried over Na₂SO₄, filtered and the solvent was removed under reduced pressure. The crude product was dissolved in DMF (100 ml) and sodium azide (6.9 g, 105.3 mmol) was added to the solution. The reaction was stirred for 18 h at 60° C. The reaction solution was added to water and extracted with EtOAc. The organic phase was Na₂SO₄ dried, filtered and the solvent was removed under reduced pressure. 19 (11.1 g, 45.5 mmol, 95%) was obtained as a yellow oil.

¹H NMR (400 MHz, CDCl₃): δ=4.40 (s, 2H), 4.04-3.56 (m, 12H), 3.47-3.36 (m, 2H), 3.00 (t, J=5.1 Hz, 2H).

The spectroscopic data are in agreement with the literature.^{[3]}

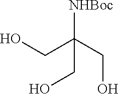 [SMILES: CC(C)(C)OC(=O)NC(CO)(CO)CO]

### Tert-Butyl (1,3-dihydroxy-2-(hydroxymethyl)propan-2-yl)carbamate 20

Tris (200 mg, 1.65 mmol) was dissolved in CH₃OH (6.7 ml). To the solution was added a solution of tert-butyl dicarbonate (0.4 ml, 1.72 mmol) in CH₃OH (1.5 ml). The reaction was allowed to stir overnight. The solvent was removed under reduced pressure and 20 (350 mg, 1.58 mmol, 96%) was obtained as a white solid.

¹H NMR (400 MHz, CD₃OD): δ 3.71 (s, 6H), 1.46 (s, 9H).

The spectroscopic data are in agreement with the literature.^{[4]}

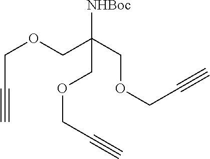 [SMILES: C#CCOCC(COCC#C)(COCC#C)NC(=O)OC(C)(C)C]

### Tert-Butyl (1,3-bis(prop-2-yn-1-yloxy)-2-((prop-2-yn-1-yloxy)methyl)propan-2-yl)carbamate 21

20 (930 mg, 4.2 mmol) was dissolved in DMF (11.3 ml) and cooled to 0° C. Propargyl bromide (80% w/w in toluene 2.8 ml, 25.2 mmol) was added to the solution and over a period of 20 minutes, powdered KOH (1.4 g, 25.2 mmol) was added to the reaction in portions and the reaction was stirred for 4 h at 37° C. EtOAc was then added to the reaction and the solution was washed with water. The organic phase was dried over Na₂SO₄, filtered and the solvent was removed under reduced pressure. The crude product was purified by column chromatography (PE/EE=9/1). 21 (1.04 g, 3.11 mmol, 74%) was isolated as a yellow oil.

¹H NMR (400 MHz, CDCl₃): δ=4.17 (d, J=2.4 Hz, 1H), 3.80 (s, 1H), 2.44 (t, J=2.4 Hz, OH), 1.44 (s, 1H).

The spectroscopic data are in agreement with the literature.^{[5]}

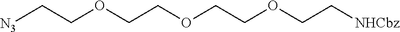 [SMILES: [N-]=[N+]=NCCOCCOCCOCCNC(=O)OCc1ccccc1]

### Benzyl (2-(2-(2-(2-azidoethoxy)ethoxy)ethoxy)ethyl)carbamate 22

19 (1.70 g, 7.79 mmol) was dissolved in 1,4-dioxane/H₂O (1/1, 18 ml) at room temperature and NaHCO₃ (1.96 g, 23.38 mmol) was added to the solution. To this suspension, chloroformic acid benzyl ester (2.0 ml, 9.35 mmol) in 1,4-dioxane/H₂O (1/1, 18 ml) was added dropwise and the resulting suspension was stirred for 18 h. The reaction was stopped by adding water. The reaction was stopped by addition of water and the aqueous phase was extracted with CH₂Cl₂. The combined organic phases were dried over Na₂SO₄, filtered and the solvent was removed in vacuo. The crude product was purified by silica gel chromatographed (PE/EE=1/1.22 (2.37 g, 6.72 mmol, 86%) was obtained as a colourless oil.

¹H NMR (400 MHz, CDCl₃): δ=7.31-7.48 (m, 5H), 5.35 (s, 1H), 5.12 (s, 2H), 3.66 (d, J=7.6 Hz, 10H), 3.59 (t, J=5.1 Hz, 2H), 3.48-3.33 (m, 4H).

¹³C NMR (101 MHz, CDCl₃): δ=156.5, 136.6, 128.5, 128.1, 128.19, 70.7, 70.7, 70.6, 70.3, 70.0, 70.2, 66.7, 50.7, 40.9.

R_{f} (PE/EE=1/2): 0.45

HRMS (ESI): m/z calculated for C₁₆H₂₅N₄O₅[M+H]⁺: 353.1825, found: 353.1831

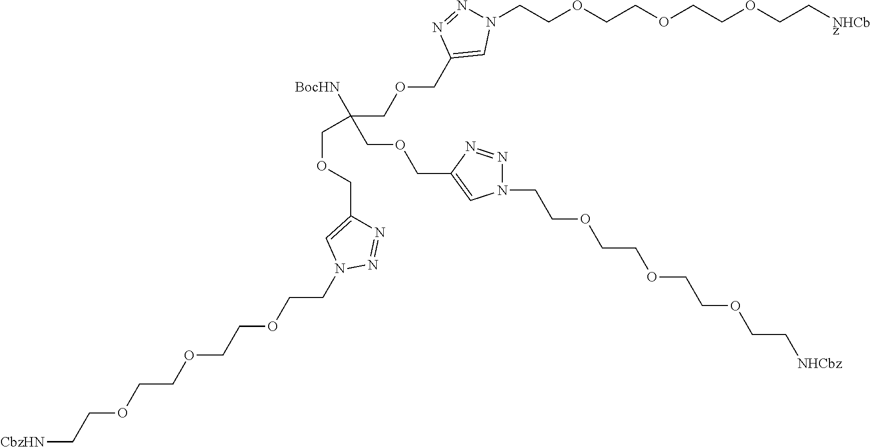 [SMILES: CC(C)(C)OC(=O)NC(COCc1cn(CCOCCOCCOCCNC(=O)OCc2ccccc2)nn1)(COCc1cn(CCOCCOCCOCCNC(=O)OCc2ccccc2)nn1)COCc1cn(CCOCCOCCOCCNC(=O)OCc2ccccc2)nn1]

### TERT-BUTYL (1,3-BIS((1-(3-OXO-1-PHENYL-2,7,10,13-TETRAOXA-4-AZAPENTADECAN-15-YL)-1H-1,2,3-triazol-4-yl)methoxy)-2-(((1-(3-oxo-1-phenyl-2,7,10,13-tetraoxa-4-azapentadecan-15-yl)-1H-1,2,3-triazol-4-yl)methoxy)methyl)propan-2-yl)carbamate 23

22 (1004 mg, 3.0 mmol) and 21 (1055 mg, 3.0 mmol) were placed in THF (30 ml) at room temperature. To this solution were added solutions of CuSO₄·5 H₂O (97 mg, 0.4 mmol) and THPTA (520 mg, 1.2 mmol) in H₂O (3 mL) and sodium ascorbate (237 mg, 1.2 mmol) in H₂O (3 mL). The reaction mixture was stirred for 18 h and then diluted with water and EtOAc. The aqueous phase was extracted with EtOAc. The combined organic phases were dried over Na₂SO₄, filtered and the solvent was removed in vacuo. The crude product was purified by column chromatography (PE/EE=1/9). 23 (3635 mg, 2.61 mmol, 87%) was isolated as a yellow oil.

¹H NMR (400 MHz, CDCl₃): δ=7.66 (s, 3H), 7.36-7.27 (m, 15H), 5.46 (d, J=6.2 Hz, 3H), 5.07 (s, 2H), 5.02 (s, 1H), 4.56 (s, 2H), 4.45 (t, J=5.2 Hz, 6H), 3.80 (t, J=5.2 Hz, 6H), 3.72 (s, 6H), 3.60-3.51 (m, 30H), 3.36 (q, J=5.4 Hz, 6H), 1.37 (s, 9H).

¹³C NMR (101 MHz, CDCl₃): δ=156.5, 154.8, 144.6, 136.6, 128.5, 128.2, 128.1, 123.8, 70.5, 70.5, 70.20, 70.0, 69.4, 69.3, 66.6, 64.8, 58.5, 53.5, 50.1, 40.8, 28.5.

R_{f} (8% CH₃OH in CH₂Cl₂): 0.25

HRMS (ESI): m/z calculated for C₆₆H₉₇N₁₃O₂₀[M+H]⁺: 1392.7051, found: 1392.7049

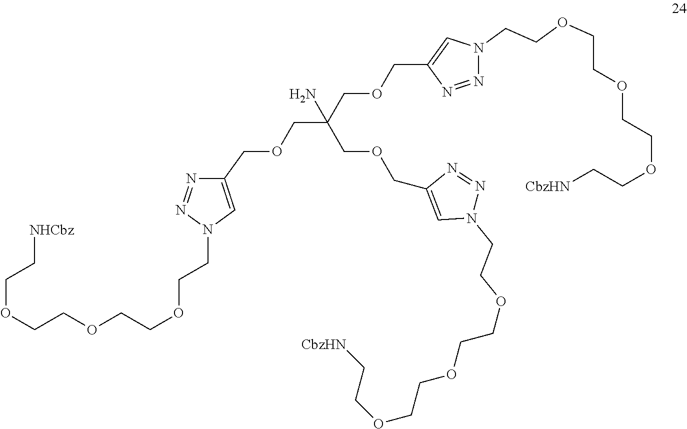 [SMILES: NC(COCc1cn(CCOCCOCCOCCNC(=O)OCc2ccccc2)nn1)(COCc1cn(CCOCCOCCOCCNC(=O)OCc2ccccc2)nn1)COCc1cn(CCOCCOCCOCCNC(=O)OCc2ccccc2)nn1]

23 (200 mg, 0.14 mmol) were dissolved in CH₂Cl₂ (1.5 ml) at room temperature and TFA (220 μl, 2.87 mmol) was added to the solution. The reaction was stirred for 6 h and stopped by adding a saturated Na₂CO₃ solution. The aqueous phase was extracted with CH₂Cl₂ and the combined organic phases were dried over Na₂SO₄, filtered and the solvent was removed in vacuo. The resulting amine 24 (166 mg, 0.13 mmol, 89%) was obtained as a yellow oil and used without further purification.

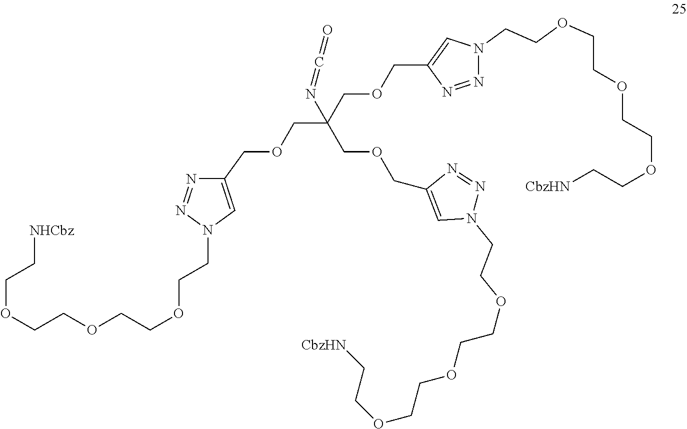 [SMILES: O=C=NC(COCc1cn(CCOCCOCCOCCNC(=O)OCc2ccccc2)nn1)(COCc1cn(CCOCCOCCOCCNC(=O)OCc2ccccc2)nn1)COCc1cn(CCOCCOCCOCCNC(=O)OCc2ccccc2)nn1]

Triphosgene (38 mg, 0.13 mmol) was dissolved in CH₂Cl₂ (0.4 ml). To this solution were added solutions of triethylamine (40 μl, 0.30 mmol) in CH₂Cl₂ (0.4 ml) and 24 (166 mg, 0.13 mmol) in CH₂Cl₂ (0.4 ml), the mixture was allowed to stir for 1 h. Saturated NaHCO₃ solution was added to the reaction solution, the aqueous phase was extracted with CH₂Cl₂ and the combined organic phases were dried over Na₂SO₄, filtered and the solvent was removed in vacuo. Isocyanate 25 (160 mg, 0.12 mmol, 95%) was obtained as a yellow oil and further used without further purification steps.

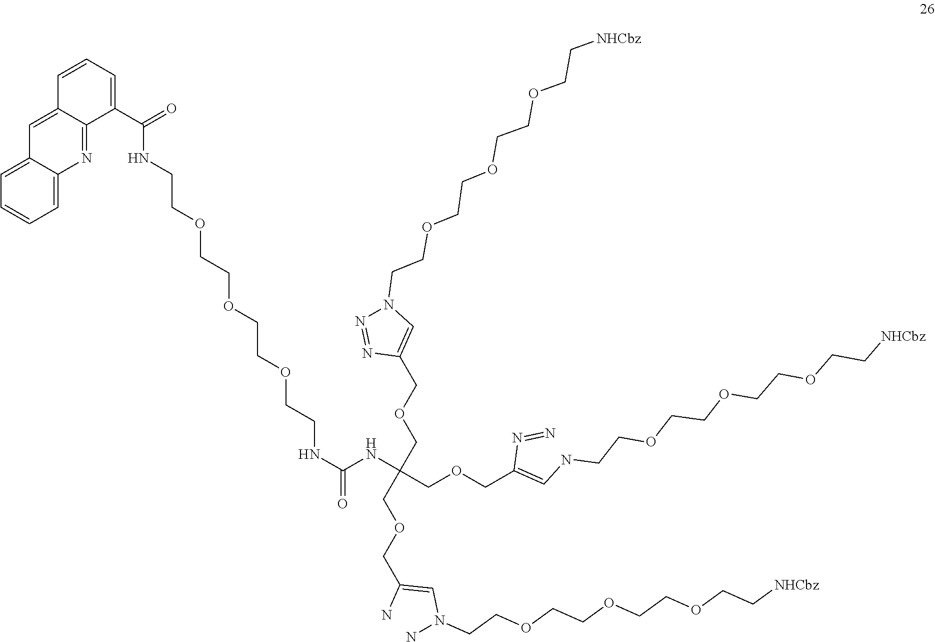 [SMILES: N=N/C(=C\NCCOCCOCCOCCNC(=O)OCc1ccccc1)COCC(COC/C(N)=C/N(N)CCOCCOCCOCCNC(=O)OCc1ccccc1)(COCc1cn(CCOCCOCCOCCNC(=O)OCc2ccccc2)nn1)NC(=O)NCCOCCOCCOCCNC(=O)c1cccc2cc3ccccc3nc12]

25 (160 mg, 0.12 mmol) and 33 (53 mg, 0.13 mmol) were dissolved in CH₂Cl₂ (1.2 ml) at room temperature. Triethylamine (25 μl, 0.18 mmol) was added to the solution and the solution was allowed to stir for 2 h. Water was added to the reaction and the aqueous phase was extracted with CH₂Cl₂. The combined organic phases were dried over Na₂SO₄, filtered and the solvent was removed under reduced pressure. The crude product was purified by column chromatography (5% CH₃OH in CH₂Cl₂). 26 (158 mg, 0.09 mmol, 76%) was isolated as a yellow oil.

¹H NMR (400 MHz, CDCl₃): δ=8.99 (d, J=7.1 Hz, 1H), 8.93 (s, 1H), 8.26 (d, J=8.7 Hz, 1H), 8.17 (d, J=8.3 Hz, 1H), 8.07 (d, J=8.6 Hz, 1H), 7.88 (t, J=7.8 Hz, 1H), 7.74-7.56 (m, 5H), 7.42-7.29 (m, 15H), 5.56 (s, 3H), 5.39 (s, 1H), 5.05-5.18 (bs, 6H), 4.53-4.66 (bs, 6H), 4.37-4.52 bs, 6H), 3.88-4.02 (m, 4H), 3.75-3.83 (bs, 12H), 3.66-3.72 (bs, 2H), 3.49-3.64 (bs, 32H), 3.46 (s, 2H), 3.37 (s, 8H), 3.23 (t, J=5.2 Hz, 2H).

¹³C NMR (101 MHz, CDCl₃): δ=166.1, 157.9, 156.5, 144.7, 144.5, 141.2, 136.6, 132.5, 128.5, 128.1, 128.1, 126.8, 126.5, 126.0, 125.5, 123.8, 70.6, 70.4, 70.2, 70.1, 70.0, 69.9, 69.4, 66.6, 64.8, 58.9, 53.5, 50.1, 40.8, 39.7, 31.9, 29.7, 29.7, 29.4, 22.7, 14.1.

R_{f} (10% CH₃OH in CH₂Cl₂): 0.7

HRMS (ESI): m/z calculated for C₈₄H₁₁₆N₁₆O₂₃[M+2H]²⁺: 858.4200, found: 858.3991

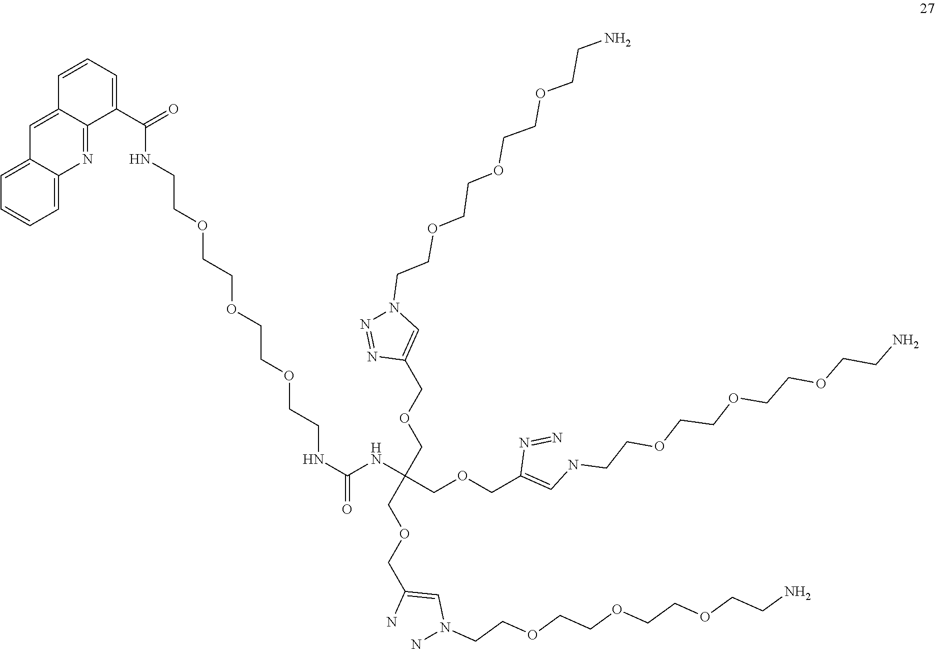 [SMILES: N=N/C(=C\NCCOCCOCCOCCN)COCC(COC/C(N)=C/N(N)CCOCCOCCOCCN)(COCc1cn(CCOCCOCCOCCN)nn1)NC(=O)NCCOCCOCCOCCNC(=O)c1cccc2cc3ccccc3nc12]

26 (26 mg, 0.02 mmol) was dissolved at room temperature in a solution of 33% (w/w) HBr in AcOH/AcOH (1/1, 150 μl) and stirred for 1.5 h. Diethyl ether was added to the reaction solution. The formed precipitate was filtered, washed with diethyl ether and dried in vacuo. Amine 27 (27 mg, quant.) was isolated as trihydrodromide and used without further purification step.

HRMS (ESI): m/z calculated for C₆₀H₉₆N₁₆O₁₇Na[M+Na]⁺: 1335.7037, found: 1335.7028.

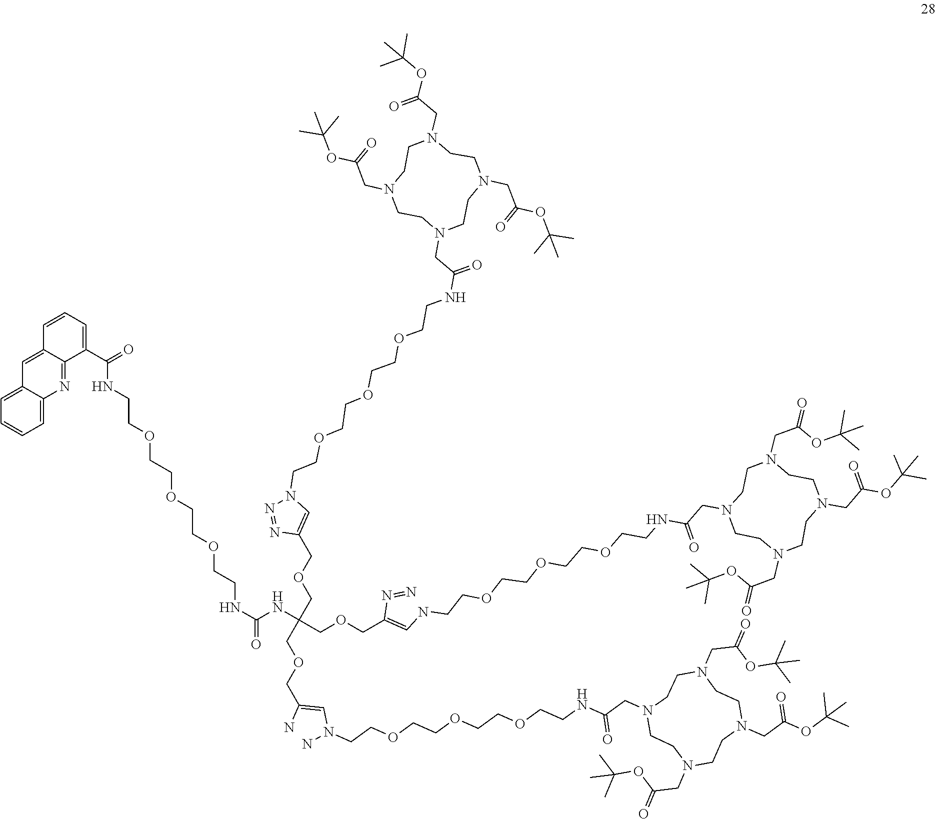 [SMILES: CC(C)(C)OC(=O)CN1CCN(CC(=O)NCCOCCOCCOCCN/C=C(/COCC(COC/C(N)=C/N(N)CCOCCOCCOCCNC(=O)CN2CCN(CC(=O)OC(C)(C)C)CCN(CC(=O)OC(C)(C)C)CCN(CC(=O)OC(C)(C)C)CC2)(COCc2cn(CCOCCOCCOCCNC(=O)CN3CCN(CC(=O)OC(C)(C)C)CCN(CC(=O)OC(C)(C)C)CCN(CC(=O)OC(C)(C)C)CC3)nn2)NC(=O)NCCOCCOCCOCCNC(=O)c2cccc3cc4ccccc4nc23)N=N)CCN(CC(=O)OC(C)(C)C)CCN(CC(=O)OC(C)(C)C)CC1]

To a solution of 36 (132 mg, 0.2 mmol) in DMF (1 ml), HATU (85 mg, 0.22 mmol) and DIPEA (90 μl) were added and subsequently the mixture was allowed to stir for 15 min at room temperature. To this solution 27 (77 mg, 0.05 mmol) in DMF (500 μl) was added and the reaction was stirred for 24 h. The solvent was then removed under reduced pressure. The residue was taken up in CH₂Cl₂ and the organic phase was washed with water. The combined aqueous phases were back-extracted with CH₂Cl₂/iPrOH (3/1). The combined organic phases were dried over Na₂SO₄, filtered and the volatiles were removed under reduced pressure. The crude product was purified by column chromatography (CH₃OH in CH₂Cl₂ 10%-15%-20%). 28 (46 mg, 0.02 mmol, 31%) was isolated as a yellow solid.

¹H NMR (400 MHz, CD₃OD): δ=9.19 (s, 1H), 8.84 (dd, J=7.1, 1.6 Hz, 1H), 8.37-8.33 (m, 1H), 8.30 (d, J=8.8, 1.0, 1H), 8.19 (d, J=8.4, 1H), 8.04-7.96 (m, 4H), 7.80-7.63 (m, 2H), 4.51-4.60 (m, 14H), 3.85-3.93 (m, 10H), 3.64-3.82 (bs, 15H), 3.48-3.62 (bs, 40H), 1.81-3.16 (bs, 72H), 1.48 (bs, 81H).

¹³C NMR (101 MHz, CD₃OD): δ 173.0, 172.7, 172.0, 147.6, 144.4, 133.3, 131.8, 128.6, 128.3, 127.4, 127.0, 126.5, 126.2, 124.3, 81.4, 81.3, 70.3, 70.2, 70.1, 70.1, 70.0, 69.9, 69.6, 69.3, 69.0, 64.0, 58.7, 55.9, 55.4, 55.3, 39.5, 38.8, 27.1, 27.0.

R_{f} (20% CH₃OH in CH₂Cl₂): 0.35

HRMS (ESI): m/z calculated for C₁₁₄H₂₄₉N₂₈O₃₈[M+3H]³⁺: 872.9471, found: 872.9466

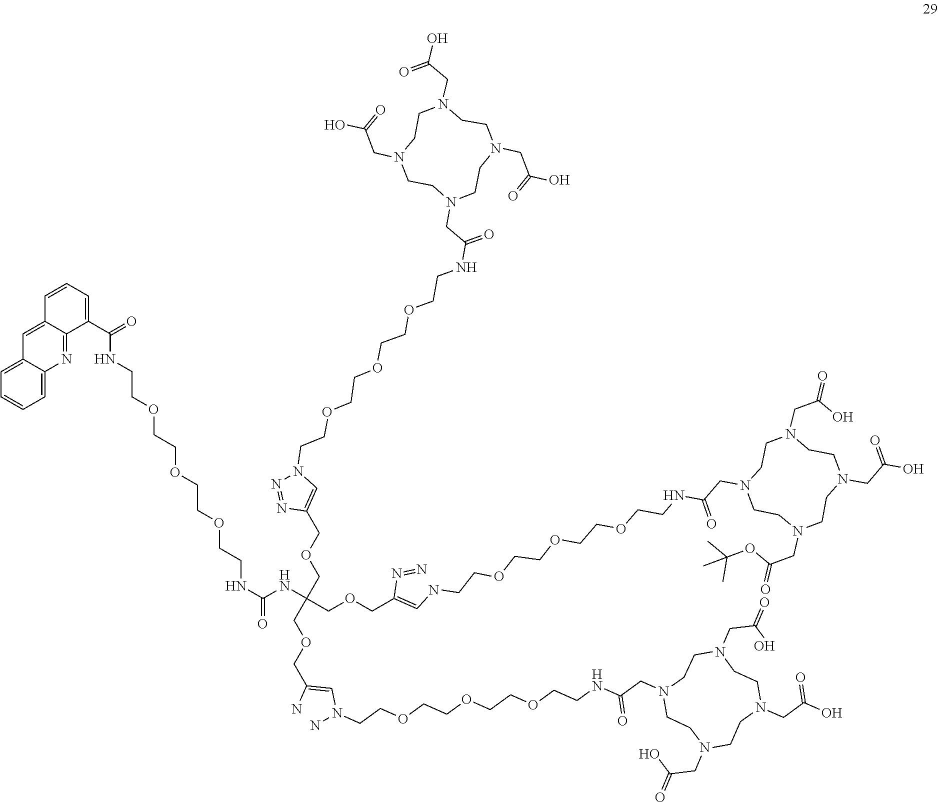 [SMILES: CC(C)(C)OC(=O)CN1CCN(CC(=O)O)CCN(CC(=O)O)CCN(CC(=O)NCCOCCOCCOCCN/C=C(/COCC(COC/C(N)=C/N(N)CCOCCOCCOCCNC(=O)CN2CCN(CC(=O)O)CCN(CC(=O)O)CCN(CC(=O)O)CC2)(COCc2cn(CCOCCOCCOCCNC(=O)CN3CCN(CC(=O)O)CCN(CC(=O)O)CCN(CC(=O)O)CC3)nn2)NC(=O)NCCOCCOCCOCCNC(=O)c2cccc3cc4ccccc4nc23)N=N)CC1]

28 (41 mg, 0.01 mmol) was suspended in CH₂Cl₂/TFA (5/1, 400 μl) at room temperature and stirred for 48 h. The reaction solution was then mixed with Et₂O and the resulting precipitate was filtered and washed several times with Et₂O. The crude product was then coevaporated several times with CH₂Cl₂. The residue was taken up in water and freeze-dried. 29 (40 mg, quant.) was obtained as an orange solid.

¹H NMR (400 MHz, D₂O): δ=9.66 (s, 1H), 8.52 (d, J=7.3 Hz, 1H), 8.44 (d, J=8.4 Hz, 1H), 8.28 (d, J=8.5 Hz, 1H), 8.19 (d, J=3.2 Hz, 2H), 7.89 (s, 3H), 7.78-7.85 (m, 3H), 4.35-4.545 (m, 20H), 2.87-4.14 (m, 220H) ppm.

HRMS (ESI): m/z calculated for C₁₀₈H₁₇₄N₂₈O₃₈[M+3H]³⁺: 824.7593, found: 824.7601

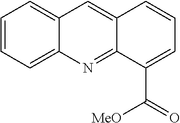 [SMILES: COC(=O)c1cccc2cc3ccccc3nc12]

### Methyl Acridine-4-carboxylate

2-Bromobenzaldehyde (1652 mg, 8.9 mmol), bis(dibenzylideneacetone)palladium (227 mg, 0.3 mmol), xantphos (287 mg, 0.5 mmol) and Cs₂CO₃ (2742 mg, 19.9 mmol) were suspended in 1,4-dioxane (degassed, 20 ml) at room temperature. The resulting suspension was stirred for 5 minutes and methyl anthranilate (1500 mg, 9.9 mmol) was added to the mixture. Subsequently, the reaction was stirred for 18 h at 115° C. After this time, the reaction solution was concentrated in vacuo and the residue was partitioned between saturated NH₄Cl solution and EtOAc. The aqueous phase was extracted with EtOAc and the combined organic phases were dried over Na₂SO₄, filtered and the solvent was removed in vacuo.

The crude product obtained was taken up in 5 ml of concentrated sulphuric acid and stirred for 1 h at 80° C. The resulting solution was poured into cold water and the formed precipitate was filtered. The filtrate was adjusted to pH=11 by addition of NaOH and the aqueous phase was extracted with EtOAc. The combined organic phases were dried over Na₂SO₄, filtered and the solvent was removed in vacuo. The crude product was purified by column chromatography (PE/EE=15/1-6/1). 30 (1368 mg, 5.8 mmol, 65%) was obtained as a yellow oil.

¹H NMR (400 MHz, CDCl₃): δ 8.75 (s, 1H), 8.29 (d, J=8.9 Hz, 1H), 8.06-8.14 (m, 2H), 7.95 (d, J=8.9 Hz, 1H), 7.73-7.82 (m, 1H), 7.47-7.57 (m, 2H), 4.11 (s, 3H) ppm.

The spectroscopic data are in agreement with the literature.^{[6]}

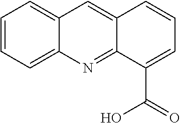 [SMILES: O=C(O)c1cccc2cc3ccccc3nc12]

### Acridine-4-carboxylic Acid 31

30 (1328 mg, 5.6 mmol) was dissolved in THF (56 mL) at room temperature. A LiOH solution (1 M, 9.0 mmol, 9.0 mL) was added to the solution and stirred for 12 h. The reaction solution was then concentrated in vacuo. After this time, the reaction solution was concentrated in vacuo. The residue was adjusted to pH=2 with a KHSO₄ solution. The aqueous phase was extracted with EtOAc. The combined organic phases were dried over Na₂SO₄, filtered and the solvent was removed in vacuo. 31 (735 mg, 3.3 mmol) was obtained as a yellow oil and used in the next step without further purification steps.

¹H NMR (400 MHz, CDCl₃): δ=9.04 (s, 1H), 8.93 (dd, J=7.1, 1.5 Hz, 1H), 8.29 (dd, J=8.4, 1.5 Hz, 1H), 8.25 (dd, J=8.8, 1.0 Hz, 1H), 8.13 (d, J=9.1 Hz, 1H), 7.99-7.91 (m, 1H), 7.81-7.63 (m, 2H).

The spectroscopic data are in agreement with the literature.^{[7]}

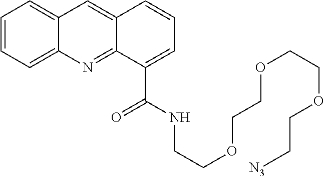 [SMILES: [N-]=[N+]=NCCOCCOCCOCCNC(=O)c1cccc2cc3ccccc3nc12]

### N-(2-(2-(2-(2-azidoethoxy)ethoxy)ethoxy)ethyl)acridine-4-carboxamide 32

31 (735 mg, 3.3 mmol) were dissolved at −15° C. in CH₂Cl₂/DMF (1/1, 33 ml). To the solution were added EDC-HCl (1388 mg, 7.2 mmol), oxyma (1029 mg, 7.2 mmol) and NaHCO₃ (1383 mg, 16.5 mml). The solution was stirred for 5 min and 19 (790 mg, 3.6 mmol) was added to the reaction. The mixture was stirred for 18 h, during which time it warmed to room temperature. The reaction was stopped after this time by the addition of water. The aqueous phase was extracted with EtOAc and the combined organic phases were washed with saturated NH₄Cl solution, saturated NaHCO₃ solution, water and saturated NaCl solution. The organic phase was dried over Na₂SO₄, filtered and the solvent was removed in vacuo. The crude product was purified by column chromatography (PE/EE=1/2-1/8). 32 (873 mg, 2.1 mmol, 38% o2s) was obtained as a yellow oil.

¹H NMR (400 MHz, CDCl₃): δ=12.11 (s, 1H), 8.99 (d, J=7.0 Hz, 1H), 8.89 (s, 1H), 8.25 (d, J=8.8 Hz, 1H), 8.14 (d, J=8.4 Hz, 1H), 8.04 (d, J=8.4 Hz, 1H), 7.82-7.89 (m, 1H), 7.57-7.71 (m, 2H), 3.83-3.94 (m, 4H), 3.73-3.82 (m, 4H), 3.64-3.69 (m, 2H), 3.53-3.60 (m, 3H), 3.28-3.35 (m, 2H) ppm.

¹³C NMR (101 MHz, CDCl₃): δ=166.0, 135.4, 132.4, 131.4, 129.1, 128.1, 126.8, 126.4, 126.0, 125.5, 121.3, 119.6, 117.7, 70.8, 70.7, 70.7, 70.5, 70.3, 70.0, 50.6, 39.7, 29.7 ppm.

R_{f} (PE/EE=1/5): 0.35

HRMS (ESI): m/z calculated for C₂₂H₂₅N₄O₄Na[M+Na]⁺: 432.1774, found: 432.1776

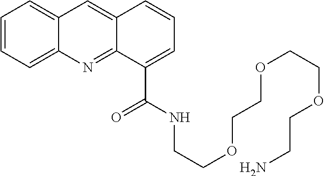 [SMILES: NCCOCCOCCOCCNC(=O)c1cccc2cc3ccccc3nc12]

### N-(2-(2-(2-(2-aminoethoxy)ethoxy)ethoxy)ethyl)acridine-4-carboxamide 33

32 (168 mg, 0.4 mmol) was dissolved in degassed THF/H2O (5/1, 4 ml) at room temperature. To the solution was added trimethylphosphine (1 M in THF, 0.5 mmol, 0.5 ml) and the solution was allowed to stir for 3 h. The solvent was then removed from the solution. The solvent was removed under reduced pressure and the residue was taken up in CH₂Cl₂ and extracted with HCl (1M). The combined aqueous phase was adjusted to pH=11 with saturated Na₂CO₃ solution and was subsequently extracted with CH₂Cl₂. The combined organic phases were dried over Na₂SO₄, filtered and the solvent was removed in vacuo. 33 (145 mg, 0.4 mmol, 92%) was obtained as a yellow oil and further used without further purification steps.

R_{f} (10% CH₃OH in CH₂Cl₂): 0.12

HRMS (ESI): m/z calculated for C₂₂H₂₇N₄O₄Na[M+Na]⁺: 406.1869, found: 406.1866.

¹H NMR (400 MHz, CDCl₃): δ 12.14 (s, 1H), 8.96 (dd, J=7.1, 1.6 Hz, 1H), 8.87 (s, 1H), 8.24 (d, J=8.8 Hz, 1H), 8.13 (dd, J=8.4, 1.6 Hz, 1H), 8.03 (d, J=8.4 Hz, 1H), 7.73-7.48 (m, 2H), 3.82-3.96 (m, 4H), 3.82-3.71 (m, 4H), 3.70-3.61 (m, 4H), 3.55 (m, 2H), 3.51-3.42 (m, 2H), 2.81 (s, 2H) ppm,

¹³C NMR (101 MHz, CDCl₃): δ 166.1, 147.5, 146.3, 137.7, 135.4, 132.4, 131.3, 130.1 6, 129.2, 128.1, 126.8, 126.4, 126.0, 125.5, 70.63, 70.6, 70.4, 70.36, 70.2, 69.9, 39.7, 29.7 ppm.

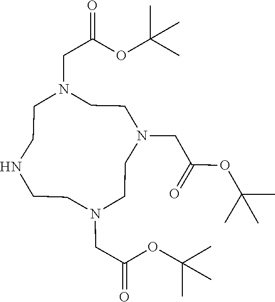 [SMILES: CC(C)(C)OC(=O)CN1CCNCCN(CC(=O)OC(C)(C)C)CCN(CC(=O)OC(C)(C)C)CC1]

### Tri-Tert-Butyl 2,2′,2″-(1,4,7,10-tetraazacyclododecane-1,4,7-triyl)triacetate 34

Cyclene (1000 mg, 5.80 mmol) was added to DMA (11.6 ml) and cooled to 0° C. NaOAc (1.43 g, 17.41 mmol) was added to the reaction and tert-butyl bromoacetate (2.6 ml, 17.41 mmol) was added dropwise. The solution was stirred for 72 h and warmed to room temperature. The product was precipitated from the solution by addition of aqueous NaHCO₃. After filtration and drying, trialkylated cyclene 34 (2.2 g, 4.32 mmol 73%) was obtained as a white solid.

¹H NMR (400 MHz, CDCl3): δ 10.02 (s, 1H), 3.36 (s, 4H), 3.28 (s, 2H), 3.09 (s, 4H), 2.89 (dd, J=20.5, 5.8 Hz, 12H), 1.44 (s, 27H).

The spectroscopic data are in agreement with the literature.^{[14]}

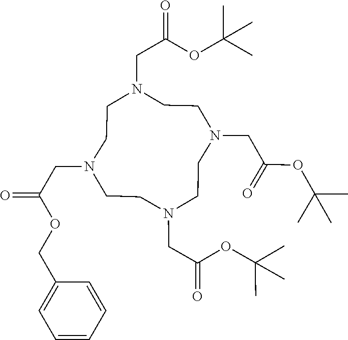 [SMILES: CC(C)(C)OC(=O)CN1CCN(CC(=O)OCc2ccccc2)CCN(CC(=O)OC(C)(C)C)CCN(CC(=O)OC(C)(C)C)CC1]

### Tri-Tert-Butyl 2,2′,2″-(10-(2-(benzyloxy)-2-oxoethyl)-1,4,7,10-tetraazacyclododecane-1,4,7-triyl)triacetate 35

K₂CO₃ (1.1 g, 8.23 mmol) was placed in DMA (100 ml) and 34 (2.1 g, 4.27 mmol) was added to the suspension. Benzyl bromoacetate (684 μl, 4.32 mmol) in CH₃CN (10 ml) was added to the reaction and stirred at 70° C. for 18 h. The reaction mixture was filtered and the solvent was removed under reduced pressure. The crude product was purified by column chromatography (10% CH₃OH/CH₂Cl₂). 35 (2.7 g, quantitative) was obtained as a white solid.

¹H NMR (400 MHz, CDCl₃): δ 7.36 (dt, J=9.7, 5.1 Hz, 1H), 5.16 (d, J=19.7 Hz, OH), 3.93-2.00 (m, 2H), 1.47 (s, 1H).

The spectroscopic data are in agreement with the literature.^{[15]}

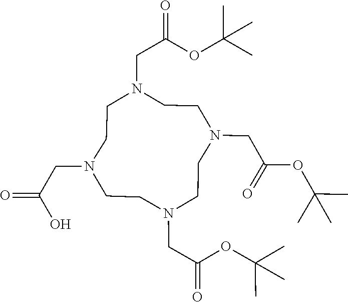 [SMILES: CC(C)(C)OC(=O)CN1CCN(CC(=O)O)CCN(CC(=O)OC(C)(C)C)CCN(CC(=O)OC(C)(C)C)CC1]

### 2-(4,7,10-tris(2-(tert-butoxy)-2-oxoethyl)-1,4,7,10-tetraazacyclododecan-1-yl)acetic Acid 36

35 (315 mg, 0.48 mmol) was dissolved in CH₃OH (12 mL) at room temperature. Palladium on activated carbon (10%, 10 mg) was added to the solution and a stream of hydrogen was passed through the suspension for 2 h. The suspension was filtered through Celite and the solvent was removed under reduced pressure. 36 (273 mg, 0.48 mmol, quant.) was obtained as a yellow solid and was used in the next step without further purification steps.

¹H NMR (400 MHz, CDCl₃): δ=3.89-1.87 (br, 24H), 1.46 (s, 27H) ppm.

The spectroscopic data are in agreement with the literature.^{[16]}

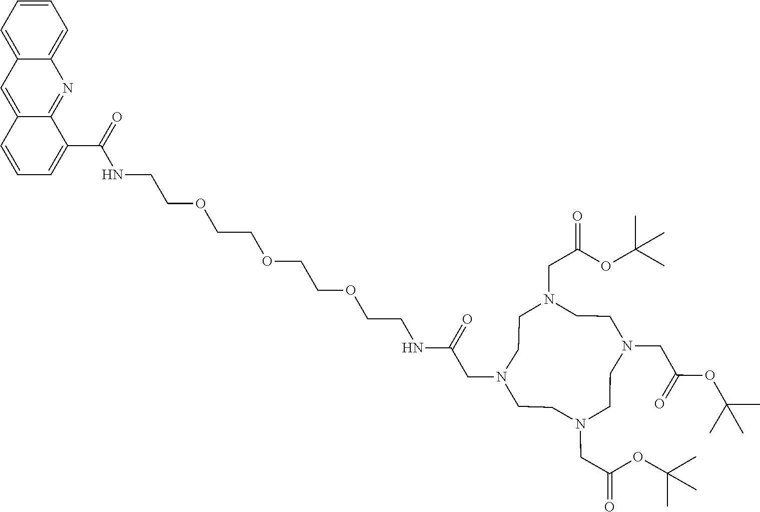 [SMILES: CC(C)(C)OC(=O)CN1CCN(CC(=O)NCCOCCOCCOCCNC(=O)c2cccc3cc4ccccc4nc23)CCN(CC(=O)OC(C)(C)C)CCN(CC(=O)OC(C)(C)C)CC1]

### Tri-Tert-Butyl 2,2′ 2″-(10-(1-(acridin-4-yl)-1,15-dioxo-5,8,11-trioxa-2,14-diazahexadecan-16-yl)-1,4,7,10-tetraazacyciododecane-14,7-triyl)triacetate 37

33 (129 mg, 0.22 mmol), HATU (85 mg, 0.22 mmol) and DIPEA (90 μl) were dissolved in CH₂Cl₂ (1.8 ml) at room temperature. The solution was stirred for 10 min and 36 (69 mg, 0.17 mmol) was added to the reaction. The reaction was stirred for 15 h. The reaction was stopped by addition of H₂O and the aqueous phase was extracted with CH₂Cl₂. The combined organic phase was washed with saturated NH₄Cl, saturated NaHCO₃ and saturated NaCl solution, successively. The organic phase was dried over Na₂SO₄, filtered and the solvent was removed under reduced pressure. The crude product was purified by column chromatography (PE/EA: 5/1-1/3). 37 (73 mg, 0.07 mmol, 44%) was isolated as a yellow oil.

¹H NMR (400 MHz, CDCl₃): δ=12.26 (s, 1H), 8.97 (s, 1H), 8.93 (dd, J=7.1, 1.5 Hz, 1H), 8.24 (dd, J=17.9, 8.5 Hz, 2H), 8.10 (d, J=8.5 Hz, 1H), 7.90 (t, J=7.8 Hz, 1H), 7.67 (dt, J=15.1, 7.6 Hz, 2H), 6.55 (s, 1H), 4.05-3.86 (m, 4H), 3.81 (dd, J=5.9, 3.2 Hz, 2H), 3.74 (dd, J=5.8, 3.3 Hz, 2H), 3.66 (dd, J=5.8, 3.4, 2H), 3.57 (dd, J=5.8, 3.4 Hz, 2H), 3.53 (bs, 2H), 3.40 (d, J=5.5 Hz, 2H), 3.24 (dd, J=7.5, 2.9 Hz, 10H), 2.62-1.78 (broad set of signals with a total integral of 14H), 1.46 (d, J=7.2 Hz, 27H).

¹³C NMR (101 MHz, CDCl₃): δ=207.1, 172.5, 171.6, 166.3, 147.6, 146.2, 138.0, 135.2, 132.8, 131.6, 129.0, 128.3, 126.9, 126.5, 126.1, 125.3, 81.9, 70.6, 70.5, 70.4, 70.3, 70.2, 69.5, 47.5, 38.6, 31.0, 28.0, 27.9, 8.8.

R_{f} (PE/EA=1/3): 0.2

HRMS (ESI): m/z calculated for C₅₀H₇₈N₇O₁₁ [M+H]⁺: 952.5759, found: 952.5758

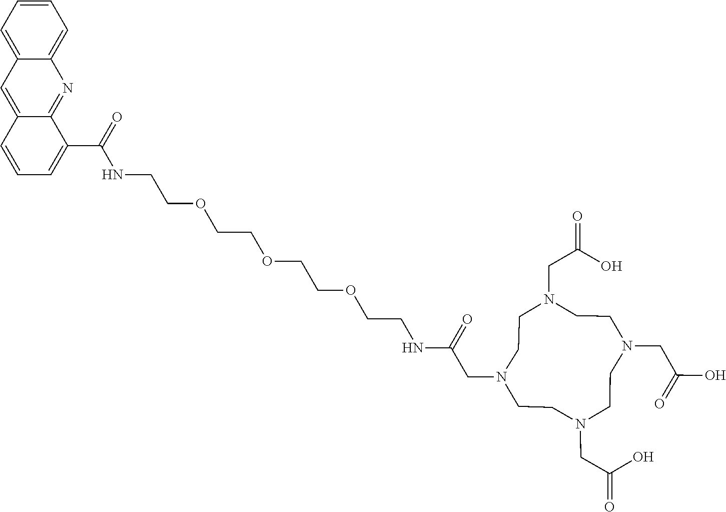 [SMILES: O=C(O)CN1CCN(CC(=O)O)CCN(CC(=O)NCCOCCOCCOCCNC(=O)c2cccc3cc4ccccc4nc23)CCN(CC(=O)O)CC1]

### 2,2′,2″-(10-(1-(acridin-4-yl)-1,15-dioxo-58,11-trioxa-2,4diazahexadecan-16-yl)-1,4,7,10-38

37 (57 mg, 0.06 mmol) was dissolved in CH₂Cl₂/TFA (3/1, 600 μl) at room temperature. The solution was allowed to stir for 36 h and subsequently the solvents were removed in vacuo. 38 (41 mg, 0.05 mmol, 87%) was isolated as a yellow oil and was used without further purification steps.

¹H NMR (400 MHz, CDCl₃): δ=9.25 (s, 1H), 8.87 (dd, J=7.1, 1.6, 1H), 8.43-8.37 (m, 1H), 8.33 (d, J=8.8 Hz, 1H), 8.24 (d, J=8.4 Hz, 1H), 7.99 (dd, J=8.6, 6.8 Hz, 1H), 7.83-7.69 (m, 2H), 4.16-3.40 (m, 30H), 1.60-1.06 (m, 6H).

¹³C NMR (101 MHz, CDCl₃): δ=166.7, 135.0, 133.3, 132.3, 128.4, 127.8, 126.9, 126.6, 126.2, 125.0, 117.9, 115.0, 70.3, 70.2, 70.1, 69.6, 69.4, 68.8, 39.5, 38.9, 7.8.

HRMS (ESI): m/z calculated for C₃₈H₅₃N₇O₁₁Na[M+Na]⁺: 806.3701, found: 806.3709

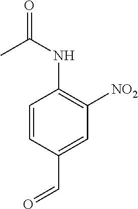

### N-(4-formyl-2-nitrophenyl)acetamide 39

Conc. H₂SO₄ (280 μl) was placed at 0° C. and HNO₃ (fuming, 70 μl) was added dropwise. To this solution 4-acetamino-3-nitrobenzaldehyde (100 mg, 0.61 mmol) was added in portions and the reaction was allowed to stir for 30 minutes at this temperature. The reaction was stopped by adding Na₂CO₃ (saturated solution) and the aqueous phase was extracted with CH₂Cl₂. The combined organic phases were dried over Na₂SO₄, filtered and the solvent was removed under reduced pressure. The crude product was purified by column chromatography (PE/EE=3/1-1/1). 39 (74 mg, 0.36 mml, 58%) was isolated as a yellow solid.

¹H NMR (400 MHz, CDCl₃): δ=10.66 (s, 1H), 10.01 (s, 1H), 9.06 (d, J=8.8, 1H), 8.77 (d, J=2.0, 1H), 8.18 (dd, J=8.8, 2.0, 1H), 2.38 (s, 3H).

The analytical data are in agreement with the literature^{[8]}.

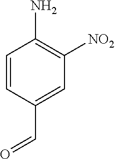 [SMILES: Nc1ccc(C=O)cc1[N+](=O)[O-]]

### 4-amino-3-nitrobenzaldehyde

39 (1.99 g, 9.6 mmol) was added to H₂SO₄ (10 vol. %, 9.5 ml) at room temperature. The resulting suspension was allowed to stir at 100° C. for 1.5 h. The reaction was stopped by the addition of The reaction was stopped by addition of aq. Na₂CO₃ (saturated solution) and the aqueous phase was extracted with CH₂Cl₂. The combined organic phase was dried over Na₂SO₄, filtered and the solvent was removed under reduced pressure. 40 (1.52 g, 9.1 mmol, 96%) was isolated as a yellow solid and used without further purification.

¹H NMR (400 MHz, DMSO-d₆): δ=9.76 (s, 1H), 8.57 (d, J=2.0 Hz, 1H), 8.36-8.18 (m, 2H), 7.80 (dd, J=8.9, 2.0 Hz, 1H), 7.12 (d, J=8.8 Hz, 1H).

The analytical data are in agreement with the literature^{[9]}

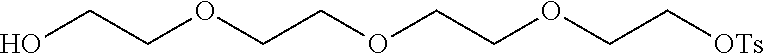 [SMILES: Cc1ccc(S(=O)(=O)OCCOCCOCCOCCO)cc1]

### 2-(2-(2-(2-hydroxyethoxy)ethoxy)ethoxy)ethyl 4-methylbenzenesulfonate 41

Tetraethylene glycol (1.99 g, 10.3 mmol) was dissolved at 0° C. in CH₂Cl₂ (100 ml). Tosyl chloride (2.15 g, 11.3 mmol), Ag₂O (3.6 g, 15.4 mmol) and KI (340 mg, 2.05 mmol) were added to the solution. The reaction suspension was stirred for 20 min at this temperature.

The suspension was then filtered through Celite and the solvent was removed in vacuo. The crude product was purified by column chromatography (PE/EE=1/1-1/3-0/100). 41 (2.2 g, 6.5 mmol, 63%) was isolated as a colourless oil.

¹H NMR (400 MHz, CDCl₃): δ=7.78 (d, J=8.3 Hz, 2H), 7.32 (d, J=7.9 Hz, 2H), 4.16-4.13 (m, 2H), 3.69-3.57 (m, 14H), 2.43 (s, 3H).

The analytical data are in agreement with the literature^{[10]}.

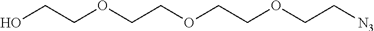 [SMILES: [N-]=[N+]=NCCOCCOCCOCCO]

### 2-(2-(2-(2-azidoethoxy)ethoxy)ethoxy)ethan-1-ol 42

41 (1.2 g, 3.5 mmol) was dissolved in DMF (7 ml) at room temperature. NaN₃ (560 mg, 8.6 mmol) was added to the solution and the suspension was allowed to stir at 80° C. for 18 h. The solvent was removed under reduced pressure and the residue was taken up in water and CH₂Cl₂. The organic phase was washed with water and the combined aqueous phases were back-extracted with CH₂Cl₂. The combined organic phase was dried over Na₂SO₄, filtered and the solvent was removed under reduced pressure. 42 (734 mg, 3.3 mmol, 97%) was isolated as a colourless oil.

¹H NMR (400 MHz, CDCl₃): δ=3.80-3.73 (m, 1H), 3.72-3.68 (m, 5H), 3.66-3.61 (m, 1H), 3.42 (t, J=5.1 Hz, 1H).

The analytical data are in agreement with the literature^{[11]}.

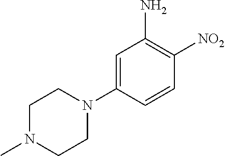 [SMILES: CN1CCN(c2ccc([N+](=O)[O-])c(N)c2)CC1]

### 5-(4-methylpiperazin-1-yl)-2-nitroaniline 43

5-Chloro-2-nitroaniline (250 mg, 1.5 mmol), K₂CO₃ (300 mg, 2.2 mmol) and methylpiperazine (193 μl, 1.7 mmol) were suspended in DMF (870 μl) and stirred at 120° C. for 15 h. The suspension was then taken up in vacuum. After this time the suspension was concentrated in vacuo and the residue was taken up in water and CH₂Cl₂. The organic phase was washed with water and dried over Na₂SO₄. The solvent was removed under reduced pressure and the crude product was purified by column chromatography (CH₃OH in CH₂Cl₂: 1%-6%-10%). 43 (287 mg, 1.2 mmol, 84%) was isolated as a yellow solid.

¹H NMR (400 MHz, CDCl₃): δ=8.03 (d, J=9.8, 1H), 6.30 (dd, J=9.7, 2.6 Hz, 1H), 6.17 (s, 2H), 5.97 (d, J=2.6 Hz, 1H), 3.39 (t, J=5.1 Hz, 4H), 2.54 (t, J=5.1 Hz, 4H), 2.36 (s, 3H).

The analytical data are in agreement with the literature.^{[12]}

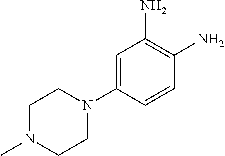

### 4-(4-methylpiperazin-1-yl)benzene-1,2-diamine 44

43 (192 mg, 0.8 mmol) was dissolved in EtOAc/CH₃OH (4/1, 7.5 ml) at room temperature. Palladium on activated carbon (10%, 10 mg) was added to the solution and a stream of hydrogen was passed through the suspension for 2 h. The suspension was then filtered through Celite and the solvent was removed. The suspension was then filtered through Celite and the solvent was removed under reduced pressure. 44 (163 mg, 0.8 mmol, 97%) was isolated as brown oil and further reacted without further purification.

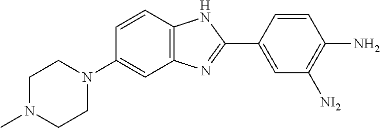

### 4-(5-(4-methylpiperazin-1-yl)-1H-benzo[d]imidazol-2-yl)-2-nitroaniline 45

40 (390 mg, 2.35 mmol), 44 (508 mg, 2.47 mmol) and Na₂S₂O₅ (446 mg, 2.35 mmol) were dissolved in DMF (4.6 ml) at room temperature. The resulting suspension was allowed to stir at 70° C. for 1.5 h. The solvent was removed in vacuo and the crude product was purified by column chromatography (20% CH₃OH in CH₂Cl₂). 45 (437 mg, 1.24 mmol, 53%) was isolated as an orange solid.

¹H NMR (400 MHz, DMSO-d₆): δ=12.65 (s, 1H), 8.76 (s, 1H), 8.15 (dd, J=8.9 Hz, 2.1, 1H), 7.78 (s, 2H), 7.43 (s, 1H), 7.15 (d, J=8.9 Hz, 1H), 6.93 (d, J=9.0 Hz, 1H), 3.17 (s, 4H), 2.64 (s, 4H), 2.34 (s, 3H).

¹³C NMR (101 MHz, DMSO-d₆): δ=148.0, 147.2, 141.4, 136.6, 133.8, 130.5, 123.3, 120.3, 118.5, 54.9, 49.8, 49.0, 46.0, 45.5.

R_{f} (10% CH₃OH in CH₂Cl₂)=0.25

HRMS (ESI): m/z calculated for C₁₈H₂₀N₆O₂Na [M+Na]⁺: 375.1545, found: 375.1552.

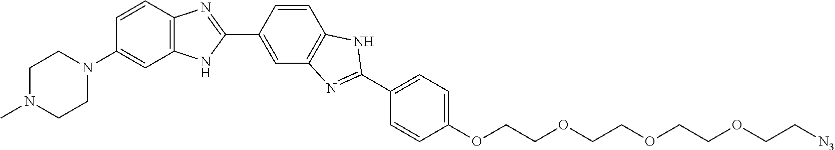 [SMILES: CN1CCN(c2ccc3nc(-c4ccc5[nH]c(-c6ccc(OCCOCCOCCOCCN=[N+]=[N-])cc6)nc5c4)[nH]c3c2)CC1]

### 2′-(4-(2-(2-(2-(2-azidoethoxy)ethoxy)ethoxy)ethoxy)phenyl)-6-(4-methylpiperazin-1-yl)-1H,1′H-2,5′-bibenzo[d]imidazole 46

45 (350 mg, 1.08 mmol) and 47 (349 mg, 1.08 mmol) were dissolved in DMF (2.1 ml) at room temperature. Na₂S₂O₅ (205 mg, 1.08 mmol) was added to the solution and the resulting suspension was allowed to stir at 70° C. for 18 h. The solvent was removed under reduced pressure and the crude product was purified by column chromatography (20% CH₃OH in CH₂Cl₂). 46 (498 mg, 0.76 mmol, 74%) was isolated as a yellow solid.

¹H NMR (400 MHz, CD₃OD): δ=8.27 (s, 1H), 8.06 (d, J=8.8 Hz, 2H), 7.96 (dd, J=8.5, 1.7 Hz, 1H), 7.70 (d, J=8.5 Hz, 1H), 7.53 (d, J=8.8 Hz, 1H), 7.16 (d, J=2.2 Hz, 1H), 7.11 (d, J=8.9 Hz, 2H), 7.07 (dd, J=8.8, 2.2 Hz, 1H), 4.25-4.16 (m, 2H), 3.92-3.86 (m, 2H), 3.76-3.62 (m, 10H), 3.41-3.34 (m, 2H), 3.28 (t, J=5.1 Hz, 5H), 2.82 (t, J=5.0 Hz, 4H), 2.49 (s, 3H).

¹³C NMR (101 MHz, CD₃OD): δ=161.1, 153.9, 152.4, 148.0, 128.2, 124.3, 121.7, 121.1, 115.1, 114.8, 70.4, 70.3, 70.1, 69.7, 69.3, 67.4, 54.6, 50.4, 50.1, 44.3.

R_{f} (20% CH₃OH in CH₂Cl₂)=0.15

HRMS (ESI): m/z calculated for C₃₃H₄₀N₉O₄ [M+Na]⁺: 626.3203, found: 626.3199.

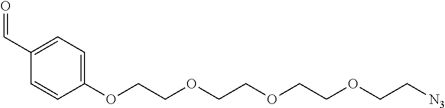 [SMILES: [N-]=[N+]=NCCOCCOCCOCCOc1ccc(C=O)cc1]

### 4-(2-(2-(2-(2-azidoethoxy)ethoxy)ethoxy)ethoxy)benzaldehyde 47

42 (1257 g, 5.7 mmol), 4-hydroxybenzaldehyde (910 mg, 7.5 mmol) and triphenylphosphane (1298 mg, 7.5 mmol) were dissolved at 0° C. in THF (30 ml). Diethyl azodicarboxylate (1.2 ml, 7.5 mmol) was added to the solution. The reaction was stirred for 18 h while allowing it to warm to room temperature. The solvent was removed under reduced pressure and the residue was taken up in water and CH₂Cl₂. The organic phase was washed with water and then dried over Na₂SO₄, filtered and the solvent was removed under reduced pressure. The crude product was purified by column chromatography (PE/EE=3/1-1/1). 47 (1588 mg, 4.9 mmol, 83%) was obtained as a colourless oil.

¹H NMR (400 MHz, CDCl₃): δ=9.85 (s, 1H), 7.79 (d, J=8.8 Hz, 2H), 6.99 (d, J=8.8 Hz, 2H), 4.26-4.08 (m, 2H), 3.93-3.83 (m, 2H), 3.78-3.58 (m, 10H), 3.34 (t, J=5.0 Hz, 2H).

¹³C NMR (101 MHz, CDCl₃): δ=190.8, 163.8, 131.9, 130.0, 114.8, 70.8, 70.7, 70.6, 70.6, 70.0, 69.4, 67.7, 50.6.

R_{f} (PE/EE=1/1): 0.2

HRMS (ESI): m/z calculated for C₁₅H₂₁N₃O₅Na [M+Na]⁺: 346.1379, found: 346.1385

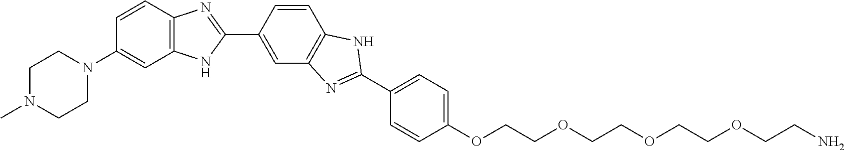 [SMILES: CN1CCN(c2ccc3nc(-c4ccc5[nH]c(-c6ccc(OCCOCCOCCOCCN)cc6)nc5c4)[nH]c3c2)CC1]

### 2-(2-(2-(2-(4-(6-(4-methylpiperazin-1-yl)-1H,1′H-[2,5′-bibenzo[d]imidazol]-2′-yl)phenoxy)ethoxy)ethoxy)ethoxy)ethan-1-amine 48

47 (40 mg, 0.06 mmol) was dissolved at room temperature in degassed THF/H₂O (5/1, 570 μl). PMe₃ (1M in THF, 0.06 mmol, 65 μl) was added to the solution and the reaction was allowed to stir for 1 h. The solvents were then removed under reduced pressure. 48 (38 mg, quant.) was isolated as a yellow solid and was used without further purification steps.

¹H NMR (400 MHz, CD₃OD): δ=8.33-8.25 (m, 1H), 8.01-7.92 (m, 3H), 7.86-7.83 (m, 1H), 7.58-7.53 (m, 1H), 7.25-7.23 (m, 1H), 7.17-7.14 (m, 1H), 7.09-7.02 (m, 2H), 4.13-4.08 (m, 2H), 3.84-3.76 (m, 5H), 3.60-3.55 (m, 9H), 3.21-3.17 (m, 5H), 3.10-3.07 (m, 2H), 2.99 (t, J=4 Hz, 2H), 2.87 (s, 3H), 1.43 (d, J=6 Hz, 2H) ppm,

¹³C NMR (101 MHz, CD₃OD): δ=164.7, 150.8, 149.4, 134.5, 131.2, 127.9, 125.5, 120.8, 119.6, 116.9, 116.5, 115.7, 114.9, 100.8, 71.7, 71.5, 71.5, 71.2, 70.5, 69.2, 67.8, 62.2, 54.5, 43.6, 40.6 ppm.

HRMS (ESI): m/z calculated for C₃₃H₄₂N₇O₄ [M+H]⁺: 600.3298, found: 600.3298

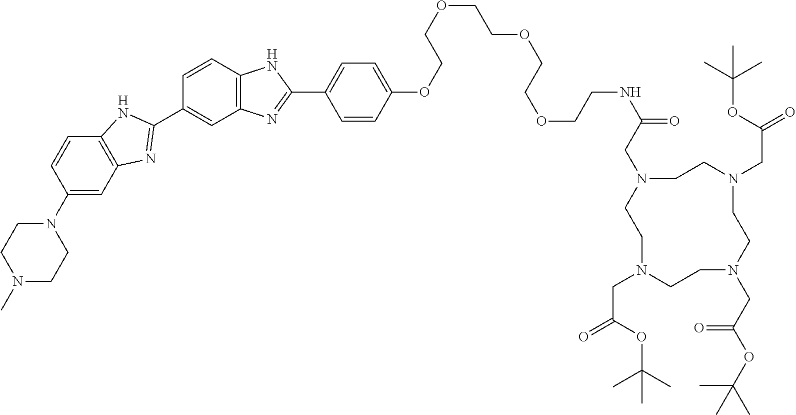 [SMILES: CN1CCN(c2ccc3[nH]c(-c4ccc5[nH]c(-c6ccc(OCCOCCOCCOCCNC(=O)CN7CCN(CC(=O)OC(C)(C)C)CCN(CC(=O)OC(C)(C)C)CCN(CC(=O)OC(C)(C)C)CC7)cc6)nc5c4)nc3c2)CC1]

### Tri-tert-butyl 2-2′,2″-(10-(14-(4-(5-(4-methylpiperazin-1-yl)-1H,1′H-[2,5′-bibenzo[d]imidazol]-2′-yl)phenoxy)-2-oxo-6,9,12-trioxa-3-azatetradecyl)-1,4,7,10-tetraazacyclododcecane-1,4,7-triyl)triacetate 49

36 (44 mg, 0.08 mmol), DIPEA (32 μl, 0.2 mmol) and HATU (0.08 mmol) were dissolved in DMF (300 μl) at room temperature and stirred for 15 min. To the solution was added a solution of 48 (38 mg, 0.06 mmol) in DMF (300 μl) and the resulting solution was allowed to stir for 18 h at room temperature. The solvent was subsequently removed under reduced pressure and the crude product was purified by column chromatography (C18 cartridge, ACN+0.05% TFA/H₂O+0.05% TFA, 5-95%). 49 (52 mg, 0.05 mmol, 62%) was obtained as a yellow solid.

¹H NMR (400 MHz, CD₃OD): δ=8.56 (s, 1H), 8.20 (d, J=4 Hz, 3H), 8.04 (d, J=4 Hz, 1H), 7.98 (s, 2H), 7.76 (d, J=4 Hz, 1H), 7.44 (d, J=4 Hz, 1H), 7.36 (s, 1H), 7.28 (d, J=4 Hz, 2H), 4.30 (t, J=4 Hz, 2H), 3.98 (d, J=8 Hz, 3H), 3.91 (t, J=4 Hz, 3H), 3.75-3.64 (m, 14H), 3.57 (brs, 4H), 3.22 (t, J=12 Hz, 4H), 3.01 (s, 3H), 3.00 (s, 7H), 2.86 (s, 7H), 2.03 (s, 1H), 1.94 (s, 1H), 1.54-1.47 (m, 27H), 1.31-1.29 (m, 3H) ppm,

¹³C NMR (101 MHz, CD₃OD): δ=164.9, 164.6, 161.5, 161.2, 155.0, 151.0, 150.1, 134.7, 131.1, 128.2, 125.3, 120.0, 118.8, 117.0, 115.7, 101.1, 71.8, 71.7, 71.6, 71.3, 70.6, 69.2, 54.6, 43.6, 37.0, 31.6, 28.5 ppm.

HRMS (ESI): m/z calculated for C₆₁H₉₁N₁₁O₁₁Na [M+Na]⁺: 1176.6797, found: 1176.6799

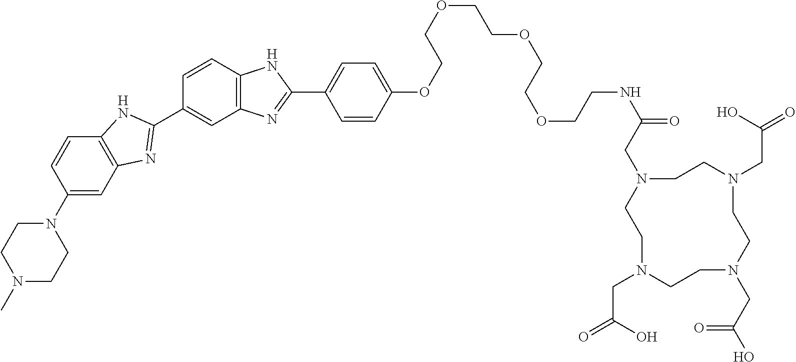 [SMILES: CN1CCN(c2ccc3[nH]c(-c4ccc5[nH]c(-c6ccc(OCCOCCOCCOCCNC(=O)CN7CCN(CC(=O)O)CCN(CC(=O)O)CCN(CC(=O)O)CC7)cc6)nc5c4)nc3c2)CC1]

### 2,2′,2″-(10-(14-(4-(5-(4-methylpiperazin-1-yl)-1H,1′H-[2,5′-bibenzo[d]imidazol]-2′-yl)phenoxy)-2-oxo-6,9,12-trioxa-3-azatetradecyl)-1,4,7,10-tetraazacyclododecane-1,4,7-triyl)triacetic Acid 50

49 (16 mg, 0.01 mmol) was dissolved in CH₂Cl₂/TFA(7/3, μl) at room temperature. The reaction was allowed to stir for 18 h and subsequently the solvents were removed under reduced pressure. The crude product was purified by column chromatography (C18 cartridge, ACN+0.05% TFA/H₂O+0.05% TFA, 5-95%). 50 (10 mg, 0.01 mmol, 73%) was isolated as a yellow solid.

¹H NMR (400 MHz, DMSO-d₆): δ=8.34 (s, 1H), 8.17 (d, J=8.3, 1H), 8.02 (d, J=8.5, 1H), 7.70 (d, J=8.3, 1H), 7.52 (d, J=8.7, 1H), 7.22-7.09 (m, 2H), 7.02 (d, J=8.7, 1H), 4.29-4.14 (m, 2H), 3.87-3.76 (m, 2H), 3.69-2.99 (bs, 58H), 2.87 (s, 3H), 2.54 (s, 1H) ppm,

¹³C NMR (101 MHz, DMSO-d₆): δ=176.7, 174.9, 160.6, 158.6, 158.4, 158.2, 157.9, 132.7, 129.6, 128.8, 126.9, 122.7, 121.4, 119.0, 116.6, 115.4, 114.2, 72.9, 70.4, 70.3, 70.2, 70.0, 69.3, 67.9, 66.2, 63.5, 53.1, 49.1, 47.9, 42.6, 31.5, 28.7, 22.6, 20.9, 19.7, 14.4 ppm.

HRMS (ESI): m/z calculated for C₄₉H₆₈N₁₁O₁₁ [M+H]⁺: 985.5022, found: 985.5020

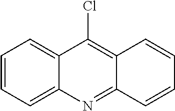 [SMILES: Clc1c2ccccc2nc2ccccc12]

### 9-chloroacridine 51

Acridone (600 mg, 3.07 mmol) was suspended in freshly distilled SOCl₂ (3 ml). DMF (30 μl, 0.37 mmol) was added to the suspension and the reaction was allowed to stir at 80° C. for 2 h. The solvents were removed in vacuo and the remaining residue was taken up in CHCl₃ and placed in a cold ammonia solution. The suspension was extracted with CHCl₃ and the combined organic phases were dried over Na₂SO₄, filtered and the solvent was removed under reduced pressure. 51 (620 mg, 2.90 mmol, 94%) was obtained as a yellow solid and used without further purification steps.

¹H NMR (400 MHz, CDCl₃): δ=8.44 (dd, J=8.8, 1.4 Hz, 2H), 8.23 (dt, J=8.8, 1.3 Hz, 2H), 7.82 (ddd, J=8.8, 6.6, 1.4 Hz, 2H), 7.64 (ddd, J=8.8, 6.6, 1.2 Hz, 2H).

¹³C NMR (101 MHz, CDCl₃): δ=206.9, 148.9, 130.5, 129.7, 126.8, 124.6, 124.2.

HRMS (ESI): m/z calculated for C₁₃H₉NCI[M+H]⁺: 214.0424, found: 214.0429

R_{f} (PE/EE=5/1)=0.4

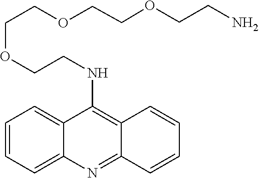 [SMILES: NCCOCCOCCOCCNc1c2ccccc2nc2ccccc12]

### N-(2-(2-(2-(2-azidoethoxy)ethoxy)ethoxy)ethyl)acridin-9-amine 52

51 (112 mg, 0.52 mmol) was dissolved in phenol (460 μl, 5.24 mmol) at 50° C. To the solution was added a solution of 19 (114 mg, 0.52 mmol) in phenol (460 μl, 5.24 mmol) at the same temperature. The reaction was allowed to stir at 120° C. for 30 min. After this time, the reaction was cooled to room temperature and the reaction mixture was taken up in CH₂Cl₂ and transferred to a silica column. The product was eluted with CH₃OH in CH₂Cl₂ (6%-10%). 52 (186 mg, 0.47 mmol, 90%) was obtained as a yellow solid.

¹H NMR (400 MHz, CDCl₃): δ=9.17 (s, 1H), 8.48 (dd, J=8.8, 1.4 Hz, 2H), 8.14 (dt, J=8.8, 1.3 Hz, 2H), 7.51 (ddd, J=8.8, 6.6, 1.4 Hz, 2H), 7.24 (ddd, J=8.8, 6.6, 1.2 Hz, 2H), 4.36 (t, J=5.3, 2H), 3.84-3.79 (m, 2H), 3.76-3.70 (m, 2H), 3.68-3.65 (m, J=5.0, 1.9, 2H), 3.63-3.55 (m, 4H), 3.32 (t, J=5.0, 2H).

¹³C NMR (101 MHz, CDCl₃): δ=157.1, 140.0, 133.8, 124.9, 123.2, 119.9, 112.4, 70.6, 70.6, 70.5, 70.0, 69.1, 60.4, 53.5, 50.6, 48.5, 21.1, 14.2.

HRMS (ESI): m/z calculated for C₂₁H₂₆N₅O₃[M+H]⁺: 396.2036, found: 396.2041

R_{f} (8% CH₃OH in CH₂Cl₂)=0.25

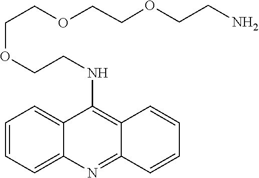 [SMILES: NCCOCCOCCOCCNc1c2ccccc2nc2ccccc12]

### N-(2-(2-(2-(2-aminoethoxy)ethoxy)ethoxy)ethyl)acridin-9-amine 53

52 (57 mg; 0.14 mmol) was dissolved in a degassed mixture of THF/H₂O (4/1; 1.5 ml) at room temperature. Trimethylphosphine (1 M in THF; 0.17 mmol; 170 μl) was added to the solution and stirring was continued for 3 h. After this time, the solvent was removed under reduced pressure. 53 (62 mg, quant.) was isolated as colourless oil and was used in the next reaction without further purification.

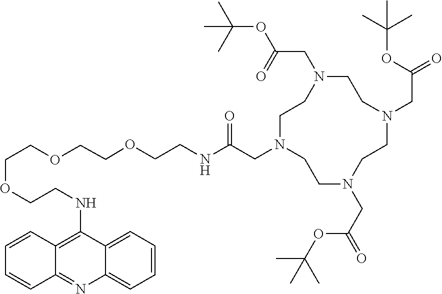 [SMILES: CC(C)(C)OC(=O)CN1CCN(CC(=O)NCCOCCOCCOCCNc2c3ccccc3nc3ccccc23)CCN(CC(=O)OC(C)(C)C)CCN(CC(=O)OC(C)(C)C)CC1]

### Tri-Tert-Butyl 2,2′,2″-(10-(14-(acridin-9-ylamino)-2-oxo-6,9,12-trioxa-3-azatetradecyl)-1,4,7,10-tetraazacyclododecane-1,4,7-triyl)triacetate 54

36 (106 mg, 0.19 mmol), HATU (71 mg, 0.19 mmol) and DIPEA (74 μl, 0.43 mmol) were dissolved in CH₂Cl₂ (2 ml) at room temperature. The solution was allowed to stir for 15 min and then a solution of 53 (53 mg, 0.14 mmol) in CH₂Cl₂ (2 ml) was added. The reaction was allowed to stir for 18 h. The solvent was removed under reduced pressure and the crude product was purified by column chromatography (CH₃OH in CH₂Cl₂: 2%). 54 (10 mg, 0.01 mmol, 7%) was isolated as a yellow oil.

¹H NMR (400 MHz, CD₃OD): δ=8.46 (d, J=7.3 Hz, 2H), 8.28 (d, J=8.2 Hz, 2H), 7.76 (t, J=7.8 Hz, 2H), 4.61-2.56 (m, 44H), 1.51 (d, J=32.7 Hz, 27H)

¹³C NMR (101 MHz, CD₃OD): δ=164.1, 163.4, 160.7, 160.4, 160.0, 159.7, 157.3, 134.2, 131.6, 130.7, 127.7, 126.8, 126.8, 122.0, 120.5, 117.68, 116.0, 114.7, 113.2, 111.8, 70.1, 70.1, 70.1, 69.9, 69.8, 69.0, 67.4, 39.0, 38.8, 37.5, 35.6, 27.1, 27.1.

HRMS (ESI): m/z calculated for C₄₉H₇₇N₇O₁₀Na[M+Na]⁺: 946.5630, found: 946.5625

R_{f} (5% CH₃OH in CH₂Cl₂)=0.45

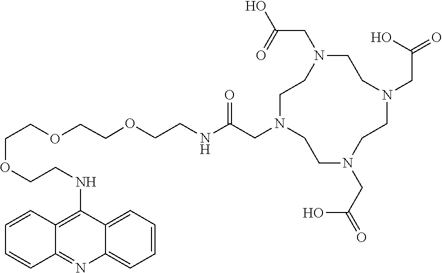 [SMILES: O=C(O)CN1CCN(CC(=O)O)CCN(CC(=O)NCCOCCOCCOCCNc2c3ccccc3nc3ccccc23)CCN(CC(=O)O)CC1]

### 2,2′,2″-(10-(14-(acridin-9-ylamino)-2-oxo-6,9,12-trioxa-3-azatetradecyl)-1,4,7,10-tetraazacyclododecane-1,4,7-triyl)triacetic Acid 55

54 (120 mg, 0.13 mmol) was dissolved in a mixture of TFA/CH2Cl2 (50/50, 1.3 ml) at room temperature and allowed to stir for 24 h. The solvent was removed under reduced pressure and the crude product was purified by column chromatography (C18 cartridge, ACN+0.05% TFA/H2O+0.05% TFA, 5-95%). 55 (84 mg, 0.11 mmol, 98%) was isolated as a colourless oil.

¹H NMR (400 MHz, CD₃OD): δ=8.61 (d, J=8.7 Hz, 2H), 8.01 (t, J=7.7 Hz, 2H), 7.87 (d, J=8.6 Hz, 2H), 7.60 (t, J=7.8 Hz, 2H), 4.61-2.74 (m, 46H) ppm,

¹³C NMR (400 MHz, CD₃OD): δ=160.75, 160.39, 158.98, 150.67, 135.17, 123.58, 118.27, 114.83, 70.08, 69.64, 68.93, 68.45, 48.97, 38.84, 13.01 ppm.

HRMS (ESI): m/z calculated für C₄₉H₇₇N₇O₁₀Na[M+Na]⁺: 756.3932, found: 756.3935

### Biology

Three different methods were chosen for cultivating biofilms. Which method was used for which experiment can be found in the instructions for the respective experiment.

### Method I: Cultivation of a Biofilm in a 96-Well Plate

10 ml of culture medium was inoculated with a cryoculture of the corresponding bacterial strain and incubated for 24 h at the appropriate temperature at 180 rpm in a shaking incubator. The temperatures selected for the strains used, as well as the culture media used, are shown in Table 1.

TABLE 1
Culture media and incubation temperatures
for the used bacteria strains

| Bacteria strain | Culture medium | Incubation temperature[° C.] |
|---|---|---|
| P. fluorescens | LB-Medium | 30 |
| P. aeruginosa | LB-Medium | 37 |
| S. aureus | TSB-Medium | 37 |
| E. coli | LB-Medium | 37 |

The obtained precultures were diluted with fresh medium to OD600=0.075 and 200 μl of the diluted precultures were added to 96-well plates. The filled plates were incubated for another 24 h at the appropriate temperature without stirring. After this time, the supernatant was removed and the residue was washed once with PBS to remove unbound bacteria. The biofilms were air dried and the existence of the biofilms was confirmed by staining with a 0.1% crystal violet solution. For this purpose, the biofilms were incubated with 200 μl of the crystal violet solution for 10 min at room temperature. The supernatant was removed and the residue was washed with water. The areas where a biofilm formed were left stained violet.

### METHOD II: Cultivation of a Biofilm in an Eppendorf Tube

The cultivation of the pre-culture was carried out analogously to the procedure described in method I. The obtained precultures were diluted with fresh medium to OD600=0.075 and 200 μl of the diluted precultures were added to microreaction tubes (1.5 ml, non-treated surface). The vessels were sealed and a hole was drilled in the lid with a cannula to ensure air exchange. The filled tubes were incubated for 24 h at the appropriate temperature without stirring. After this time, the supernatant was removed and the residue was washed once with PBS to remove unbound bacteria. The biofilms were air dried and the existence of the biofilms was confirmed by staining with a 0.1% crystal violet solution. For this purpose, the biofilms were incubated with 200 μl of the crystal violet solution for 10 min at room temperature. The supernatant was removed and the residue was washed with water. The sites where biofilm formed were left stained violet.

### Cultivation of Eukaryotic Cells

20 ml of culture medium was inoculated in a T75 flask with 106 cells of a cryoculture and incubated for 5 d at 37° C. in a 5% CO₂ atmosphere. The selected culture media are shown in Table 2.

T Culture media and incubation temperatures for the used bacteria strains.

| Cell strain | Culture medium |
|---|---|
| CHO | 89 vol. % Ham's F-12 nutrition mix, 10 vol. % FBS, 1 vol. % |
| | Gentamicin (c = 5 μg/ml) |
| THP-1 | 99 vol. % RPMI-1640 medium, 1 vol. % Gentamicin (c = 5 |
| | μg/ml) |

During the cultivation period, the CHO cells became adherent, the THP-1 cells remained in planktonic form. For further use, the adherent cells were detached from the bottom of the bottle. For this purpose, the supernatant was first aspirated and the residue was rinsed once with PBS (10 ml). To detach the cells, the bottom of the bottle was covered with an Accutase solution (5 ml, 400-600 units/ml) and incubated at 37° C. for 5 min. To deactivate the enzyme, 5 ml of fresh medium was added to the suspension. The suspension was centrifuged (5 min, 300 rcf) and the supernatant was removed. The cells were resuspended with fresh medium and were further used in this form. The unbound cells were centrifuged without further steps and used analogously.

### Cell Permeability Assay

For cell permeability assays, 104 CHO cells (cultured as previously described) were seeded in 200 μl medium in a 96 well plate and incubated for 3 d at 37° C. in a 5% CO₂ atmosphere, during which time the cells re-adhered. After the time, the supernatant was removed and the cells were washed with PBS.

The prepared cells were incubated with a) 200 μl of a Hoechst 33342 solution (10 μM), b) 200 μl of a solution of 50 (10 μM) and c) 200 μl of a suspension of HO particles (c=1 mg/ml). The cells were incubated for 10 min at 37° C. in a 5% CO₂ atmosphere. Subsequently, the supernatants were removed, the residues were washed with PBS and the cells were examined in a Cytation 5 plate reader in brightfield and using a DAPI filter (ex/em: 377/50 nm/447/60 nm).

DNA Binding Assays with Molecular Probes 50 and 55

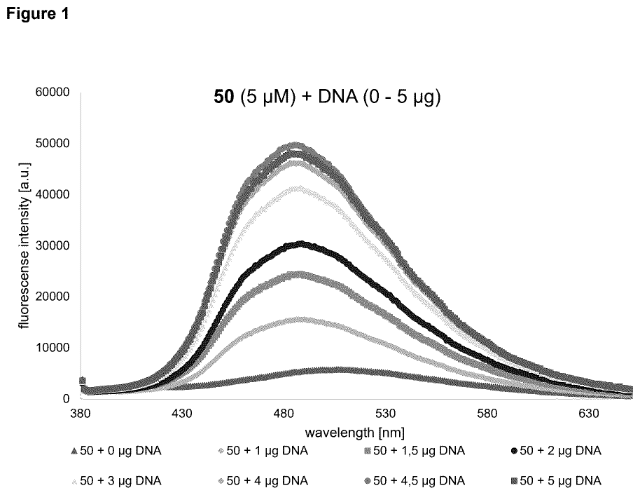

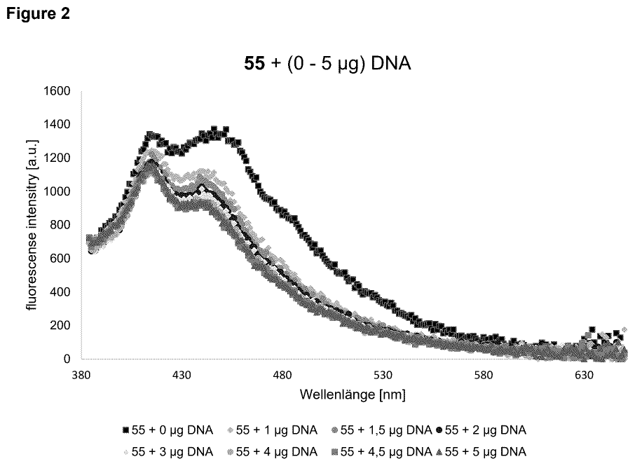

In a 96-well plate, an increasing amount of DNA (0-5 μg) from a stock solution was added to 200 μl of a 5 μM solution of the corresponding compound. The stock solution was prepared by dissolving salmon sperm DNA (Sigma Aldrich) in a DNA buffer solution (1 mM EDTA, 1 mM NaCl, 1 mM Tris, pH=7.4) to a concentration of c=1 mg/ml. The resulting solutions were incubated for 10 min at room temperature and subsequently examined for fluorescence in a Cytation 5 plate reader. The corresponding irradiation wavelengths result from the absorption maxima of the respective. All experiments were performed in triplicates and the results presented are the mean of each measurement point of the corresponding wavelengths. See FIGS. 1 and 2.

### Radiolabelling

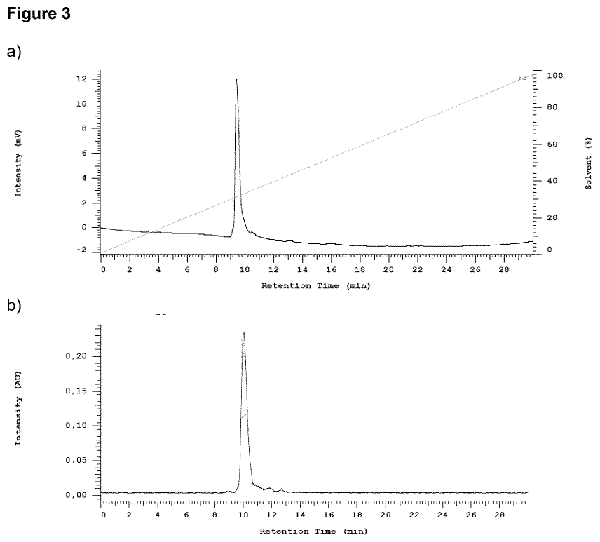

30 μl of a stock solution of 50 (1 mg/ml in 70 vol % EtOH) was dissolved in 300 μl of HEPES buffer. This solution was added to a ⁶⁸Ga solution (325 MBq) with a syringe. The mixture was incubated at 100° C. for 10 min and then the solution was diluted with 10 ml water, drawn up with a syringe and filtered through an HLB cartridge. The cartridge was washed once more with 5 ml water. 50* was eluted from the cartridge with 1 ml EtOH. The purity of the product obtained was determined by HPLC. 50* (194 MBq, 60%) was used without further purification steps. See FIG. 3.

### Radioincubation

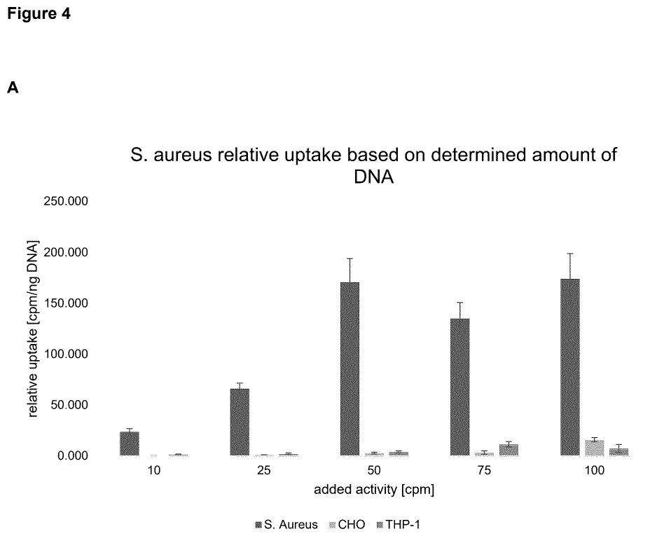

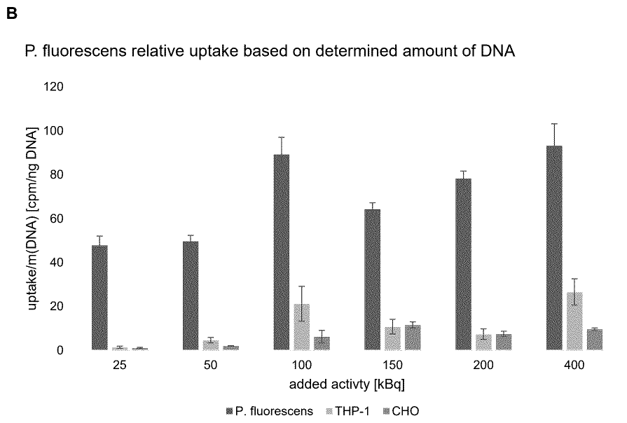

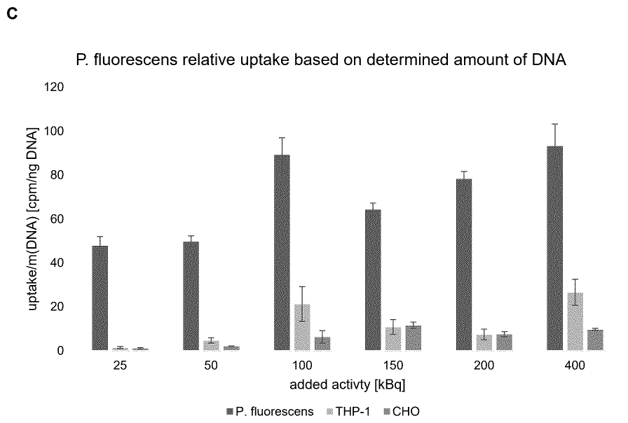

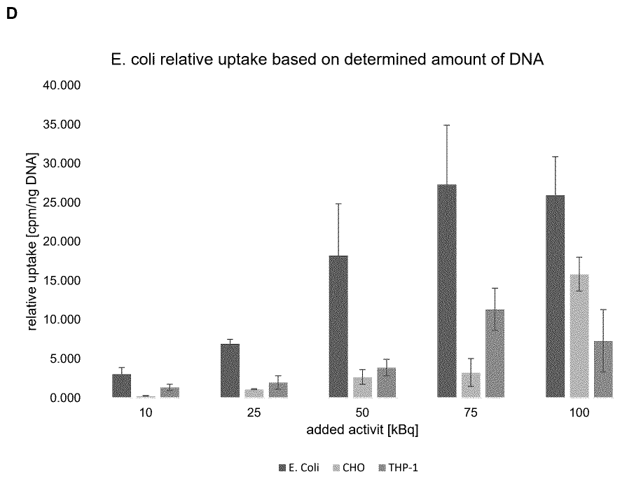

For the experiments, biofilms of the strains E. coli, P. fluorescens, P. aeruginosa and S. aureus were cultivated according to method II. The biofilms were covered with 200 μl of the corresponding medium (Table 1). In addition, 5·10⁵ CHO cells, and 5·10⁵ THP-1 cells were suspended in 200 μl of the appropriate medium (see table 2) in microreaction vials. An increasing amount of the active incubation solution was added to each sample (10-400 kBq). The incubation solution was obtained by adding the active probe to inactive PBS (c=1 MBq/ml or 10 MBq/ml). The covered cultures were incubated at 37° C. for 20 minutes. After this time, the solution was removed and the cultures were rinsed three times with PBS. To determine the residual activity, the biomaterial was lysed with SDS (2%) at 37° C. for 25 minutes. The lysate was transferred to a test tube and the remaining activities were determined in a γ-counter. To determine the background activity, uncultured microreaction vessels were additionally incubated with the same activities. The procedure with these vessels was analogous to the procedure with the cultured vessels. To determine the percentage uptake of the cultures, the total activity of the added activities was also determined in the γ-counter. All experiments were carried out in triplicates and the results presented correspond to the mean of each measuring point of the corresponding activity. See FIG. 4.

### REFERENCES

[1] US2004/6203, 2004, A1
[2] Kang et al.; Bioorganic and Medicinal Chemistry, 2003, vol. 11, #12, p. 2529-2539
[3] Okoth et al.; Beilstein Journal of Organic Chemistry, 2013, vol. 9, p. 608-612
[4] Segura et al.; Synthesis, 2001, #14, p. 2105-2112
[5] Rao et al.; Organic Letters, 2018, vol. 20, #6, p. 1680-1683
[6] Spicer et al.; Journal of Medicinal Chemistry, 1997, vol. 40, #12, p. 1919-1929
[7] Wang et al. Org. Biomol. Chem., 2015, 13, 6580-6586
[8] Tuccitto et al.; Chemistry—A European Journal, 2021, vol. 27, #55, p. 13715-13718
[9] Gabe et al.; Journal of the American Chemical Society, 2004, vol. 126, #10, p. 3357-3367
[10] Chirkin et al.; Angewandte Chemie—International Edition, 2017, vol. 56, #42, p. 13036-13040
[11] Cheng et al.; Chemical Communications, 2020, vol. 56, #18, p. 2787-2790
[12] Yan et al.; Journal of Medicinal Chemistry, 2016, vol. 59, #14, p. 6690-6708
[13] Kong et al.; Chemical Communications, 2022, vol. 58, #9, p. 1314-1317
[14] Peukert et al.; Journal of Medicinal Chemistry, 2021, vol. 64, #16, p. 12359-12378
[15] Strauch et al.; Journal of the American Chemical Society, 2011, vol. 133, #41, p. 16346-16349
[16] Winnett et al.; Inorganic Chemistry, 2020, vol. 59, #1, p. 118-127
[17] Learmonth et. al; Lancet, 2007, 370, p. 1508-19
[18] Kurtz et al.; Journal of Bone and Joint Surgery, 2007, vol. 89, 780-5
[19] Vanhegan et al.; Journal of Bone and Joint Surgery, 2012, vol. 94-B, No. 5, 619-23
[20] Weber et al.; Bio Med Research International, volume 2018, Article ID 8987104

1. A DNA-probe according to formula (I) or (II) comprising
a) a DNA-binder (DNAB);
b) a tracer (T)
c) optionally a linker (L) connecting the DNA-binder (DNAB) and the tracer (T) 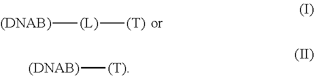
or a chelate complex with a metal ion, optionally a radioactive metal ion thereof.

2. The DNA-probe according to claim 1, wherein the DNA-binder (DNAB) is selected from the group consisting of 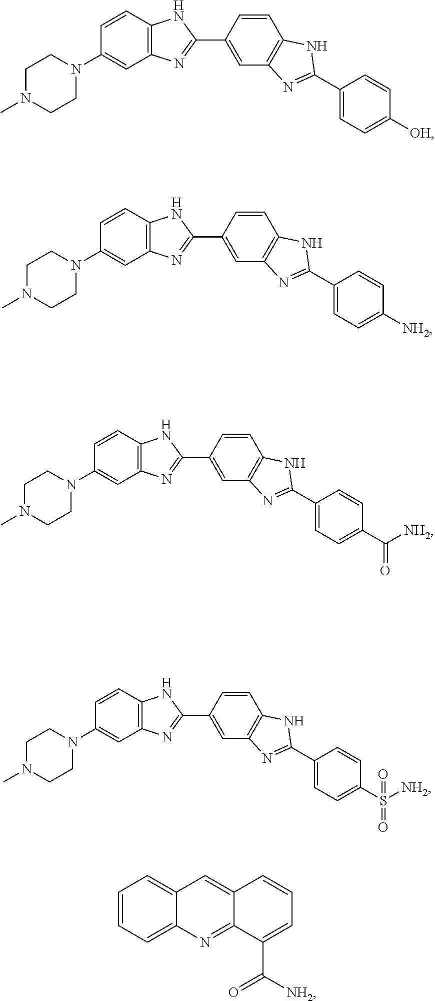 [SMILES: CN1CCN(c2ccc3[nH]c(-c4ccc5[nH]c(-c6ccc(O)cc6)nc5c4)nc3c2)CC1 ; CN1CCN(c2ccc3[nH]c(-c4ccc5[nH]c(-c6ccc(N)cc6)nc5c4)nc3c2)CC1 ; CN1CCN(c2ccc3[nH]c(-c4ccc5[nH]c(-c6ccc(C(N)=O)cc6)nc5c4)nc3c2)CC1 ; CN1CCN(c2ccc3[nH]c(-c4ccc5[nH]c(-c6ccc(S(N)(=O)=O)cc6)nc5c4)nc3c2)CC1 ; NC(=O)c1cccc2cc3ccccc3nc12] 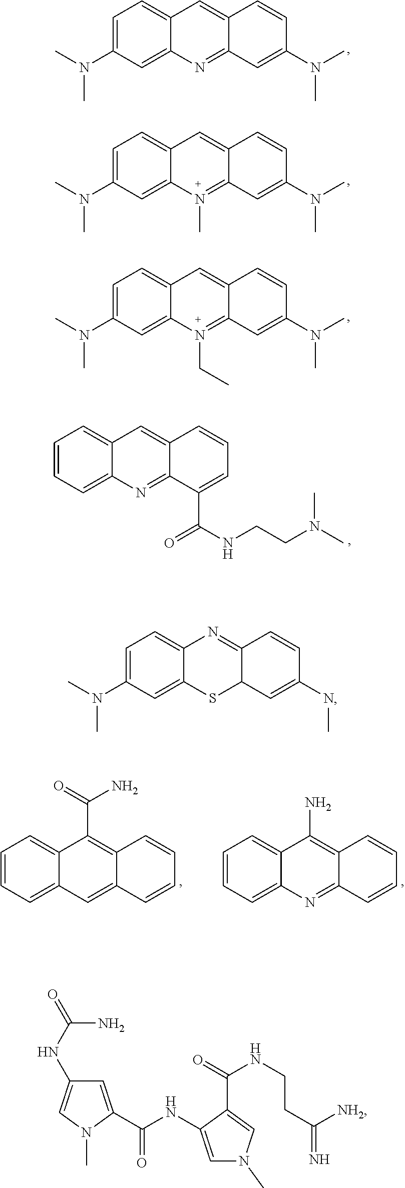 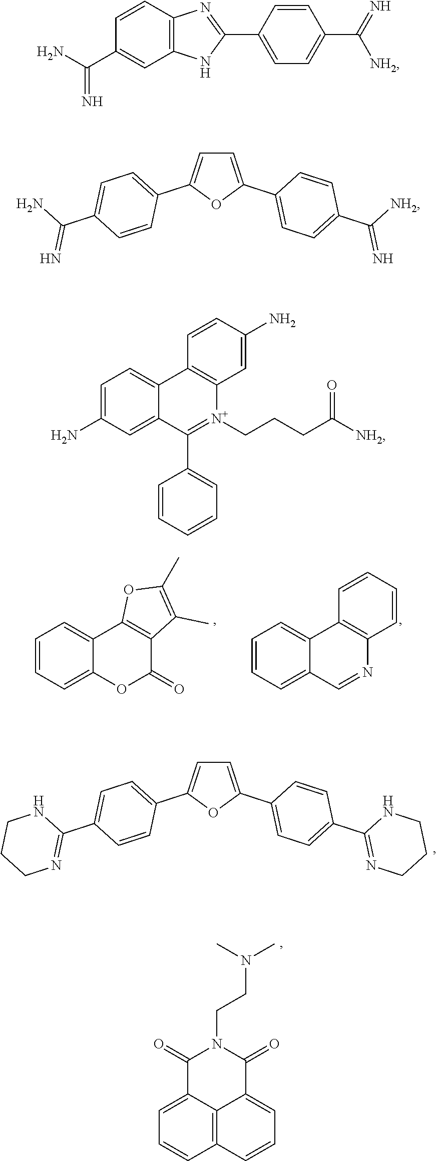 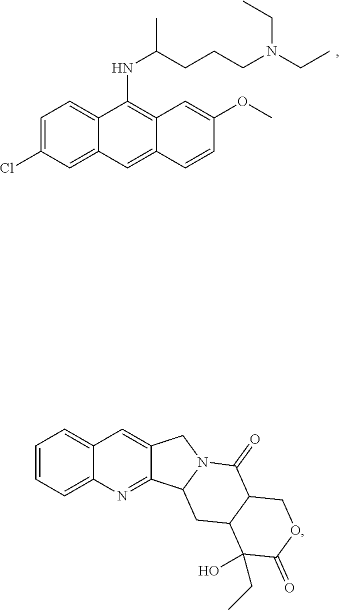 [SMILES: CCN(CC)CCCC(C)Nc1c2ccc(Cl)cc2cc2ccc(OC)cc12 ; CCC1(O)C(=O)OCC2C(=O)N3Cc4cc5ccccc5nc4C3CC21]
wherein
a) a hydrogen atom at a heteroatom is replaced by Y, wherein Y is connected to the linker (L) or to the tracer (T) or
b) a hydrogen atom connected to a carbon atom is replaced by Z, wherein Z is connected to the linker (L) or to the tracer (T);
and wherein
Y is a bond;
Z is selected from a group consisting of —O—,—S—, —NH—, —N((C₁-C₅)alkyl)- or a bond.

3. The DNA-probe according to claim 1, wherein the tracer (T) is selected from the group consisting of

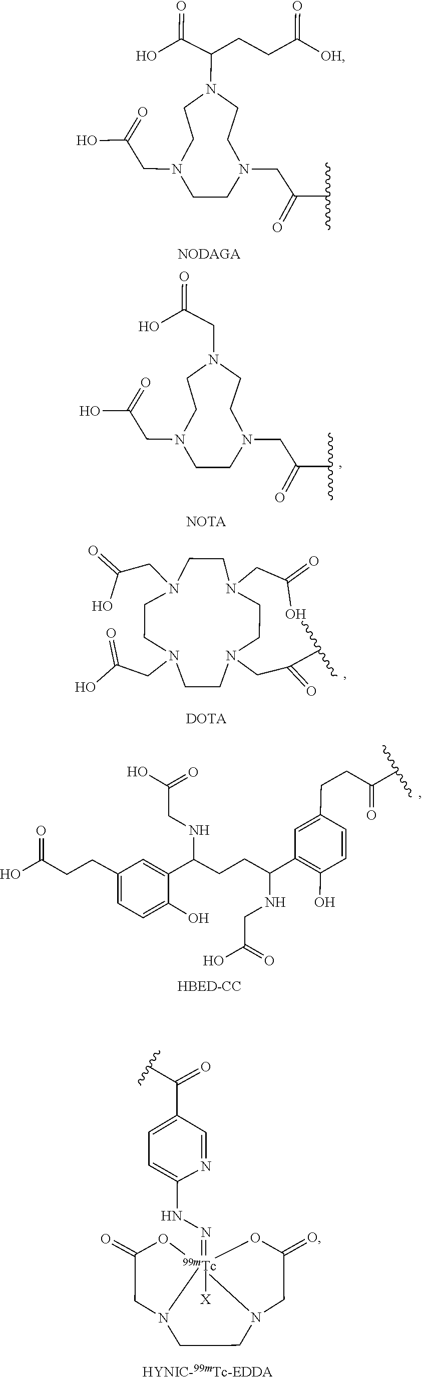

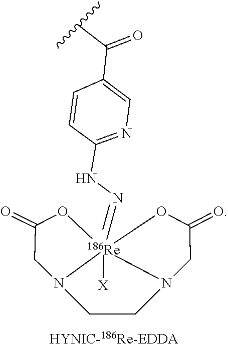

4. The DNA-probe according to claim 1, wherein the tracer (T) is

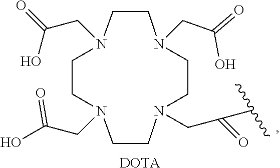

5. The DNA-probe according to claim 3, wherein
i) the tracer (T) forms a chelate complex with a radioactive metal, preferably optionally selected from the group consisting of ¹⁸⁶Re, ^{99m}Tc, ¹¹¹In, ¹⁷⁷Lu, ⁸⁹Zr, ⁶⁴Cu, ⁶⁸Ga, ⁴⁴Sc, ⁶⁰Co, ¹⁵³Gd or ii)
iia) NODAGA forms a chelate complex with a radioactive metal, optionally selected from the group consisting of ¹¹¹In, ¹⁷⁷Lu, ⁶⁴Cu, ⁶⁸Ga, ⁴⁴Sc and/or
iib) NOTA forms a chelate complex with a radioactive metal, optionally selected from the group consisting of ¹¹¹In, ¹⁷⁷Lu, ⁶⁴Cu, ⁶⁸Ga, ⁴⁴Sc and/or
iic) DOTA forms a chelate complex with a radioactive metal, optionally selected from the group consisting of ¹⁸⁶Re, ^{99m}Tc, ¹¹¹In, ¹⁷⁷Lu, ⁸⁹Zr, ⁶⁴Cu, ⁶⁸Ga, ⁶⁰Co, ⁴⁴Sc and ¹⁵³Gd.

6. The DNA-probe according to any one of claim 1, wherein the overall Linker (L) comprises the following parts connected as follows: 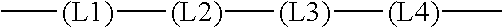
wherein the (L1) end is connected to (DNAB) via Y or Z;
wherein (L2), if present, is optionally connected with more than one (L3) and/or more than one (L1);
each of (L3), if present, is connected to (L4);
at least one (L4) end is connected to the tracer (T); optionally, one or more further (L4) may be present which are not connected with the tracer (T); each respective (L4) is at most connected to one tracer (T);
(L1) is selected from the group consisting of, 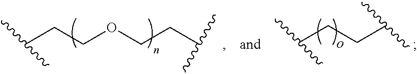
n is an integer between 1 and 10; optionally between 1 and 6, between 1 and 3, or 3;
o is an integer between 1 and 10; optionally between 1 and 6, between 1 and 3, or 3;
(L2) is selected from the group consisting of absent, 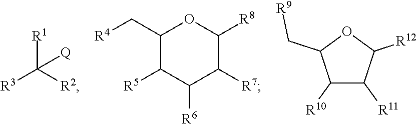
wherein Q is selected from the group consisting of H, —NH—, —NH₂
R¹, R², R³, R⁴, R⁵, R⁶, R⁷, R⁸, R⁹, R¹⁰, R¹¹, and R¹² are independently selected from the group consisting of —(CH₂)ₚOH, —(CH₂)ₚO—, —(CH₂)ₚNH₂, —(CH₂)ₚNH—, —(CH₂)ₚSH, —(CH₂)ₚS—, —(CH₂)ₚPhOH, —(CH₂)ₚPhO—, —NH₂, —NHC(O)— or N(C₁-C₆)alkylC(O)—, —NH—, —COOH, —(CH₂)ₚSO₃H, —CONH—, —(CH2)ₚOPO₃—, —(CH₂)ₚOSO₂—, —(CH₂)ₚOSO₂O—, —(CH₂)ₚOSO₂OH, —(CH₂)ₚOSO₂NH₂, —(CH₂)ₚOSO₂NH—, 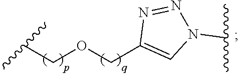
wherein in (L2) at least two of Q, R¹, R², R³ R⁴, R⁵, R⁶, R⁷, R⁸, R⁹, R¹⁰, R¹¹, or R¹² are selected from the group consisting of —(CH₂)ₚO—, —(CH₂)ₚNH—, —(CH₂)ₚS—, —(CH₂)ₚPhO—, —NHC(O)— or N(C₁-C₆)alkylC(O)—, —NH—, —CONH—, —(CH₂)ₚOPO₃—, —(CH₂)ₚOSO₂—, —(CH₂)ₚOSO₂O—, —(CH₂)ₚOSO₂NH—, 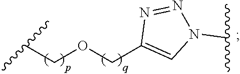
p is an integer between 0 and 10; optionally 1 and 3, or 1;
q is an integer between 1 and 4, optionally 1 and 3, or 1;
wherein, if any one of Q, R¹, R², R³ R⁴, R⁵, R⁶, R⁷, R⁸, R⁹, R¹⁰, R¹¹, or R¹² is independently selected from the group consisting of —(CH₂)ₚOH, —(CH₂)ₚNH₂, —(CH₂)ₚSH, —(CH₂)ₚPhOH, —NH₂, —COOH, —(CH₂)ₚSO₃H, —(CH₂)ₚOSO₂OH, —(CH₂)ₚOSO₂NH₂, then for the respective Q, R¹, R², R³ R⁴, R⁵, R⁶, R⁷, R⁸, R⁹, R¹⁰, R¹¹ or R¹², selected from this group (L3) and (L4) are absent;
(L3) is selected from the group consisting of absent, 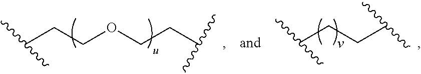
u is an integer between 1 and 10; optionally 1 and 6, bet-ween 1 and 3, or 3;
v is an integer between 1 and 10; optionally between 1 and 6, between 1 and 3, or 3;
(L4) is independently selected from the group consisting of absent, —NH—, —(CH₂)ₛSO₃H, —(CH₂)ₛOH, —(CH₂)ₛNH₂, —(CH₂)ₛPhOH, —NH₂, —COOH, —(CH₂)ₛOSO₂OH, —(CH₂)ₛOSO₂NH₂, —(CH₂)ₛOSO₂NH—, optionally —NH—;
If (L4) is independently selected from the group consisting of —NH—, and —(CH₂)ₛOSO₂NH— then (L4) is connected to a tracer (T);
s is an integer between 0 and 10; optionally 1 and 3, or 1.

7. The DNA-probe according to claim 1, wherein in the DNA-probe
a) the DNA-binder (DNAB) is selected from the group consisting of 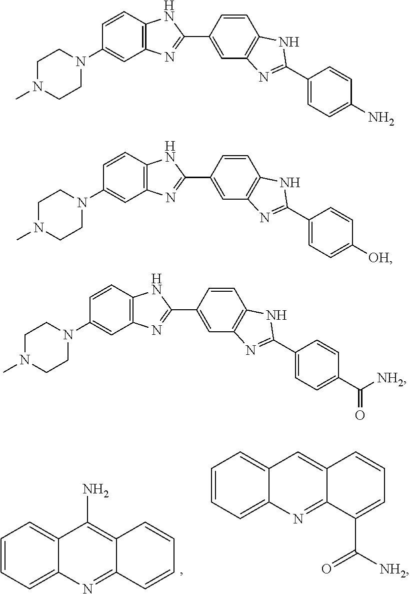 [SMILES: CN1CCN(c2ccc3[nH]c(-c4ccc5[nH]c(-c6ccc(N)cc6)nc5c4)nc3c2)CC1 ; CN1CCN(c2ccc3[nH]c(-c4ccc5[nH]c(-c6ccc(O)cc6)nc5c4)nc3c2)CC1 ; CN1CCN(c2ccc3[nH]c(-c4ccc5[nH]c(-c6ccc(C(N)=O)cc6)nc5c4)nc3c2)CC1 ; Nc1c2ccccc2nc2ccccc12 ; NC(=O)c1cccc2cc3ccccc3nc12]
and/or
b) the tracer (T) is selected from the group consisting of 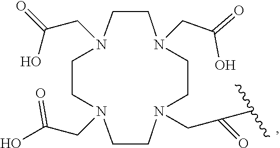 [SMILES: *C(=O)CN1CCN(CC(=O)O)CCN(CC(=O)O)CCN(CC(=O)O)CC1]
and/or
c) in the linker (L),
i) (L1) is 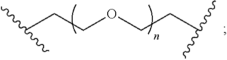
and/or
n is an integer between 1 and 10; optionally between 1 and 6, between 1 and 3, or 3;
ii) (L2) is absent, or 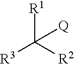
wherein Q is selected from the group consisting of H—NH—, —NH₂
R¹, R², or R³ are independently selected from the group consisting of —(CH₂)ₚOH, —(CH₂)ₚO—, —(CH₂)ₚNH₂, —(CH₂)ₚNH—, —(CH₂)ₚSH, —(CH₂)ₚS—, —(CH₂)ₛSO₃H, —(CH₂)ₚPhOH, —(CH₂)ₚPhO—, —NH₂, —NHC(O)— or N(C₁-C₆)alkylC(O)—, —NH—, —COOH, —CONH—, —(CH₂)ₚOPO₃—, —(CH₂)ₚOSO₂—, —(CH₂)ₚOSO₂O—, —(CH₂)ₚOSO₂OH, —(CH₂)ₚOSO₂NH₂, —(CH₂)ₚOSO₂NH—, 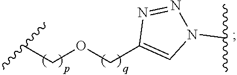
wherein in (L2) at least two of Q, R¹, R², or R³ are selected from the group consisting of —(CH₂)ₚO—, —(CH₂)ₚNH—, —(CH₂)ₚS—, —(CH₂)ₚPhO—, —NHC(O)— or N(C₁-C₆)alkylC(O)—, —NH—, —CONH—, (CH₂)ₚOPO₃—, (CH₂)ₚOSO₂—, (CH₂)ₚOSO₂O—, (CH₂)ₚOSO₂NH—, 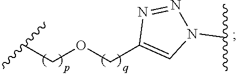
p is an integer between 0 and 10; optionally 1 and 3, or 1;
q is an integer between 1 and 4, optionally preferably-1 and 3, or nee-preferably 1; and/or
iii) (L3) is absent, or 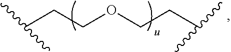
u is an integer between 1 and 10; optionally between 1 and 6, between 1 and 3, or 3;
iv) (L4) is —NH—.

8. The DNA-probe according to claim 1, wherein the DNA-probe is selected from the group consisting of, 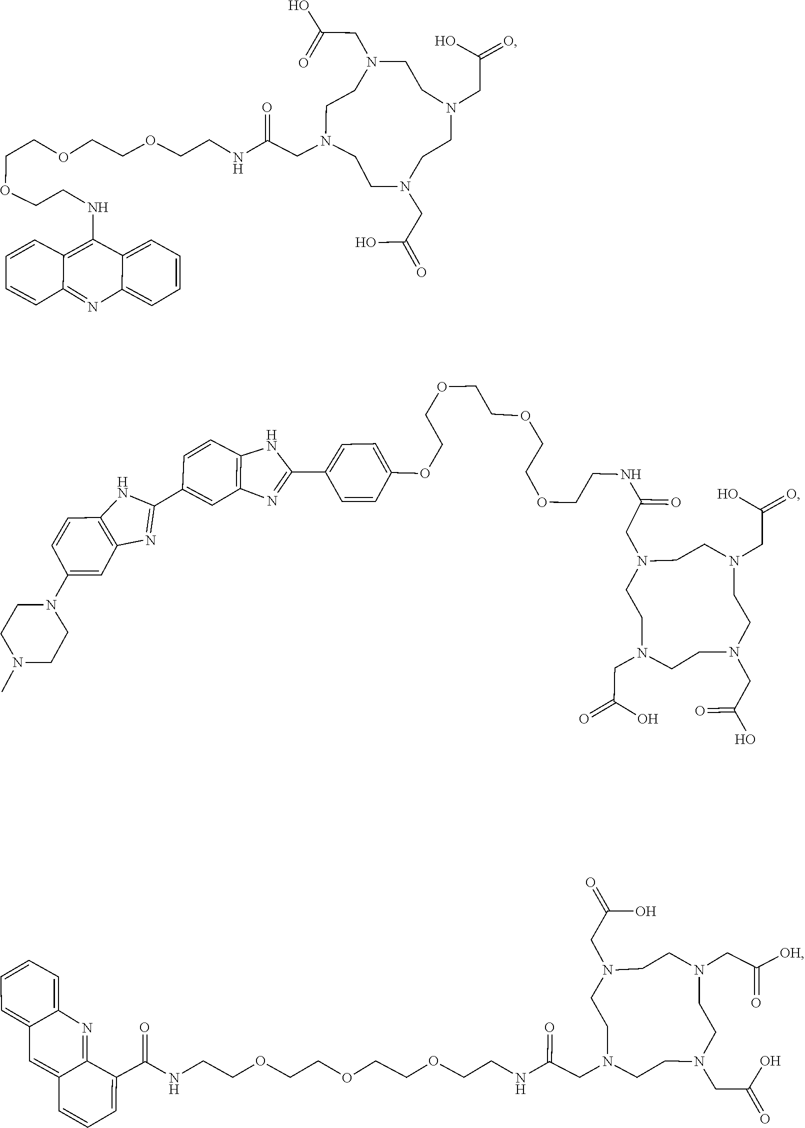 [SMILES: O=C(O)CN1CCN(CC(=O)O)CCN(CC(=O)NCCOCCOCCOCCNc2c3ccccc3nc3ccccc23)CCN(CC(=O)O)CC1 ; CN1CCN(c2ccc3[nH]c(-c4ccc5[nH]c(-c6ccc(OCCOCCOCCOCCNC(=O)CN7CCN(CC(=O)O)CCN(CC(=O)O)CCN(CC(=O)O)CC7)cc6)nc5c4)nc3c2)CC1 ; O=C(O)CN1CCN(CC(=O)O)CCN(CC(=O)NCCOCCOCCOCCNC(=O)c2cccc3cc4ccccc4nc23)CCN(CC(=O)O)CC1] 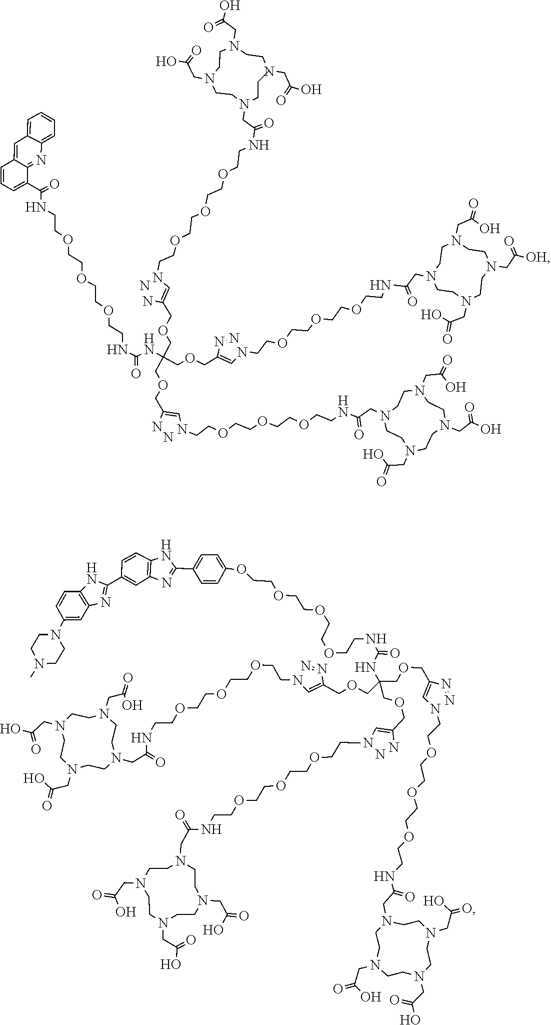 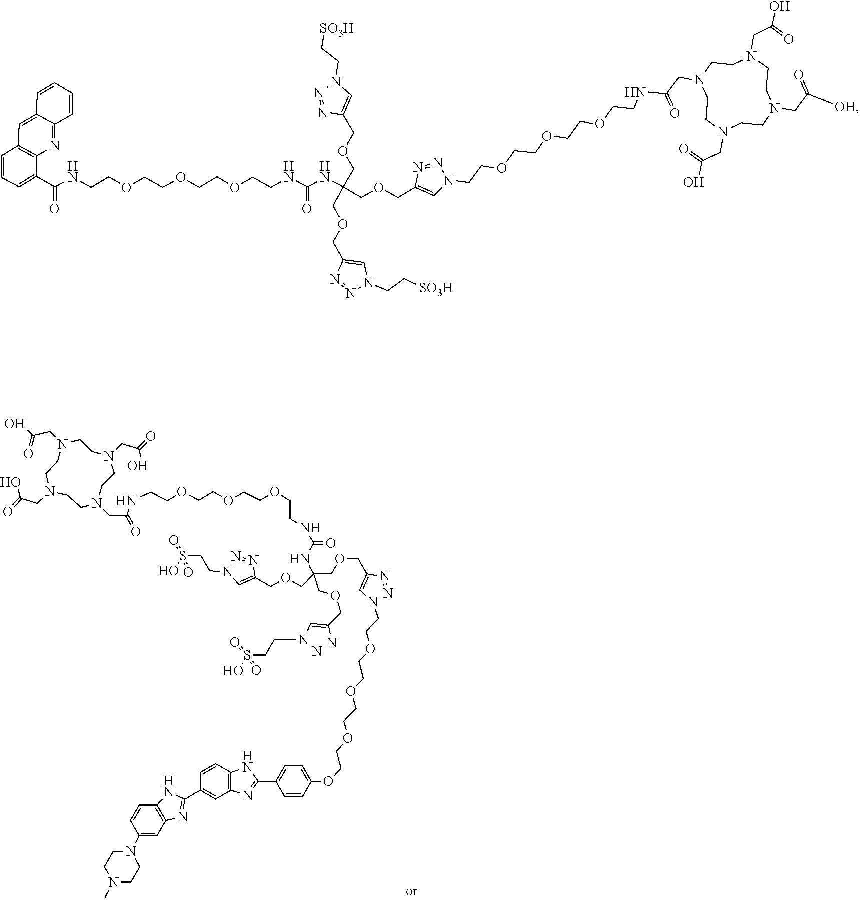 [SMILES: O=C(O)CN1CCN(CC(=O)O)CCN(CC(=O)NCCOCCOCCOCCn2cc(COCC(COCc3cn(CCS(=O)(=O)O)nn3)(COCc3cn(CCS(=O)(=O)O)nn3)NC(=O)NCCOCCOCCOCCNC(=O)c3cccc4cc5ccccc5nc34)nn2)CCN(CC(=O)O)CC1 ; CN1CCN(c2ccc3[nH]c(-c4ccc5[nH]c(-c6ccc(OCCOCCOCCOCCn7cc(COCC(COCc8cn(CCS(=O)(=O)O)nn8)(COCc8cn(CCS(=O)(=O)O)nn8)NC(=O)NCCOCCOCCOCCNC(=O)CN8CCN(CC(=O)O)CCN(CC(=O)O)CCN(CC(=O)O)CC8)nn7)cc6)nc5c4)nc3c2)CC1] 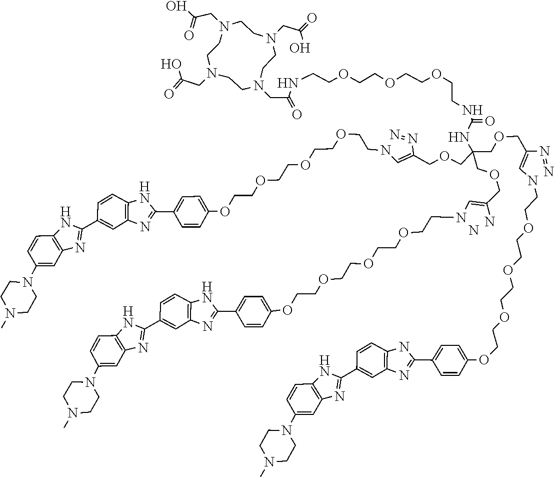
or a chelate complex with a metal ion, optionally a radioactive metal ion thereof or a metal selected from the group consisting of ¹⁸⁶Re, ^{99m}Tc, ¹¹¹In, ¹⁷⁷Lu, ⁸⁹Zr, ⁶⁴Cu, ⁶⁸Ga, ⁴⁴Sc, ⁶⁰Co, or ¹⁵³Gd thereof.

9. A method for the detection of extracellular DNA of bacteria comprising detecting extracellular DNA of bacteria by applying the DNA-probe of claim 1.
